(19) United States
(12) Patent Application Publication
(10) Pub. No.: US 2024/0277829 A1
(43) Pub. Date: Aug. 22, 2024

(54) HIV-1 VACCINATION AND SAMT-247 MICROBICIDE TO PREVENT HIV-1 INFECTION

(71) Applicant: THE U.S.A., as represented by the Secretary, Department of Health and Human Services, Bethesda, MD (US)

(72) Inventors: Genoveffa Franchini, Bethesda, MD (US); Marjorie Robert-Guroff, Rockville, MD (US); Daniel Howard Appella, Bethesda, MD (US); Sabrina Helmold Hait, Bethesda, MD (US); Mohammad Arif Rahman, Bethesda, MD (US); Massimiliano Bissa, Bethesda, MD (US); Ettore Appella, Bethesda, MD (US); Lisa M. Miller Jenkins, Bethesda, MD (US); Isabela Silva de Castro, Bethesda, MD (US); James D. Stamos, Bethesda, MD (US)

(73) Assignee: THE U.S.A., as represented by the Secretary, Department of Health and Human Services, Bethesda, MD (US)

(21) Appl. No.: 18/681,019

(22) PCT Filed: Aug. 2, 2022
(86) PCT No.: PCT/US2022/074432

§ 371 Date: Feb. 2, 2024

Related U.S. Application Data
(60) Provisional application No. 63/228,707, filed on Aug. 3, 2021.

(51) Int. Cl. A61K 39/21; A61K 31/255; A61K 39/00; A61P 31/18; A61P 37/04

(52) U.S. Cl. CPC A61K 39/21; A61K 31/255; A61P 31/18; A61P 37/04; A61K 2039/54; A61K 2039/55505; C12N 2740/16122; C12N 2740/16134; C12N 2740/16171; C12N 2740/16222; C12N 2740/16234; C12N 2740/16271

(57) ABSTRACT

Methods are disclosed for inhibiting a HIV infection in a subject. These methods include administering to the subject an effective amount of a recombinant gp120 protein comprising a deletion of HIV-1 Envelope residues 137-152 according to the HXBc2 numbering system, or a nucleic acid molecule encoding the recombinant gp120 protein, wherein the recombinant gp120 protein elicits an immune response to HIV-1. The methods also include administering to the subject an effective amount of a SAMT-247 microbicide.

### CROSS REFERENCE TO RELATED APPLICATIONS

This claims the benefit of U.S. Provisional Application No. 63/228,707, filed Aug. 3, 2021, which is incorporated herein by reference.

### STATEMENT OF GOVERNMENT SUPPORT

This invention was made with Government support under project number Z01 #: ZIA BC 011126 and ZIA BC 011063 by the National Institutes of Health, National Cancer Institute, and under project number Z01 #: DK075135 by the National Institutes of Health, National Institute of Diabetes and Digestive and Kidney Diseases. The United States Government has certain rights in the invention.

### FIELD OF THE DISCLOSURE

This disclosure relates to a method of inhibiting a human immunodeficiency virus (HIV) infection in a subject that includes the administration of an effective amount of a recombinant gp120 protein that includes V1 domain deletion and an effective amount of a SAMT-247 microbicide.

### BACKGROUND

Data from UNAIDS in 2020 indicated that almost 75 million people have become infected with HIV since the start of the HIV pandemic, and 32 million people have died from AIDS-related illnesses. Currently, the highest burden of people living with HIV as well as new HIV infections and deaths from AIDS is in Africa. About 61% of the 5500 new weekly infections worldwide occur in sub-Saharan Africa, mostly in adolescent girls (age 15-19) who have no access to antiretroviral therapy for treatment or prevention. Resource-rich countries have access to Pre-Exposure Prophylaxis (PrEP), an antiretroviral treatment that prevents HIV infection. Adherence to daily, life-long treatment is crippled by stigmatism and limits compliance. Thus, a vaccine and other treatment options to halt HIV infection remain priorities, particularly for resource-deprived and underserved populations. The rising infection rates among vulnerable populations worldwide highlight the need to develop an effective method for inhibiting HIV infections.

### SUMMARY OF THE DISCLOSURE

Methods are disclosed for inhibiting a HIV infection in a subject. These methods include administering to the subject an effective amount of a recombinant gp120 protein comprising a deletion of HIV-1 Envelope (Env) residues 137-152 according to the HXBc2 numbering system, or a nucleic acid molecule encoding the recombinant gp120 protein, wherein the recombinant gp120 protein elicits an immune response to HIV-1. The methods also include administering to the subject an effective amount of a SAMT-247 microbicide. In some embodiments, the recombination gp120 protein is administered to the subject in a composition that further comprises an adjuvant. In other non-limiting examples, the methods include a prime boost immunization. In further non-limiting examples, the effective amount of SAMT-247 is administered intravaginally.

Methods are also disclosed for inhibiting HIV-1 acquisition in a subject, that include administering to the subject an effective amount of a composition comprising a prime immunization of a DNA vector encoding HIV-1 Env with a deletion of HIV-1 Env residues 137-152 according to the HXBc2 numbering system and an adjuvant, administering to the subject a boost immunization of a vector encoding HIV env, HIV gag, and HIV pol and an alum adjuvant, and administering to the subject a boost immunization of a purified gp120 protein with a deletion of HIV-1 Env residues 137-152 according to the HXBc2 numbering system formulated with an effective amount of an alum adjuvant; and applying intra-vaginally an effective amount of a SAMT-247 microbicide, thereby inhibiting the HIV-1 acquisition the subject.

The foregoing and other features and advantages of the invention will become more apparent from the following detailed description of several embodiments which proceeds with reference to the accompanying figures.

### BRIEF DESCRIPTION OF THE FIGURES

FIGS. 1A-1G: Schematic representation of the immunization regimen, infection rate, and SIV VL. A) Fifty rhesus macaques were subdivided into four groups: vaccine (n=18), vaccine+SAMT-247 (n=20), SAMT-247 (n=6), and controls (n=6). Thirty-eight animals were primed with DNA-SIVgp160 ΔV1⁺ SIV_{mac239} gag and boosted with ALVAC-SIV encoding env, gag, and pol and ALVAC-SIV+ΔV1 gp120 protein in alum hydroxide at the indicated timepoints. Twelve animals remained naïve until SIV challenge. Beginning at week 17, protective efficacy against SIV_{mac251} was assessed by subjecting all animals to up to 14 weekly intravaginal viral exposures (arrows) in the presence or absence of SAMT-247 until infection was confirmed. Animals either received 0.8% SAMT-247 in HEC gel (n=26) or HEC gel only (n=24) 4 h prior to each low-dose SIV_{mac251} challenge. B) Significant protection in the vaccine group compared with concurrent+historical controls. C) Significant protection in the vaccine+SAMT-247 group compared with concurrent+historical controls. D) Delayed SIV acquisition in the vaccine microbicide group was seen compared with the vaccine only group. E) No differences in delayed acquisition in the SAMT-247 group compared to the concurrent plus historical controls. F) Comparison of peak VL at week 2 among different groups of animals. G) VL geometric means of all macaque groups over time. Data shown in (B-E) were analyzed with log-rank (Mantel-Cox) test. Data shown in (F) was analyzed with Mann Whitney U test. Horizontal and vertical bars denote mean and SD respectively (*p<0.05 and **p<0.01). Circles indicate vaccine group, squares indicate vaccine+SAMT-247 group, triangle indicates SAMT-247 group, and the control group is shown.

FIGS. 2A-2G. Ex vivo and in vitro quantification of humoral and NK responses. A) Correlation of V2-specific (NCI05) ADCC killing with number of intra-vaginal challenges in the vaccine group (n=18). B) Comparison of SAMT-247 non-treated/treated effector cell mediated ADCC killing in vaccine and vaccine+SAMT-247 group (n=38). In all of the figures that following, circles are the vaccine only group and squares are the vaccine+SAMT-247 group. C) Correlation of SAMT-247-induced ADCC killing with number of intra-vaginal challenges in the vaccine+SAMT-247 group (n=20). D, E) Intracellular Granzyme B, perforin, IFN-γ, and TNF-α in macaque rectal mucosal NKG2A⁺ cells in the presence or absence of different stimuli (n=9). F) Correlation of rectal mucosal Env-specific NKp44⁺IL-17⁺ cells with number of intra-vaginal challenges in the vaccine group (n=18). G) Macaque rectal mucosal NKp44⁺IL-17⁺ cells in the presence or absence of different stimuli (n=9). Data shown in (A, C, F) were analyzed with Spearman correlation test. Data shown in (B, D, E, G) were analyzed with Wilcoxon signed rank test. Horizontal and vertical bars denote mean and SD (*p<0.05, **p<0.01, ****p<0.0001). Circles indicate vaccine group, squares indicate vaccine+SAMT-247 group, and triangles indicates unstimulated, PMA stimulated, and PMA+SAMT-247 stimulated rectal cells from aged matched animals, as shown.

FIGS. 3A-3E. Evaluation of CD14⁺ cell mediated efferocytosis responses. A) Correlation of normalized efferocytosis with number of intra-vaginal challenges in the vaccine group animals (n=18). B) Correlation of SAMT-247 induced normalized efferocytosis (SAMT-247 not treated normalized efferocytosis subtracted from SAMT-247 treated normalized efferocytosis) with number of intra-vaginal challenges in the vaccine+SAMT-247 group animals (n=20). C) Frequency of CD14⁺ classical monocytes upon gp120 pooled peptide and gp120 pooled peptide+SAMT-247 stimulation in vaccine group (n=4) and vaccine+SAMT-247 group (n=6). D, E) Percentage of efferocytosis and efferocytosis MFI by CD14⁺ monocytes in the presence/absence of different stimuli. Data shown in (A, B) were analyzed with Spearman correlation test. Data shown in (C-E) were analyzed with Wilcoxon signed rank test. Horizontal and vertical bars denote mean and SD (*p<0.05, **p<0.01). Circles indicate vaccine group and squares indicate vaccine+SAMT-247 group.

FIGS. 4A-4H: Evaluation of ex vivo and in vitro T cell responses. A, B) Correlation of CCR5⁺α₄β₇⁺ and CCR5⁻α₄β₇⁻ memory Th1 cells with number of intra-vaginal challenges in the vaccine+SAMT-247 group animals (n=20). C, D) Evaluation of CCR5 and α₄β₇ markers on Th1 and Th2 cells in the absence or presence of stimuli in the vaccine+SAMT-247 group animals (n=9). E, F) Correlation of gp120 peptide+SAMT-247 stimulated CCR5⁻α₄β₇⁻ Th1 and Th2 cells with number of intra-vaginal challenges in the vaccine+SAMT-247 group animals (n=9). G, H) IFN-γ⁺, TNF-α⁺ and IL-10⁺ Th1 and Th2 cells in the rectal mucosa in the absence or presence of stimuli (n=9). Data shown in (A, B, E, F) were analyzed with the Spearman correlation test. Data shown in (C, D, G, H) were analyzed with the Wilcoxon signed rank test. Horizontal and vertical bars denote mean and SD (*p<0.05, **p<0.01). A square indicates vaccine+SAMT-247 group, Triangles indicate unstimulated, PMA stimulated, and PMA+SAMT-247 stimulated rectal cells from aged matched animals, as shown.

FIGS. 5A-5B. Understanding Zinc intensity in human NK cells. A) Representative imaging of human NK cells unstimulated/stimulated with SAMT-247, PMA, or PMA+SAMT-247. Immunofluorescence staining for Zinc and nuclei. B) Mean Zinc intensity of the healthy human donor in the presence/absence of zinc chelator in different conditions of stimulation. Fluorescence intensity of each field was measured for green color and total number of DAPI positive cells were counted to determine the mean intensity of zinc/cells using iMARIS software. Eight separate donors were used, and duplicate field was evaluated for the calculation. Wilcoxon signed rank test and Mann Whitney unpaired test were performed. Horizontal and vertical bars denote mean and SD. *p<0.05, **p<0.01. Diamonds indicate unstimulated, SAMT-247 stimulated, PMA stimulated, and PMA+SAMT-247 stimulated NK cells, as indicated, from healthy human donors, respectively.

FIGS. 6A-6J. Evaluation of cytokine responses upon SAMT-247 stimulation. A, B) Radar plots comparing different expressions of granzyme B, perforin and cytokines by NKG2A⁺ cells from vaccinated animals at week 17 in the absence or presence of zinc chelator (n=6). C) Radar plots comparing CD14 marker expression on monocytes, and D) Intracellular IL-10 expression by CD14⁺ monocytes in the absence or presence of zinc chelator (n=6). E-J) Radar plots comparing different expressions of cytokines by different subsets of Th1 and Th2 cells from vaccinated animals at week 17 in the absence or presence of zinc chelator (n=6). Data shown in (A=J) were analyzed with Wilcoxon signed rank test. The radar plot represents the mean percentage value of cytokine responses. Straight line represents without Zinc chelator and dotted line with zinc chelator. *p<0.05.

FIGS. 7A-7H. Quantification of humoral responses in plasma of rhesus macaques in response to immunization. A) Plasma antibody titers against ΔV1 gp120 over the course of immunization. B) Pepscan of plasma against different V1 and V2 loop peptides. Comparison of C) ADCC titer, D) V2- specific (NCI05 specific) ADCC killing and E) V2-specific (NCI09 specific) ADCC killing at week 17 in the vaccine+SAMT-247 group (n=20) and vaccine group (n=18). Correlation of F) ADCC killing, G) ADCC titer and H) V2-specific (NCI09 specific) ADCC killing with number of intra-vaginal challenges in the vaccine group animals (n=18). Data shown in (A, C-E) were analyzed with Wilcoxon signed rank test. F-H were analyzed with Spearman correlation test. Horizontal and vertical bars denote mean and SD. ***p<0.001, ****p<0.0001. Circles indicate vaccine group, and squares indicate vaccine+SAMT-247 group.

FIGS. 8A-8G Ex vivo evaluation of NK/ILCs. A, B) Intracellular Granzyme B, perforin, IFN-γ and TNF-α in healthy human blood NKG2A⁺ cells in the presence or absence of different stimuli (n=6). C) Comparison of Env-specific rectal NKp44⁺IL-17⁺ cells between vaccine+SAMT-247 (n=20) and vaccine group (n=18). D) Gating of NKG2A⁺NK cells, NKp44⁺ILCs and NKG2A⁻ NKp44⁻ILCs in the rectal mucosal sample in the presence of PMA or PMA+SAMT-247 at 12 hours post stimulation. E) Gating of NKp44⁺IL-17⁺ ILCs in the rectal mucosal sample in the presence of PMA or PMA+SAMT-247 at 12 hours post stimulation. F) Frequency of NKG2A⁺NK cells, NKp44⁺ILCs and NKG2A⁻NKp44⁻ILCs in the rectal mucosal sample in the presence or absence of stimuli (n=9). G) Macaque rectal mucosal NKG2A⁻NKp44⁻IFN-γ cells in the presence or absence of different stimuli (n=9). Data shown in (A, B and E-G) were analyzed with Wilcoxon signed rank test. Horizontal and vertical bars denote mean and SD. *p<0.05, **p<0.01. Circles indicate vaccine group, and squares indicate vaccine+SAMT-247 group. Diamonds indicate unstimulated, SAMT-247 stimulated, PMA stimulated, and PMA+SAMT-247 stimulated PBMCs from healthy human donors, respectively. Triangles indicate unstimulated, PMA stimulated, and PMA+SAMT-247 stimulated rectal cells from aged matched animals, as shown.

FIGS. 9A-9D. Ex vivo assessment efferocytosis. Comparison of A, C) percentage of efferocytosis and B, D) Efferocytosis MFI using pre and week 14 CD14⁺ monocytes in all vaccinated animals (n=38). Data shown in (A-D) were analyzed with Wilcoxon signed rank test. Horizontal and vertical bars denote mean and SD. ****p<0.0001. Circles indicate vaccine group, and squares indicate vaccine+SAMT-247 group.

FIGS. 10A-10G. Evaluation of T cell responses. A) Gating strategy of Th1 and Th2 cells, B, C) Comparison of CCR5⁺α₄β₇⁺ and D, E) CCR5⁻α₄β₇⁻ memory Th1 and Th2 cells pre and 1 week post last vaccination (week 13) in blood (n=38). F, G) Frequency of Th1 cells and Th2 cells in the rectal mucosa of macaques (n=9). Data shown in (B-G) were analyzed with Wilcoxon signed rank test. Horizontal and vertical bars denote mean and SD. *p<0.05, ****p<0.0001. Circles indicate vaccine group, and squares indicate vaccine+SAMT-247 group. Triangles indicate unstimulated, PMA stimulated, and PMA+SAMT-247 stimulated rectal cells from aged-matched animals, as shown.

FIGS. 11A-11D. Quantification of macaque blood NK cell responses in the presence of stimulation. A-D) Comparison of expressions of granzyme B, perforin, IFN-γ and TNF-α by macaque blood NKG2A⁺ cells from week 17 in the absence or presence of zinc chelator and stimuli (n=9). Data shown in (A-D) were analyzed with Wilcoxon signed rank test. Horizontal and vertical bars denote mean and SD. *p<0.05. Here, Circles indicate vaccine group, and squares indicate vaccine+SAMT-247 group.

FIGS. 12A-12F: Analysis of monkey and human NK cell responses in the presence of stimulation. A) Comparison of expressions of NKG2A marker in the absence or presence of zinc chelator and stimuli (n=6). B-E) Comparison of expressions of granzyme B, perforin, IFN-γ and TNF-α by NKG2A⁺ cells from healthy humans in the absence or presence of zinc chelator and stimuli (n=6). F) Radar plots comparing different expressions of granzyme B, perforin and cytokines by NKG2A⁺ cells from human blood in the absence or presence of zinc chelator and stimuli (n=6). Data shown in (A-F) were analyzed with Wilcoxon signed rank test. The radar plot represents the mean percentage value of cytokine responses. Straight line represents without Zinc chelator and dotted line with zinc chelator. Horizontal and vertical bars denote mean and SD. *p<0.05. Circles indicate vaccine group, and squares indicate vaccine+SAMT-247 group. Diamonds indicate unstimulated, SAMT-247 stimulated, PMA stimulated, and PMA+SAMT-247 stimulated PBMCs from healthy human donors, respectively.

FIGS. 134A-13B Assessment of the CD14⁺ monocyte and CD14⁺IL-10⁺ monocyte responses in the presence of stimulation. A) Comparison of expressions of CD14 marker and B) CD14⁺IL-10⁺ monocytes in the absence or presence of zinc chelator and stimuli (n=6). Data shown in (A=B) were analyzed with Wilcoxon signed rank test. Horizontal and vertical bars denote mean and SD. *p<0.05. Circles indicate vaccine group, and squares indicate vaccine+SAMT-247 group.

FIGS. 14A-14D. Evaluation of CCR5^{+/-} and α₄β₇^{+/-} T cell responses in the presence of stimulation. A-D) Comparison of expressions of CCR5 and α₄β₇ markers in Th1 and Th2 memory cells in the absence or presence of zinc chelator and stimuli (n=6). Data shown in (A-D) were analyzed with Wilcoxon signed rank test. Horizontal and vertical bars denote mean and SD. *p<0.05. Circles indicate vaccine group, and squares indicate vaccine+SAMT-247 group.

FIGS. 15A-15F Comparison of cytokine responses by CCR5⁺α₄β₇⁺ and CCR5⁻α₄β₇⁻ memory Th1 cells in the presence of stimulation. A-F) Comparison of intracellular expressions of IFN-γ, TNF-α and IL-10 by CCR5⁺α₄β₇⁺ and CCR5⁻α₄β₇⁻ Th1 memory cells in the absence or presence of zinc chelator and stimuli (n=6). A-F were analyzed with Wilcoxon signed rank test. Horizontal and vertical bars denote mean and SD. *p<0.05. Circles indicate vaccine group, and squares indicate vaccine+SAMT-247 group.

FIGS. 16A-16F Evaluation of cytokine responses by CCR5⁺α₄β₇⁺ and CCR5⁻α₄β₇⁻ memory Th2 cells in the presence of stimulation. A-F) Comparison of intracellular IFN-γ, TNF-α, and IL-10 cytokines by CCR5⁺α₄β₇⁺ and CCR5⁻α₄β₇⁻ Th2 memory cells in the absence or presence of zinc chelator and stimuli (n=6). Data shown in (A-F) were analyzed with Wilcoxon signed rank test. Horizontal and vertical bars denote mean and SD. *p<0.05. Circles indicate vaccine group, and squares indicate vaccine+SAMT-247 group.

FIG. 17. Evaluation of in vitro SAMT-247 release rate from different intra-vaginal rings. In vitro SAMT-247 release from pod-intravaginal rings (IVRs) (mean±standard deviation (SD)) is linear and scales with exposed surface area. Inset shows zoomed view of low releasing profiles: # represents 3×1.5 mm diameter delivery channels (1.15 mg d⁻¹); @ represents 1×1.5 mm diameter delivery channels (0.63 mg d⁻¹); * represents 1×1.0 mm diameter delivery channels (0.063 mg d⁻¹); \hat{ } represents 1×0.75 mm diameter delivery channels (0.039 mg d⁻¹). FIG. 18. Schematic representation of the immunization regimen. Fifty rhesus macaques are subdivided into four groups: vaccine+SAMT-247 IVRs (n=15), SAMT-247 IVRs (n=15), empty IVRs (n=10) and controls (n=10). Fifteen animals are primed with DNA-SIVgp160 ΔV1⁺ SIV_{mac239} gag and boosted with ALVAC-SIV encoding env, gag, and pol and ALVAC-SIV+ΔV1 gp120 protein in alum hydroxide at the indicated timepoints. Thirty-five animals remain naïve until SIV challenge. At week 11, 16, 20, 24 and 28 intravaginal rings (IVRs) with or without SAMT-247 are inserted. Beginning at week 17, protective efficacy against SIV_{mac251} is assessed by subjecting all animals to up to 14 weekly intravaginal viral exposures (arrows).

### SEQUENCE LISTING

The nucleic and amino acid sequences listed herein are shown using standard letter abbreviations for nucleotide bases, and one letter code for amino acids, as defined in 37 C.F.R. 1.822. Only one strand of each nucleic acid sequence is shown, but the complementary strand is understood as included by any reference to the displayed strand. The Sequence Listing is submitted as an ST.26 file [Sequence_Listing, Aug. 2, 2022, 25.7 KB], which is incorporated by reference herein.

SEQ ID NO: 1 is the amino acid sequence of HIV-1 Env of HXB2.

SEQ ID NOs: 2-10 are amino acid sequences of a recombinant gp120 protein.

SEQ ID NO: 11 is the amino acid sequence of a cleavage site.

SEQ ID NO: 12 is the amino acid sequence of a portion of a modified cleavage site.

SEQ ID NO: 13 is the amino acid sequence of a linker.

SEQ ID NO: 14 is the amino acid sequence of a BG505 TM domain.

SEQ ID NOs: 15 and 16 are the amino acid sequences of an influenza A domain.

SEQ ID NO: 17 is the amino acid sequence of a foldon domain.

SEQ ID NO: 18 is a nucleic acid sequence encoding a V1 deleted HIV-1 Env sequence.

### DETAILED DESCRIPTION OF SEVERAL EMBODIMENTS

The development of fully effective interventions to halt HIV transmission requires an understanding of the ability of cellular and humoral immunities to inhibit HIV infection and tissue seeding. A Canarypox-based HIV vaccine regimen, with a bivalent gp120 boost in alum, so far has been the only vaccine to demonstrate modest efficacy in both macaques (SIVmac251 model) and in humans (RV144 trial). The use of MF59 as adjuvant to the Canarypox-based vaccines instead of alum was found to be ineffective in both macaques and humans (HVTN702), supporting the relevance of the SIVmac251 macaque model. The efficacy of ALVAC-based vaccines has been improved by shortening the vaccine regimen from 6 to 3 months, by substituting the ALVAC-SIV prime with a V1-deleted envelope DNA immunogen, and by boosting with a monovalent V1-deleted gp120 protein in alum. This regimen decreases the risk of SIVmac251 acquisition by approximately 70% in female macaques following exposure to the neutralization-resistant SIVmac251 that mirrors circulating HIV-1.

The macaque model was instrumental for identifying novel innate immunity correlates of risk. Functional and phenotypic analyses of immune cell populations, analyses of gene expression (via RNA-seq) and chromatin marks (ATAC-seq), revealed that the efficacy of these vaccine modalities is linked to the induction of anti-inflammatory monocyte innate immunity together with V2-specific antibody-dependent cytotoxicity and tolerant T cell responses. Vaccine-induced epigenetic chromatin marks in monocytes linked to the activation of cyclic-AMP pathway and the engagement of T-reg. These data are consistent with the suppression of type I interferon expression in monocytes and increased expression of IL-10 and PD1 in T-cells and the induction of efferocytosis. Strikingly, following false discovery rate analyses of all RNA-seq gene expression data derived from the blood of animals before and after vaccination, a single gene, encoding the ZC3H7A zinc finger RNA binding protein, was identified whose vaccine-induced downregulation appeared to be necessary for vaccine efficacy. To further increase protection from infection, a combination of a V1-deleted vaccine was combined with the use of an effective amount of SAMT-247, a Zinc finger protein inhibitor. The combined use of the recombinant gp120 protein comprising a deletion of HIV-1 Env residues 137-152 according to the HXBc2 numbering system and a SAMT-247 microbicide provided a synergistic effect.

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particularly suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Antibody-Dependent Cellular Cytotoxicity: A mechanism of cell-mediated immune defense whereby an effector cell of the immune system actively lyses a target cell, whose membrane-surface antigens have been bound by specific antibodies. ADCC is one of the mechanisms through which antibodies can act to limit and contain infection. ADCC is independent of the immune complement system that also lyses targets but does not require any other cell. ADCC requires an effector cell, such as natural killer (NK) cells that typically interact with immunoglobulin G (IgG) antibodies. Macrophages, neutrophils, and eosinophils can also mediate ADCC.

Adjuvant: A component of an immunogenic composition used to enhance antigenicity. In some embodiments, an adjuvant can include a suspension of minerals (alum, aluminum hydroxide, or phosphate) on which antigen is adsorbed; or water-in-oil emulsion, for example, in which antigen solution is emulsified in mineral oil (Freund's incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in an immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants of use include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill in the art is familiar with adjuvants (see, e.g., Singh (ed.) Vaccine Adjuvants and Delivery Systems. Wiley-Interscience, 2007). Adjuvants can be used with an effective amount of a recombinant gp120 protein comprising a deletion of HIV-1 Envelope (Env) residues 137-152 according to the HXBc2 numbering system, or a nucleic acid molecule encoding the recombinant gp120 protein.

Administration: The introduction of a composition into a subject by a chosen route. Administration can be local or systemic. For example, if the chosen route is intravenous, the composition (such as a composition including an immunogen) is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid. In some examples, an amino acid in a polypeptide is substituted with an amino acid from a homologous polypeptide, for example, an amino acid in a recombinant Clade A HIV-1 Env polypeptide can be substituted with the corresponding amino acid from a Clade B HIV-1 Env polypeptide.

Antibody: An immunoglobulin, antigen-binding fragment, or derivative thereof, that specifically binds and recognizes an analyte (antigen), such as HIV-1 Env. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010). Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The framework region of an antibody, that is the combined framework regions of the constituent light and heavy chains, serves to position and align the CDRs in three-dimensional space. The CDRs are primarily responsible for binding to an epitope of an antigen.

Biological sample: A sample of biological material obtained from a subject. Biological samples include all clinical samples useful for detection of disease or infection (e.g., HIV infection) in subjects. Appropriate samples include any conventional biological samples, including clinical samples obtained from a human or veterinary subject. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, cerebrospinal fluid (CSF), etc.), tissue biopsies or autopsies, fine-needle aspirates, and/or tissue sections. In a particular example, a biological sample is obtained from a subject having, suspected of having or at risk of having HIV infection.

Carrier: An immunogenic molecule to which an antigen (such as gp120) can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Conservative variants: “Conservative” amino acid substitutions are those substitutions that do not substantially affect or decrease a function of a protein, such as the ability of the protein to elicit an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that affect or decrease an activity or function of the recombinant Env protein, such as the ability to elicit an immune response when administered to a subject. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form. Contacting includes contact between one molecule and another molecule, for example the amino acid on the surface of one polypeptide, such as an antigen, that contacts another polypeptide, such as an antibody. Contacting also includes administration, such as administration of a disclosed antigen to a subject by a chosen route.

Control: A reference standard. In some embodiments, the control is a negative control sample obtained from a healthy patient. In other embodiments, the control is a positive control sample obtained from a patient diagnosed with HIV-1 infection. In still other embodiments, the control is a historical control or standard reference value or range of values (such as a previously tested control sample, such as a group of HIV-1 patients with known prognosis or outcome, or group of samples that represent baseline or normal values).

A difference between a test sample and a control can be an increase or conversely a decrease. The difference can be a qualitative difference or a quantitative difference, for example, a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detecting: To identify the existence, presence, or fact of something. General methods of detecting are known to the skilled artisan and may be supplemented with the protocols and reagents disclosed herein. Detection can include a physical readout, such as fluorescence or a reaction output, or the results of a PCR assay.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

Effective amount: An amount of agent, such as an HIV immunogen or SAMT-247, that is sufficient to elicit a desired response, such as an anti-viral response in a subject. With regard to an immunogen, is understood that to obtain a protective immune response against an antigen of interest can require multiple administrations, and/or administration as the “prime” in a prime boost protocol wherein the boost immunogen can be different from the prime immunogen. Accordingly, an effective amount of an immunogen can be the amount of the immunogen sufficient to elicit a priming immune response in a subject that can be subsequently boosted with the same or a different immunogen to elicit a protective immune response. With regard to SAMT-247, administration of an effective amount of a SAMT-247 microbicide can include administering SAMT-247 itself, or a prodrug of SAMT-247 that is metabolized to the active form in a subject.

In one example, a desired response is to inhibit, reduce or prevent HIV-1 infection. The HIV-1 infection does not need to be completely eliminated or reduced or prevented for the method to be effective. For example, administration can decrease the HIV-1 infection (for example, as measured by infection of cells, or by number or percentage of subjects infected by HIV-1) by a desired amount, for example by at least 50%, at least 60%, at least 70%, at least 80%, at least 90%, at least 95%, at least 98%, or even at least 100% (elimination or prevention of detectable HIV-1 infection), as compared to a suitable control.

Efferocytosis: The process by which apoptotic cells are removed by phagocytic cells. During efferocytosis, the cell membrane of phagocytic cells engulfs an apoptotic cell, forming a large fluid-filled vesicle containing the dead cell. This ingested vesicle is called an efferosome. The process is similar to macropinocytosis. The effect of efferocytosis is that apoptotic cells are removed before their membrane integrity is breached and their contents leak into the surrounding tissue. See Henson, P. M., Annu Rev Cell Dev Biol 33, 127-144 (2017).

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression control sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat;

the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

Heterologous: A heterologous polypeptide or polynucleotide refers to a polypeptide or polynucleotide derived from a different source or species.

Human Immunodeficiency Virus Type 1 (HIV-1): A retrovirus that causes immunosuppression in humans (HIV-1 disease) and leads to a disease complex known as the acquired immunodeficiency syndrome (AIDS). “HIV-1 disease” refers to a well-recognized constellation of signs and symptoms (including the development of opportunistic infections) in persons who are infected by an HIV-1 virus, as determined by antibody or western blot studies. Laboratory findings associated with this disease include a progressive decline in T cells. Related viruses that are used as animal models include simian immunodeficiency virus (SIV), and feline immunodeficiency virus (FIV). Treatment of HIV-1 with HAART has been effective in reducing the viral burden and ameliorating the effects of HIV-1 infection in infected individuals.

HIV-1 broadly neutralizing antibody: An antibody that reduces the infectious titer of HIV-1 by binding to HIV-1 Envelope protein and inhibiting HIV-1 function. In some embodiments, broadly neutralizing antibodies to HIV are distinct from other antibodies to HIV in that they neutralize a high percentage (such as at least 50% or at least 80%) of the many types of HIV in circulation. Non-limiting examples of HIV-1 broadly neutralizing antibodies include PG9, VRC01, and N6.

HIV-1 envelope protein (Env): The HIV-1 Env protein is initially synthesized as a precursor protein of 845-870 amino acids in size. Individual precursor polypeptides form a homotrimer and undergo glycosylation within the Golgi apparatus as well as processing to remove the signal peptide, and cleavage by a cellular protease between approximately positions 511/512 to generate separate gp120 and gp41 polypeptide chains, which remain associated as gp120-gp41 protomers within the homotrimer. The ectodomain (that is, the extracellular portion) of the HIV-1 Env trimer undergoes several structural rearrangements from a prefusion closed conformation that evades antibody recognition, through intermediate conformations that bind to receptors CD4 and co-receptor (either CCR5 or CXCR4), to a postfusion conformation. The HIV-1 Env ectodomain comprises the gp120 protein (approximately HIV-1 Env positions 31-511) and the gp41 ectodomain (approximately HIV-1 Env positions 512-664). An HIV-1 Env ectodomain trimer comprises a protein complex of three HIV-1 Env ectodomains. As used herein “HIV-1 Env ectodomain trimer” includes both soluble trimers (that is, trimers without gp41 transmembrane domain or cytoplasmic tail) and membrane anchored trimers (for example, trimers including a full-length gp41).

Mature gp120 includes approximately HIV-1 Env residues 31-511, contains most of the external, surface-exposed, domains of the HIV-1 Env trimer, and it is gp120 which binds both to cellular CD4 receptors and to cellular chemokine receptors (such as CCR5). The mature gp120 wild-type polypeptide is heavily N-glycosylated, giving rise to an apparent molecular weight of 120 kD. Native gp120 includes five conserved regions (C1-C5) and five regions of high variability (V1-V5).

Variable region 1 and Variable Region 2 (V1/V2 domain) of gp120 include ˜50-90 residues which contain two of the most variable portions of HIV-1 (the V1 loop and the V2 loop), and one in ten residues of the V1/V2 domain are N-glycosylated. Despite the diversity and glycosylation of the V1/V2 domain, a number of broadly neutralizing human antibodies have been identified that target this region, including the somatically related antibodies PG9 and PG16 (Walker et al., Science, 326:285-289, 2009). In certain examples the V1/V2 domain includes gp120 position 126-196.

Mature gp41 includes approximately HIV-1 Env residues 512-860, and includes cytosolic-, transmembrane-, and ecto-domains. The gp41 ectodomain (including approximately HIV-1 Env residues 512-644) can interact with gp120 to form an HIV-1 Env protomer that trimerizes to form the HIV-1 Env trimer.

A standardized numbering scheme for HIV-1 Env proteins (the HXBc2 numbering system) is set forth in Numbering Positions in HIV Relative to HXB2CG Bette Korber et al., Human Retroviruses and AIDS 1998: A Compilation and Analysis of Nucleic Acid and Amino Acid Sequences. Korber et al., Eds. Theoretical Biology and Biophysics Group, Los Alamos National Laboratory, Los Alamos, NM, which is incorporated by reference herein in its entirety. For reference, the amino acid sequence of HIV-1 Env of HXB2 is set forth as SEQ ID NO: 1 (GENBANK® GI:1906382, incorporated by reference herein).

HXBc2 (Clade B, SEQ ID NO: 1):
MRVKEKYQHLWRWGWRWGTMLLGMLMICSATEKLWVTVYYGVPVWKEAT
TTLFCASDAKAYDTEVHNVWATHACVPTDPNPQEVVLVNVTENFNMWKN
DMVEQMHEDIISLWDQSLKPCVKLTPLCVSLKCTDLKNDININSSSGRM
IMEKGEIKNCSFNISTSIRGKVQKEYAFFYKLDIIPIDNDTTSYKLISC
NTSVITQACPKVSFEPIPIHYCAPAGFAILKCNNKTFNGTGPCTNVSTV
QCTHGIRPVVSTQLLINGSLAEEEVVIRSVNFTDNAKTIIVQLNTSVEI
NCTRPNNNTRKRIRIQRGPGRAFVTIGKIGNMRQAHCNISRAKWNNTLK
QIASKLREQFGNNKTIIFKQSSGGDPEIVTHSFNCGGEFFYCNSTQLFN
STWFNSTWSTEGSNNTEGSDTITLPCRIKQIINMWQKVGKAMYAPPISG
QIRCSSNITGLLLTRDGGNSNNESEIFRPGGGDMRDNWRSELYKYKVVK
IEPLGVAPTKAKRRVVQREKRAVGIGALFLGFLGAAGSTMGAASMTLTV
QARQLLSGIVQQQNNLLRAIEAQQHLLQLTVWGIKQLQARILAVERYLK
DQQLLGIWGCSGKLICTTAVPWNASWSNKSLEQIWNHTTWMEWDREINN
YTSLIHSLIEESQNQQEKNEQELLELDKWASLWNWFNITNWLWYIKLFI
MIVGGLVGLRIVFAVLSIVNRVRQGYSPLSFQTHLPTPRGPDRPEGIEE
EGGERDRDRSIRLVNGSLALIWDDLRSLCLFSYHRLRDLLLIVTRIVEL
LGRRGWEALKYWWNLLQYWSQELKNSAVSLLNATAIAVAEGTDRVIEVV
QGACRAIRHIPRRIRQGLERILL

HIV-1 gp140: A recombinant HIV Env polypeptide including gp120 and the gp41 ectodomain, but not the gp41 transmembrane or cytosolic domains. HIV-1 gp140 polypeptides can trimerize to form a soluble HIV-1 Env ectodomain trimer.

HIV-1 gp145: A recombinant HIV Env polypeptide including gp120, the gp41 ectodomain, and the gp41 transmembrane domain. HIV-1 gp145 polypeptides can trimerize to form a membrane-anchored HIV-1 Env ectodomain trimers.

HIV-1 gp160: A recombinant HIV Env polypeptide including gp120 and the entire gp41 protein (ectodomain, transmembrane domain, and cytosolic tail).

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immunogenic conjugate: A composition composed of at least two heterologous molecules (such as an HIV-1 Env trimer and a carrier, such as a protein carrier) linked together that stimulates or elicits an immune response to a molecule in the conjugate in a vertebrate. In some embodiments where the conjugate includes a viral antigen, the immune response is protective in that it enables the vertebrate animal to better resist infection from the virus from which the antigen is derived.

Immune response: A response of a cell of the immune system, such as a B cell, T cell, or monocyte, to a stimulus, such as a vaccination or an infection. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies. “Priming an immune response” refers to treatment of a subject with a “prime” immunogen to induce an immune response that is subsequently “boosted” with a boost immunogen. Together, the prime and boost immunizations produce the desired immune response in the subject. “Enhancing an immune response” refers to co-administration of an adjuvant and an immunogenic agent, wherein the adjuvant increases the desired immune response to the immunogenic agent compared to administration of the immunogenic agent to the subject in the absence of the adjuvant.

Immunogen: A protein or a portion thereof that is capable of inducing an immune response in a mammal, such as a mammal infected or at risk of infection with a pathogen.

Immunogenic composition: A composition comprising a disclosed immunogen, or a nucleic acid molecule or vector encoding a disclosed immunogen, that elicits a measurable CTL response against the immunogen, or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). For in vivo use, the immunogenic composition will typically include the protein or nucleic acid molecule in a pharmaceutically acceptable carrier and may also include other agents, such as an adjuvant.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Linked: The term “linked” means joined together, either directly or indirectly. For example, a first moiety may be covalently or noncovalently (e.g., electrostatically) linked to a second moiety. This includes, but is not limited to, covalently bonding one molecule to another molecule, noncovalently bonding one molecule to another (e.g. electrostatically bonding), non-covalently bonding one molecule to another molecule by hydrogen bonding, non-covalently bonding one molecule to another molecule by van der Waals forces, and any and all combinations of such couplings. Indirect attachment is possible, such as by using a “linker”. In several embodiments, linked components are associated in a chemical or physical manner so that the components are not freely dispersible from one another, at least until contacting a cell, such as an immune cell.

Linker: One or more molecules or groups of atoms positioned between two moieties. Typically, linkers are bifunctional, i.e., the linker includes a functional group at each end, wherein the functional groups are used to couple the linker to the two moieties. The two functional groups may be the same, i.e., a homobifunctional linker, or different, i.e., a heterobifunctional linker. In several embodiments, a peptide linker can be used to link the C-terminus of a first protein to the N-terminus of a second protein. Non-limiting examples of peptide linkers include glycine-serine peptide linkers, which are typically not more than 10 amino acids in length. Typically, such linkage is accomplished using molecular biology techniques to genetically manipulate DNA encoding the first polypeptide linked to the second polypeptide by the peptide linker.

Native protein, sequence, or disulfide bond: A polypeptide, sequence or disulfide bond that has not been modified, for example, by selective mutation. For example, selective mutation to focus the antigenicity of the antigen to a target epitope, or to introduce a disulfide bond into a protein that does not occur in the native protein. Native protein or native sequence are also referred to as wild-type protein or wild-type sequence. A non-native disulfide bond is a disulfide bond that is not present in a native protein, for example, a disulfide bond that forms in a protein due to introduction of one or more cysteine residues into the protein by genetic engineering.

Natural killer cell: A lymphoid cell that does not express clonally distributed receptors for antigen. In vivo, natural killer cells are primarily in the peripheral blood, spleen and bone marrow, but can migrate to inflamed tissues in response to chemoattractants. Natural killer cells play a role in the natural defense from viruses, pathogens and tumors. Upon activation, they release cytokines and chemokines that induce inflammatory processes, modulate hematopoiesis, and affect monocyte and granulocyte cell growth and function, see Moretta et al., Nature Immunology 3(1): 6-8, 2002.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxyribonucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular embodiments, suitable for administration to a subject the carrier may be sterile, and/or suspended or otherwise contained in a unit dosage form containing one or more measured doses of the composition suitable to elicit the desired anti-HIV-1 immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymers as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with protein, and is used herein to refer to a polymer of amino acid residues.

Prime-boost immunization: An immunotherapy including administration of multiple immunogens over a period of time to elicit the desired immune response.

Recombinant: A recombinant nucleic acid is one that has a sequence that is not naturally occurring or has a sequence that is made by an artificial combination of two otherwise separated segments of sequence. This artificial combination can be accomplished, for example, the artificial manipulation of isolated segments of nucleic acids, for example, using genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

RV144 Trial: A phase III clinical trial of a prime-boost HIV-1 vaccine that was carried out in Thailand. The immunization protocol consisted of four injections of ALVAC HIV (vCP1521) followed by two injections of AIDSVAX B/E. ALVAC HIV (vCP1521) is a canarypox vector genetically engineered to express HIV-1 Gag and Pro (subtype B LAI strain) and CRF01_AE (subtype E) HIV-1 gp120 (92TH023) linked to the transmembrane anchoring portion of gp41 (LAI). AIDSVAX B/E is a bivalent HIV gp120 envelope glycoprotein vaccine containing a subtype E envelope from the HIV-1 strain A244 (CM244) and a subtype B envelope from the HIV-1 MN each produced in Chinese hamster ovary cell lines. The envelope glycoproteins, 300 μg of each, were co-formulated with 600 μg of alum adjuvant. The RV144 trial, ALVAC HIV (vCP1521), and AIDSVAX B/E are described in Rerks-Ngarm et al. (New Eng J Med. 361 (23):2209-2220, 2009, incorporated by reference herein). In some embodiments, the Env ectodomain encoding portion of ALVAC HIV (vCP1521) and the gp120 proteins of AIDSVAX B/E can be modified to encode or contain the V1 deletion provided herein (deletion of residues 137-152 according to HXBc2 numbering) and administered to a subject using the rv144 prime-boost protocol (or any other suitable protocol).

SAMT-247: A compound of the formula C₁₂H₁₄N₂O₃S, and the chemical structure of:

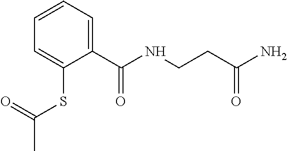 [SMILES: CC(=O)Sc1ccccc1C(=O)NCCC(N)=O]

SAMT-247 microbicides include SAMT-247 and pharmaceutically acceptable salts thereof. A prodrug form of SAMT-247 can provide an effective amount of SAMT-247, as it is metabolized in the subject to the active form.

Sensitivity and specificity: Statistical measurements of the performance of a binary classification test. Sensitivity measures the proportion of actual positives which are correctly identified (e.g., the percentage of samples that are identified as including nucleic acid from a particular virus). Specificity measures the proportion of negatives which are correctly identified (e.g., the percentage of samples that are identified as not including nucleic acid from a particular virus).

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 18-30 amino acids in length) that directs newly synthesized secretory or membrane proteins to and through membranes (for example, the endoplasmic reticulum membrane). Signal peptides are typically located at the N-terminus of a polypeptide and are removed by signal peptidases after the polypeptide has crossed the membrane. Signal peptide sequences typically contain three common structural features: an N-terminal polar basic region (n-region), a hydrophobic core, and a hydrophilic c-region). Exemplary signal peptide sequences are set forth as residues 1-11 of SEQ ID NOs: 10, 6, and 7.

Specifically bind: When referring to the formation of an antibody:antigen protein complex, or a protein:protein complex, refers to a binding reaction which determines the presence of a target protein, peptide, or polysaccharide (for example, a glycoprotein), in the presence of a heterogeneous population of proteins and other biologics. Thus, under designated conditions, a particular antibody or protein binds preferentially to a particular target protein, peptide or polysaccharide (such as an antigen present on the surface of a pathogen, for example, gp120) and does not bind in a significant amount to other proteins or polysaccharides present in the sample or subject. Specific binding can be determined by standard methods. A first protein or antibody specifically binds to a target protein when the interaction has a K_{D} of less than 10⁻⁷ Molar, such as less than 10⁻⁸ Molar, less than 10⁻⁹, or even less than 10¹⁰ Molar.

Subject: Living multicellular vertebrate organisms, a category that includes human and non-human mammals. In an example, a subject is a human. In an additional example, a subject is selected that is in need of inhibiting of an HIV-1 infection. For example, the subject is either uninfected and at risk of HIV-1 infection or is infected in need of treatment.

Transmembrane domain: An amino acid sequence that inserts into a lipid bilayer, such as the lipid bilayer of a cell or virus or virus-like particle. A transmembrane domain can be used to anchor an antigen to a membrane.

Treating or inhibiting HIV-1: Inhibiting the full development of HIV-1 in a subject who is at risk for or has an HIV-1 infection or acquired immunodeficiency syndrome (AIDS). “Treatment” refers to a therapeutic intervention that ameliorates a sign or symptom of HIV-1 infection in an infected subject. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Inhibiting HIV-1 in an uninfected subject refers to a reduction in infection rate or likelihood of infection. The term “reduces” or “inhibits” is a relative term, such that an agent reduces acquisition of an HIV infection, or if the HIV infection is quantitatively diminished following administration of the agent, as compared to a reference agent or other control. An immunogenic composition that induces an immune response that inhibits HIV-1, can, but does not necessarily completely, inhibit HIV-1 infection of a subject (or group of subjects), so long as the infection is measurably diminished, for example, by at least about 50%, such as by at least about 70%, or about 80%, or even by about 90% of (that is to 10% or less than) the infection or response in the absence of the agent, or in comparison to a reference agent. Inhibiting HIV-1 infection includes inhibiting HIV-1 acquisition by a seronegative subject.

Under conditions sufficient for: A phrase that is used to describe any environment that permits a desired activity. In one example the desired activity is formation of an immune complex.

Vaccine: A pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine reduces the severity of the symptoms associated with HIV-1 infection and/or decreases the viral load compared to a control. In another non-limiting example, a vaccine reduces HIV-1 infection compared to a control.

Vaginal Ring or Intra-vaginal Ring: A doughnut-shaped polymeric drug delivery device which is designed to be inserted into the vagina of a female human in order to provide controlled release of an active agent to the vagina over an extended period of time. A “matrix ring” or “matrix-type ring” refers to an intravaginal ring in which the effective amount of a SAMT-247 microbicide is distributed in the ring, such as wherein the SAMT-247 is homogenously distributed in the ring. Matrix rings are typically manufactured by injection molding or extrusion of the active compound-containing active mix, leading to the uniform distribution of the active compounds throughout the ring. A “reservoir ring” refers to an intravaginal ring that includes a reservoir (a full or partial-length core), which is completely surrounded by a sheath. In one embodiment, the effective amount of a SAMT-247 microbicide is present in the core of a reservoir ring, with a blank sheath. Release rates can be modified by changing the nature or thickness of a rate-controlling sheath.

Vector: An entity containing a DNA or RNA molecule bearing a promoter(s) that is operationally linked to the coding sequence of an immunogenic protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses.

A non-limiting example of a DNA-based expression vector is pCDNA3.1, which includes a mammalian expression enhancer and promoter (such as a CMV promoter). Non-limiting examples of viral vectors include adeno-associated virus (AAV) vectors as well as Poxvirus vector (e.g., Vaccinia, MVA, avian Pox, or Adenovirus).

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. The presence of VLPs following recombinant expression of viral proteins can be detected using conventional techniques, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60: 1445-1456; and Hagensee et al. (1994) J. Virol. 68:4503-4505; Vincente, J Invertebr Pathol., 2011; Schneider-Ohrum and Ross, Curr. Top. Microbiol. Immunol., 354: 53073, 2012).

### II. Recombinant HIV-1 gp120 Proteins with a V1 Domain Deletion

Variable region 1 and Variable Region 2 (V1/V2) of the gp120 component of the viral spike are believed to both harbor key epitopes that could be targeted by the host immune system to reduce the risk of viral acquisition and contribute greatly to the antigenic variation and conformational masking that facilitates evasion of host antibody responses, including but not limited to neutralizing antibody responses. Localized to a membrane-distal, apical “cap,” which holds the spike in a neutralization-resistant conformation, V1/V2 is not essential for host cell entry, but removal in its entirety renders the virus sensitive to antibody-mediated neutralization. The ˜50-90 residues that comprise V1/V2 contain two of the most sequence-variable portions of the virus, and one in ten residues of V1/V2 are N-glycosylated. Despite the diversity and glycosylation of V1/V2, a number of broadly neutralizing and non-neutralizing, cross-reactive human antibodies have been identified that target this region. As discussed in the examples, the majority of these antibodies share specificity for the V2 portion of the V1V2 domain. However, despite extensive effort, immunogens embodying intact V1V2 have proven ineffective at eliciting a V2-based immune response that is protective against HIV-1 infection.

Of use in the presently disclosed methods are recombinant HIV-1 gp120 proteins that include a V1 domain deletion that unmasks epitopes targeted by protective immune responses, and which are shown to elicit a surprisingly effective immune response for viral inhibition, see PCT Publication No. WO 2020/086483, which is incorporated herein by reference. The modification comprises deletion of HXBc2 residues 137-152 from the gp120 protein, which, as discussed in the examples, exposes V2 epitopes and is shown to produce a protective immune response in an animal model. Isolated V1V2 domain proteins that contain the V1 deletion, as well as HIV-1 Env trimers containing the recombinant gp120 protein with the V1 deletion were also of use in inducing a protective immune response. Surprisingly, combined use of a V1 deleted immunogen with an effective amount of SAMT-247 acted synergistically to protect from an infection with HIV.

### A. Recombinant gp120 and HIV-1 Env Proteins Containing Same

Isolated immunogens are of use in the disclosed methods that include a recombinant gp120 protein that is modified to include a deletion of V1 residues 137-152 according to the HXBc2 numbering system. Deletion of these V1 residues exposes V2 epitopes on the gp120 protein, and immunogens including this modification elicit a protective immune response that targets the V2 epitopes.

HIV-1 can be classified into four groups: the “major” group M, the “outlier” group O, group N, and group P. Within group M, there are several genetically distinct clades (or subtypes) of HIV-1. Recombinant HIV-1 Env proteins can be derived from any type of HIV, such as groups M, N, O, or P, or clade, such as clade A, B, C, D, F, G, H, J, or K, and the like. HIV-1 Env proteins from the different HIV-1 clades, as well as nucleic acid sequences encoding such proteins and methods for the manipulation and insertion of such nucleic acid sequences into vectors, are known (see, e.g., HIV Sequence Compendium, Division of AIDS, National Institute of Allergy and Infectious Diseases (2013); HIV Sequence Database (hiv-web.lanl.gov/content/hiv-db/mainpage.html); see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013). Exemplary native HIV-1 Env protein sequences are available in the HIV Sequence Database (hiv-web.lanl.gov/content/hiv-db/mainpage.html).

In some embodiments, any of the recombinant gp120 immunogens can include the corresponding amino acid sequence from a native HIV-1 Env protein, for example, from genetic subtype A-F as available in the HIV Sequence Database (hiv-web.lanl.gov/content/hiv-db/mainpage.html), or an amino acid sequence at least 90% (such as at least 95%, at least 96%, at least 97%, at least 98% or at least 99%) identical thereto that has been modified to include a deletion of HXBc2 residues 137-152.

In some embodiments, the recombinant gp120 protein comprises or consists essentially of the amino acid sequence set forth as any one of:

SEQ ID NO: 2. gp120 of HIV-1 Env circulating
recombinant form AE, strain A244 (GenBank:
KU562843.1, incorporated by reference herein
in its entirety) with V1 137-152 deletion.
Deletion boundaries are underlined.
NLWKWGTLILGLVIICSASDNLWVTVYYGVPVWRDADTTLFCASDAKAQ
ETEAHNVWATHACVPTDPNPQELHLENVTENFNMWKNNMVEQMQEDVIS
LWDQSLKPCVKLTPLCVTLNCTNANLEVRNCSFNMTTELRDKKQKVHAL
FYKLDIVPIEDNTSSSKYRLINCNTSVIKQACPKISFDPIPIHYCTPAG
YAILKCNDKNFNGTGPCKNVSSVQCTHGIKPAVSTQLLLNGSLAEEEII
IRSENLTNNAKTIIVHLNKSVEINCTRPSNNTRTSINIGPGQVFYRTGD
IIGDIRKAYCEINGAKWNEVLKKVTEKLHEHFNNKTIIFQPPSGGDLEI
RMHHFNCRGEFFYCNTTRLFNNTCMENETMEGCNGTIIKPCKIKQIINM
WQRAGQAMYAPPISGRINCVSNITGILLTRDGGLNNTNETFRPGGGNIK
DNWRSELYKYKVVQIEPLGIAPTRAKRRVVEREKR
SEQ ID NO: 3. gp120 of HIV-1 Env clade B
strain MN (GenBank: AAL66251.1, incorporated by
reference herein in its entirety) with V1
137-152 deletion. Deletion boundaries are
underlined.
HWWGWGTMLLGLLMTICSATEKLWVTVYYGVPVWKEATTTLFCASDAKA
YDTEVHNVWATHACVPTDPNPQEVQLVNVTEDFNMWKNNMVEQMHEDII
SLWDQSLKPCVKLTPLCVTLNCTDLRNEMKNCSFNITTSIRDKMQKEYA
LLYKLDIVAIDKDNTSYRLISCNTSVITQACPKVSFEPIPIHYCAPAGF
AILKCNDKNFTGKGPCKNVSTVQCTHGIRPVVSTQLLLNGSLAEEEVVI
RSENFTNNAKTIIVHLNESVQINCTRPYNNRRTRIHIGPGRAFYTTKNI
KGTIRQAHCTISSAKWNDTLRQIVSKLKEQFKNKTIVFKQSSGGDPEIV
MHSFNCGGEFFYCNTSSLFNSTWNGNNTWNNTTGSNSNITLQCKIKQII
NMWQEVGKAMYAPPIEGQIRCSSNITGLLLTRDGGNDTDTNNTEIFRPG
GGDMRDNWRSELYKYKVVTIEPLGVAPTKAKRRVVQREKR
SEQ ID NO: 4. gp120 of HIV-1 Env clade C strain
96ZM651 (GenBank: AAK30970.1, incorporated by
reference herein in its entirety) with V1
137-152 deletion. Deletion boundaries are
underlined.
RWWTWGILGFWMLMICNVWGNLWVTVYYGVPVWKEAKTTLFCASDAKSY
EKEVHNVWATHACVPTDPNPQEIVLGNVTENFNMWKNDMDVQMHEDIIS
LWDQSLKPCVKLTPLCVTLNCTEVNVDMKNCSFNITTELKDKKKNVYAL
FYKLDIVSLNETDDSETGNSSKYYRLINCNTSALTQACPKVSFDPIPIH
YCAPAGYAILKCNNKTFNGTGPCHNVSTVQCTHGIKPVVSTQLLLNGSL
AEEGIIIRSENLTNNVKTIIVHLNRSIEIVCVRPNNNTRQSIRIGPGQT
FYATGDIIGDIRQAHCNISRTNWTKTLREVRNKLREHFPNKNITFKPSS
GGDLEITTHSFNCRGEFFYCNTSGLFSINYTENNTDGTPITLPCRIQRQ
IINMWQEVGRAMYAPPIEGNIACKSDITGLLLVRDGGSTNDSTNNNTEI
FRPAGGDMRDNWRSELYKYKVVEIKPLGIAPTEAKRRVVEREKR

In some embodiments, the recombinant gp120 protein comprises or consists essentially of an amino acid sequence at least 90% (such as at least 95%, at least 96%, at least 97%, at least 98% or at least 99%) identical to any one of SEQ ID NOs: 2-4 that comprises the V1 deletion of residues 137-152 (HXBc2 numbering).

In some embodiments, the immunogen comprises a gp160 or a HIV-1 Env trimer comprising the recombinant gp120 protein with the deletion of residues 137-152 (HXBc2 numbering. In some embodiments, the gp160 or the protomers of the HIV-1 Env trimer comprise or consist essentially of the amino acid sequence set forth as any one of:

SEQ ID NO: 5. HIV-1 A244 Env with V1 137-152
deletion and without signal peptide. Deletion
boundaries are underlined.
NLWKWGTLILGLVIICSASDNLWVTVYYGVPVWRDADTTLFCASDAKAQ
ETEAHNVWATHACVPTDPNPQELHLENVTENFNMWKNNMVEQMQEDVIS
LWDQSLKPCVKLTPLCVTLNCTNANLEVRNCSFNMTTELRDKKQKVHAL
FYKLDIVPIEDNTSSSKYRLINCNTSVIKQACPKISFDPIPIHYCTPAG
YAILKCNDKNFNGTGPCKNVSSVQCTHGIKPAVSTQLLINGSLAEEEII
IRSENLTNNAKTIIVHLNKSVEINCTRPSNNTRTSINIGPGQVFYRTGD
IIGDIRKAYCEINGAKWNEVLKKVTEKLKEHFNNKTIIFQPPSGGDLEI
TMHHFNCRGEFFYCNTTRLFNNTCMENETMEGCNGTIILPCKIKQIINM
WQRAGQAMYAPPISGRINCVSNITGILLTRDGGLNNTNETFRPGGGNIK
DNWRSELYKYKVVQIEPLGIAPTRAKRRVVEREKRAVGIGAMIFGFLGA
AGSTMGAASITLTVQARQLLSGIVQQQSNLLRAIEAQQHLLQLTVWGIK
QLQARVLAVERYLKDQKLLGLWGCSGKIICTTAVPWNSTWSNRSFEEIW
NNMTWIEWEREISNYTNQIYEILTQSQNQQDRNEKDLLELDKWASLWKW
FDITNWLWYIKIFIMIVGGLIGLRIIFAVLSIVNRVRQGYSPLSLQIPT
HHQREPDRPERIEEGGGEQGRDKSVRLVSGFLALTWDDLRSLCLFSYHR
LRDFISIAARTVELLGHSSLKGLRRGWEGLKYLGNLILYWGQELKISAI
SLLNATAIAVAGWTDRVIEVAQGAWRAILHIPRRIRQGLERTLL
SEQ ID NO: 6. HIV-1 MN Env with V1 137-152
deletion and without signal peptide. Deletion
boundaries are underlined.
HWWGWGTMLLGLLMICSATEKLWVTVYYGVPVWKEATTTLFCASDAKAY
DTEVHNVWATHACVPTDPNPQEVQLVNVTEDFNMWKNNMVEQMHEDIIS
LWDQSLKPCVKLTPLCVTLNCTDLRNEMKNCSFNITTSIRDKMQKEYAL
LYKLDIVAIDKDNTSYRLISCNTSVITQACPKVSFEPIPIHYCAPAGFA
ILKCNDKNFTGKGPCKNVSTVQCTHGIRPVVSTQLLINGSLAEEEVVIR
SENFTNNAKTIIVHLNESVQINCTRPYNNRRTRIHIGPGRAFYTTKNIK
GTIRQAHCTISSAKWNDTLRQIVSKLKEQFKNKTIVFKQSSGGDPEIVM
HSFNCGGEFFYCNTSSLFNSTWNGNNTWNNTTGSNSNITLQCKIKQIIN
MWQEVGKAMYAPPIEGQIRCSSNITGLLLTRDGGNDTDTNNTEIFRPGG
GDMRDNWRSELYKYKVVTIEPLGVAPTKAKRRVVQREKRAAIGALFLGF
LGAAGSTMGAASMMLTVQARQLLSGIVQQQNNLLRAIEAQQHMLQLTVW
GIKQLQARVLAVERYLRDQQLLGIWGCSGKLICTTTVPWNASWSNKSQE
DIWNNMTWMQWEREIDNYTSTIYELLEKSQNQQEKNEQELLELDKWASL
WNWFDITNWLWYIKIFIMIVGGLIGLRIVFAVLSIVNRVRQGYSPLSLQ
TRPPVPRGPDRPEGTEEEGGERDRDTSGRLVDGFLAIIWVDLRSLLLFS
YHRLRDLLLIAARIVELLGRRGWEILKYWWNLLQYWSQELKNSAVSLLN
ATAVAVAEGTDRVIEVLQRAGRAILHIPTRIRQGLERALL
SEQ ID NO: 7. HIV-1 96ZM651 Env with V1
137-152 deletion and without signal peptide.
Deletion boundaries are underlined.
RWWTWGILGFWMLMICNVWGNLWVTVYYGVPVWKEAKTTLFCASDAKSY
EKEVHNVWATHACVPTDPNPQEIVLGNVTENFNMWKNDMVDQMHEDIIS
LWDQSLKPCVKLTPLCVTLNCTEVNVDMKNCSFNITTELKDKKKNVYAL
FYKLDIVSLNETDDSETGNSSKYYRLINCNTSALTQACPKVSFDPIPIH
YCAPAGYAILKCNNKTFNGTGPCHNVSTVQCTHGIKPVVSTQLLLNGSL
AEEGIIIRSENLINNVKTIIVHLNRSIEIVCVRPNNNTRQSIRIGPGQT
FYATGDIIGDIRQAHCNISRTNWTKTLREVRNKLREHFPNKNITFKPSS
GGDLEITTHSFNCRGEFFYCNTSGLFSINYTENNTDGTPITLPCRIRQI
INMWQEVGRAMYAPPIEGNIACKSDITGLLLVRDGGSTNDSTNNNTEIF
RPAGGDMRDNWRSELYKYKVVEIKPLGTAPTEAKRRVVEREKRAVGIGA
VFLGFLGAAGSTMGAASITLTAQARQVLSGIVQQQSNLLRAIEAQQHLL
QLTVWGIKQLQTRVLAIERYLKDQQLLGLWGCSGKLICTTAVPWNISWS
NKSKTDIWDNMTWMQWDREISNYTNTIYRLLEDSQSQQEQNEKDLLALD
SWNNLWNWFDITKWLWYIKIFIMIVGGLIGLRIIFAVLSIVNRVRQGYS
PLSFQTLIPNPREPDRPGRIEEEGGEQDKERSVRLVSGFLALAWDDLRS
LCLFSYHRLRDFILVTARAVELLRRSSLKGLQRGWEALKYLGSLVQYWG
LELKKSAISLLDTIAIAVAEGTDRIIELIQGICRAIRNVPRRIRQGFET
ALL

In some embodiments, the recombinant gp160 or the protomers of the HIV-1 Env trimer comprise or consist essentially of an amino acid sequence at least 90% (such as at least 95%, at least 96%, at least 97%, at least 98% or at least 99%) identical to any one of SEQ ID NOs: 5-7 that comprises the V1 deletion of residues 137-152 (HXBc2 numbering).

The full-length sequences of A244 Env and HIV-1 Env clade B with the V1 137-152 deletion are provided as:

SEQ ID NO: 8. HIV-1 A244 Env with the V1 137-152
deletion. Deletion boundaries are underlined.
MRVKETQMTWPNLWKWGTLILGLVIICSASDNLWVTVYYGVPVWRDADT
TLFCASDAKAQETEAHNVWATHACVPTDPNPQELHLENVTENFNMWKNN
MVEQMQEDVISLWDQSLKPCVKLTPLCVTLNCTNANLEVRNCSFNMTTE
LRDKKQKVHALFYKLDIVPIEDNTSSSKYRLINCNTSVIKQACPKISFD
PIPIHYCTPAGYAILKCNDKNFNGTGPCKNVSSVQCTHGIKPAVSTQLL
LNGSLAEEEIIIRSENLINNAKTIIVHLNKSVEINCTRPSNNTRTSINI
GPGQVFYRTGDIIGDIRKAYCEINGAKWNEVLKKVTEKLKEHFNNKTII
FQPPSGGDLEITMHHFNCRGEFFYCNTTRLFNNTCMENETMEGCNGTII
LPCKIKQIINMWQRAGQAMYAPPISGRINCVSNITGILLTRDGGLNNTN
ETFRPGGGNIKDNWRSELYKYKVVQIEPLGIAPTRAKRRVVEREKRAVG
IGAMIFGFLGAAGSTMGAASITLTVQARQLLSGIVQQQSNLLRAIEAQQ
HLLQLTVWGIKQLQARVLAVERYLKDQKLLGLWGCSGKIICTTAVPWNS
TWSNRSFEEIWNNMTWIEWEREISNYTNQIYEILTQSQNQQDRNEKDLL
ELDKWASLWKWFDITNWLWYIKIFIMIVGGLIGLRIIFAVLSIVNRVRQ
GYSPLSLQIPTHHQREPDRPERIEEGGGEQGRDKSVRLVSGFLALTWDD
LRSLCLFSYHRLRDFISIAARTVELLGHSSLKGLRRGWEGLKYLGNLIL
YWGQELKISAISLLNATAIAVAGWTDRVIEVAQGAWRAILHIPRRIRQG
LERTLL
SEQ ID NO: 9. HIV-1 MN Env with the V1 137-152
deletion. Deletion boundaries are underlined.
MRVKGIRRNYQHWWGWGTMLLGLLMICSATEKLWVTVYYGVPVWKEATT
TLFCASDAKAYDTEVHNVWATHACVPTDPNPQEVQLVNVTEDFNMWKNN
MVEQMHEDIISLWDQSLKPCVKLTPLCVTLNCTDLRNEMKNCSFNITTS
IRDKMQKEYALLYKLDIVAIDKDNTSYRLISCNTSVITQACPKVSFEPI
PIHYCAPAGFAILKCNDKNFTGKGPCKNVSTVQCTHGIRPVVSTQLLLN
GSLAEEEVVIRSENFTNNAKTIIVHLNESVQINCTRPYNNRRTRIHIGP
GRAFYTTKNIKGTIRQAHCTISSAKWNDTLRQIVSKLKEQFKNKTIVFK
QSSGGDPEIVMHSFNCGGEFFYCNTSSLFNSTWNGNNTWNNTTGSNSNI
TLQCKIKQIINMWQEVGKAMYAPPIEGQIRCSSNITGLLLTRDGGNDTD
TNNTEIFRPGGGDMRDNWRSELYKYKVVTIEPLGVAPTKAKRRVVQREK
RAAIGALFLGFLGAAGSTMGAASMMLTVQARQLLSGIVQQQNNLLRAIE
AQQHMLQLTVWGIKQLQARVLAVERYLRDQQLLGIWGCSGKLICTTTVP
WNASWSNKSQEDIWNNMTWMQWEREIDNYTSTIYELLEKSQNQQEKNEQ
ELLELDKWASLWNWFDITNWLWYIKIFIMIVGGLIGLRIVFAVLSIVNR
VRQGYSPLSLQTRPPVPRGPDRPEGTEEEGGERDRDTSGRLVDGFLAII
WVDLRSLLLFSYHRLRDLLLIAARIVELLGRRGWEILKYWWNLLQYWSQ
ELKNSAVSLLNATAVAVAEGTDRVIEVLQRAGRAILHIPTRIRQGLERA
LL
SEQ ID NO: 10. HIV-1 96ZM651 Env strain with are
V1 137-152 deletion. Deletion boundaries
underlined.
MRVREILRNWQRWWTWGILGFWMLMICNVWGNLWVTVYYGVPVWKEAKT
TLFCASDAKSYEKEVHNVWATHACVPTDPNPQEIVLGNVTENFNMWKND
MVDQMHEDIISLWDQSLKPCVKLTPLCVTLNCTEVNVDMKNCSFNITTE
LKDKKKNVYALFYKLDIVSLNETDDSETGNSSKYYRLINCNTSALTQAC
PKVSFDPIPIHYCAPAGYAILKCNNKTFNGTGPCHNVSTVQCTHGIKPV
VSTQLLLNGSLAEEGIIIRSENLINNVKTIIVHLNRSIEIVCVRPNNNT
RQSIRIGPGQTFYATGDIIGDIRQAHCNISRTNWTKTLREVRNKLREHF
PNKNITFKPSSGGDLEITTHSFNCRGEFFYCNTSGLFSINYTENNTDGT
PITLPCRIRQIINMWQEVGRAMYAPPIEGNIACKSDITGLLLVRDGGSI
NDSINNNTEIFRPAGGDMRDNWRSELYKYKVVEIKPLGIAPTEAKRRVV
EREKRAVGIGAVFLGFLGAAGSTMGAASITLTAQARQVLSGIVQQQSNL
LRAIEAQQHLLQLTVWGIKQLQTRVLAIERYLKDQQLLGLWGCSGKLIC
TTAVPWNISWSNKSKTDIWDNMTWMQWDREISNYTNTIYRLLEDSQSQQ
EQNEKDLLALDSWNNLWNWFDITKWLWYIKIFIMIVGGLIGLRIIFAVL
SIVNRVRQGYSPLSFQTLIPNPREPDRPGRIEEEGGEQDKERSVRLVSG
FLALAWDDLRSLCLFSYHRLRDFILVTARAVELLRRSSLKGLQRGWEAL
KYLGSLVQYWGLELKKSAISLLDTIAIAVAEGTDRIIELIQGICRAIRN
VPRRIRQGFETALL

In some embodiments, the recombinant gp120, gp140, gp145, gp160, or the protomers of the recombinant HIV-1 Env ectodomain trimer can further include a non-natural disulfide bond between HIV-1 Env positions 201 and 433. For example, the non-natural disulfide bond can be introduced by including cysteine substitutions at positions 201 and 433 (e.g., I201C and A433C substitutions). The presence of the non-natural disulfide bond between residues 201 and 433 contributes to the stabilization of the HIV-1 Env protein in its prefusion mature closed conformation.

In some embodiments, the protomers of the recombinant HIV-1 Env ectodomain trimer can include gp120-gp41 ectodomain protomers further including the “SOSIP” substitutions, which include a non-natural disulfide bond between cysteine residues introduced at HIV-1 Env positions 501 and 605 (for example, by A501C and T605C substitutions), and a proline residue introduced at HIV-1 Env positions 559 (for example, by an I559P substitution). The presence of the non-natural disulfide bond between positions 501 and 605 and the proline residue at position 559 contributes to the stabilization of the HIV-1 Env ectodomain in the prefusion mature closed conformation. In several embodiments, the protomers of the recombinant HIV-1 Env ectodomain trimer can further include a non-natural disulfide bond between HIV-1 Env positions 201 and 433 (e.g., by introduction of I201C and A433C substitutions) and the HIV-1 Env ectodomain trimer can further include the SOSIP mutations.

In some embodiments, the recombinant gp120, gp140, gp145, gp160, or the protomers of the recombinant HIV-1 Env ectodomain trimer can further include an N-linked glycosylation site at HIV-1 Env position 332 (if not already present on the ectodomain). For example, by T332N substitution in the case of BG505-based immunogens. The presence of the glycosylation site at N332 allows for binding by 2G12 antibody.

In some embodiments, the recombinant gp120, gp140, gp145, gp160, or the protomers of the recombinant HIV-1 Env ectodomain trimer can include a lysine residue at HIV-1 Env position 168 (if not already present on the ectodomain). For example, the lysine residue can be added by amino acid substitution (such as an E168K substitution in the case of the JR-FL based immunogens). The presence of the lysine residue at position 168 allows for binding of particular broadly neutralizing antibodies to the V1V2 loops of gp120.

In some embodiments, the protomers of the recombinant HIV-1 Env ectodomain trimer can further include mutations to add an N-linked glycan sequon at position 504, position 661, or positions 504 and 661, to increase glycosylation of the membrane proximal region of the ectodomain.

Native HIV-1 Env sequences include a furin cleavage site between positions 508 and 512 (HXBc2 numbering), that separates gp120 and gp41. Any of the disclosed recombinant gp160 proteins and HIV-1 Env ectodomains can further include an enhanced cleavage site between gp120 and gp41 proteins. The enhanced cleavage cite can include, for example, substitution of six arginine resides for the four residues of the native cleavage site (e.g., REKR, SEQ ID NO: 11) to RRRRRR (SEQ ID NO: 12). It will be understood that protease cleavage of the furin or enhanced cleavage site separating gp120 and gp41 can remove a few amino acids from either end of the cleavage site.

The recombinant HIV-1 Env ectodomain trimer includes a protein complex of gp120-gp41 ectodomain protomers. The gp120 -gp41 ectodomain protomer can include separate gp120 and gp41 polypeptide chains, or can include gp120 and gp41 polypeptide chains that are linked (e.g., by a peptide linker) to form a single polypeptide chain (e.g., a “single chain”). In several embodiments, the recombinant HIV-1 Env ectodomain trimer is membrane anchored and can include a trimeric complex of recombinant HIV-1 Env ectodomains that are linked to a transmembrane domain (e.g., a gp145 protein including a gp120 protein and a gp41 ectodomain and transmembrane domain).

In several embodiments, the N-terminal residue of the recombinant gp120 protein is one of HIV-1 Env positions 1-35, and the C-terminal residue of the recombinant gp120 protein is one of HIV-1 Env positions 503-511. In some embodiments, the N-terminal residue of the recombinant gp120 protein is HIV-1 Env position 31 and the C-terminal residue of the recombinant gp120 protein is HIV-1 Env position 511 or position 507. In some embodiments, the recombinant gp120 protein comprises or consists of HIV-1 Env positions 31-507 (HXBc2 numbering).

The purified proteins provided herein typically do not include a signal peptide (for example, the purified recombinant gp120 protein typically does not include HIV-1 Env positions 1-30), as the signal peptide is proteolytically cleaved during cellular processing.

In embodiments including a soluble recombinant HIV-1 Env ectodomain, the gp41 ectodomain is not linked to a transmembrane domain or other membrane anchor. However, in embodiments including a membrane anchored recombinant HIV-1 Env ectodomain trimer the gp41 ectodomain can be linked to a transmembrane domain (such as, but not limited to, an HIV-1 Env transmembrane domain).

In some embodiments, the HIV-1 Env ectodomain trimer includes the recombinant gp120 protein and the gp41 ectodomain, wherein the N-terminal residue of the recombinant gp120 protein is HIV-1 Env position 31; the C-terminal residue of the recombinant gp120 protein is HIV-1 Env position 507 or 511; the N-terminal residue of the gp41 ectodomain is HIV-1 Env position 512; and the C-terminal residue of the gp41 ectodomain is HIV-1 Env position 664. In some embodiments, the HIV-1 Env ectodomain trimer includes the recombinant gp120 protein and the gp41 ectodomain, wherein the N-terminal residue of the recombinant gp120 protein is HIV-1 Env position 31; the C-terminal residue of the recombinant gp120 protein is HIV-1 Env position 507; the N-terminal residue of the gp41 ectodomain is HIV-1 Env position 512; and the C-terminal residue of the gp41 ectodomain is HIV-1 Env position 664. In some embodiments, the C-terminal residue of the recombinant HIV-1 Env ectodomain is position 683 (the entire ectodomain, terminating just before the transmembrane domain). In additional embodiments, the C-terminal residue of the recombinant HIV-1 Env ectodomain is position 707 (the entire ectodomain, terminating just after the transmembrane domain).

In view of the conservation and breadth of knowledge of HIV-1 Env sequences, the person of ordinary skill in the art can easily identify corresponding HIV-1 Env amino acid positions between different HIV-1 Env strains and subtypes. The HXBc2 numbering system has been developed to assist comparison between different HIV-1 amino acid and nucleic acid sequences. The numbering of amino acid substitutions disclosed herein is made according to the HXBc2 numbering system, unless context indicates otherwise.

It is understood in the art that some variations can be made in the amino acid sequence of a protein without affecting the activity of the protein. Such variations include insertion of amino acid residues, deletions of amino acid residues, and substitutions of amino acid residues. These variations in sequence can be naturally occurring variations or they can be engineered through the use of genetic engineering technique known to those skilled in the art. Examples of such techniques are found in see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, 2013, both of which are incorporated herein by reference in their entirety.

The recombinant gp120, gp140, gp145, gp160, or the protomers of the recombinant HIV-1 Env ectodomain trimer can be derivatized or linked to another molecule (such as another peptide or protein). In general, the derivatization is such that the binding of antibodies that bind to the V2 domain (or of the V2b or V2c peptides disclosed herein) is not affected adversely by the derivatization or labeling. In some embodiments, the recombinant gp120, gp140, gp145, gp160, or the protomers of the recombinant HIV-1 Env ectodomain trimer can be functionally linked (by chemical coupling, genetic fusion, noncovalent association or otherwise) to one or more other molecular entities, such as an antibody or protein or detection tag.

### Membrane Anchored Embodiments

In some embodiments, the HIV-1 Env ectodomain trimer including the recombinant gp120 protein can be a membrane anchored HIV-1 Env ectodomain trimer, for example, the HIV-1 Env ectodomains in the trimer can each be linked to a transmembrane domain. The transmembrane domain can be linked to any portion of the HIV-1 Env ectodomain, as long as the presence of the transmembrane domain does not disrupt the structure of the HIV-1 Env ectodomain, or its ability to induce an immune response to HIV-1. In non-limiting examples, the transmembrane domain can be linked to the N- or C-terminal residue of a gp120 polypeptide, or the C-terminal residue of a gp41 ectodomain included in the HIV-1 Env ectodomain. One or more peptide linkers (such as a gly-ser linker, for example, a 10 amino acid glycine-serine peptide linker, such as a peptide linker comprising the amino acid sequence set forth as SEQ ID NO: 13 (GGSGGGGSGG) can be used to link the transmembrane domain and the gp120 or gp41 protein. In some embodiments a native HIV-1 Env MPER sequence can be used to link the transmembrane domain and the gp120 or gp41 protein.

Non-limiting examples of transmembrane domains for use with the disclosed embodiments include the BG505 TM domain (KIFIMIVGGLIGLRIVFAVLSVIHRVR, SEQ ID NO: 14), the Influenza A Hemagglutinin TM domain (ILAIYSTVASSLVLLVSLGAISF, SEQ ID NO: 15), and the Influenza A Neuraminidase TM domain (IITIGSICMVVGIISLILQIGNIISIWVS, SEQ ID NO: 16).

The recombinant HIV-1 Env ectodomain linked to the transmembrane domain can include any of the mutations provided herein (or combinations thereof) as long as the recombinant HIV-1 Env ectodomain linked to the transmembrane domain retains the desired properties.

### Linkage to a Trimerization Domain

In several embodiments, the HIV-1 Env ectodomain trimer including the recombinant gp120 protein can be linked to a trimerization domain, for example, the C-terminus of the gp41 ectodomains included in the HIV-1 Env ectodomain trimer can be linked to the trimerization domain. The trimerization domain can promote trimerization of the three protomers of the recombinant HIV-1 Env protein. Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to the recombinant HIV-1 Env ectodomain (e.g., by linkage to the C-terminus of the gp41 polypeptide to promote trimerization of the recombinant HIV-1 protein.

In some examples, the recombinant HIV-1 Env ectodomain can be linked to a T4 fibritin Foldon domain, for example, the recombinant HIV-1 Env ectodomain can include a gp41 polypeptide with a Foldon domain linked to its C-terminus. In specific examples, the T4 fibritin Foldon domain can include the amino acid sequence GYIPEAPRDGQAYVRKDGEWVLLSTF (SEQ ID NO: 17), which adopts a β-propeller conformation, and can fold and trimerize in an autonomous way (Tao et al. 1997 Structure 5:789-798).

Typically, the heterologous trimerization domain is positioned C-terminal to the gp41 protein. Optionally, the heterologous trimerization is connected to the recombinant HIV-1 Env ectodomain via a linker, such as an amino acid linker. Exemplary linkers include Gly or Gly-Ser linkers, such as SEQ ID NO: 13 (GGSGGGGSGG). Some embodiments include a protease cleavage site for removing the trimerization domain from the HIV-1 polypeptide, such as, but not limited to, a thrombin site between the recombinant HIV-1 Env ectodomain and the trimerization domain.

### Carrier Molecules

In some embodiments, the recombinant gp120, gp140, gp145, gp160, or recombinant HIV-1 Env ectodomain trimer can be linked to a carrier protein by a linker (such as a peptide linker) or can be directly linked to the carrier protein (for example, by conjugation, or synthesis as a fusion protein) too form an immunogenic conjugate.

Suitable linkers are well known to those of skill in the art and include, but are not limited to, straight or branched-chain carbon linkers, heterocyclic carbon linkers or peptide linkers. One skilled in the art will recognize, for an immunogenic conjugate from two or more constituents, each of the constituents will contain the necessary reactive groups. Representative combinations of such groups are amino with carboxyl to form amide linkages or carboxy with hydroxyl to form ester linkages or amino with alkyl halides to form alkylamino linkages or thiols with thiols to form disulfides or thiols with maleimides or alkylhalides to form thioethers. Hydroxyl, carboxyl, amino and other functionalities, where not present may be introduced by known methods. Likewise, as those skilled in the art will recognize, a wide variety of linking groups may be employed. In some cases, the linking group can be designed to be either hydrophilic or hydrophobic in order to enhance the desired binding characteristics of the HIV-1 Env protein and the carrier. The covalent linkages should be stable relative to the solution conditions under which the conjugate is subjected.

In some embodiments, the linkers may be joined to the constituent amino acids through their side groups (such as through a disulfide linkage to cysteine) or to the alpha carbon amino and carboxyl groups of the terminal amino acids. In some embodiments, the recombinant gp120, gp140, gp145, gp160, or the protomers of the recombinant HIV-1 Env ectodomain trimer, the linker, and the carrier can be encoded as a single fusion polypeptide such that the recombinant gp120, gp140, gp145, gp160, or the protomers of the recombinant HIV-1 Env ectodomain trimer and the carrier are joined by peptide bonds.

The procedure for attaching a molecule to a polypeptide varies according to the chemical structure of the molecule. Polypeptides typically contain a variety of functional groups; for example, carboxylic acid (COOH), free amine (—NH₂) or sulfhydryl (—SH) groups, which are available for reaction with a suitable functional group on a polypeptide. Alternatively, the polypeptide is derivatized to expose or attach additional reactive functional groups. The derivatization may involve attachment of any of a number of linker molecules such as those available from Pierce Chemical Company, Rockford, IL.

It can be advantageous to produce conjugates in which more than one recombinant gp120, gp140, gp145, gp160, or HIV-1 Env ectodomain trimer is conjugated to a single carrier protein. In several embodiments, the conjugation of multiple recombinant gp120, gp140, gp145, gp160, or HIV-1 Env ectodomain trimers to a single carrier protein is possible because the carrier protein has multiple lysine or cysteine side-chains that can serve as sites of attachment.

Examples of suitable carriers are those that can increase the immunogenicity of the conjugate and/or elicit antibodies against the carrier which are diagnostically, analytically, and/or therapeutically beneficial. Useful carriers include polymeric carriers, which can be natural, recombinantly produced, semi-synthetic or synthetic materials containing one or more amino groups, such as those present in a lysine amino acid residue present in the carrier, to which a reactant moiety can be attached. Carriers that fulfill these criteria are generally known in the art (see, for example, Fattom et al., Infect. Immun. 58:2309-12, 1990; Devi et al., PNAS 88:7175-79, 1991; Szu et al., Infect. Immun. 59:4555-61, 1991; Szu et al., J. Exp. Med. 166:1510-24, 1987; and Pavliakova et al., Infect. Immun. 68:2161-66, 2000). A carrier can be useful even if the antibody that it elicits is not of benefit by itself.

Specific, non-limiting examples of suitable polypeptide carriers include, but are not limited to, natural, semi-synthetic or synthetic polypeptides or proteins from bacteria or viruses. In one embodiment, bacterial products for use as carriers include bacterial toxins. Bacterial toxins include bacterial products that mediate toxic effects, inflammatory responses, stress, shock, chronic sequelae, or mortality in a susceptible host. Specific, non-limiting examples of bacterial toxins include, but are not limited to: B. anthracis PA (for example, as encoded by bases 143779 to 146073 of GENBANK® Accession No. NC 007322); B. anthracis LF (for example, as encoded by the complement of bases 149357 to 151786 of GENBANK® Accession No. NC 007322); bacterial toxins and toxoids, such as tetanus toxin/toxoid (for example, as described in U.S. Pat. Nos. 5,601,826 and 6,696,065); diphtheria toxin/toxoid (for example, as described in U.S. Pat. Nos. 4,709,017 and 6,696,065), such as tetanus toxin heavy chain C fragment; P. aeruginosa exotoxin/toxoid (for example, as described in U.S. Pat. Nos. 4,428,931, 4,488,991 and 5,602,095); pertussis toxin/toxoid (for example, as described in U.S. Pat. Nos. 4,997,915, 6,399,076 and 6,696,065); and C. perfringens exotoxin/toxoid (for example, as described in U.S. Pat. Nos. 5,817,317 and 6,403,094) C. difficile toxin B or A, or analogs or mimetics of and combinations of two or more thereof. Viral proteins, such as hepatitis B surface antigen (for example, as described in U.S. Pat. Nos. 5,151,023 and 6,013,264) and core antigen (for example, as described in U.S. Pat. Nos. 4,547,367 and 4,547,368) can also be used as carriers, as well as proteins from higher organisms such as keyhole limpet hemocyanin (KLH), horseshoe crab hemocyanin, Concholepas Concholepas Hemocyanin (CCH), Ovalbumin (OVA), edestin, mammalian serum albumins (such as bovine serum albumin), and mammalian immunoglobulins. In some examples, the carrier is bovine serum albumin.

In some embodiments, the carrier is selected from one of: Keyhole Limpet Hemocyanin (KLH), tetanus toxoid, tetanus toxin heavy chain C fragment, diphtheria toxoid, diphtheria toxin variant CRM197, or H influenza protein D (HiD). CRM197 is a genetically detoxified form of diphtheria toxin; a single mutation at position 52, substituting glutamic acid for glycine, causes the ADP-ribosyltransferase activity of the native diphtheria toxin to be lost. For description of protein carriers for vaccines, see Pichichero, Protein carriers of conjugate vaccines: characteristics, development, and clinical trials, Hum Vaccin Immunother., 9: 2505-2523,2013, which is incorporated by reference herein in its entirety).

Following conjugation of the recombinant gp120, gp140, gp145, gp160, or HIV-1 Env ectodomain trimer to the carrier protein, the conjugate can be purified by a variety of techniques well known to one of skill in the art. The conjugates can be purified away from unconjugated material by any number of standard techniques including, for example, size exclusion chromatography, density gradient centrifugation, hydrophobic interaction chromatography, or ammonium sulfate fractionation. See, for example, Anderson et al., J. Immunol. 137:1181-86, 1986 and Jennings & Lugowski, J. Immunol. 127:1011-18, 1981. The compositions and purity of the conjugates can be determined by GLC-MS and MALDI-TOF spectrometry, for example.

In several embodiments, the disclosed immunogenic conjugates can be formulated into immunogenic composition (such as vaccines), for example by the addition of a pharmaceutically acceptable carrier and/or adjuvant.

### B. Polynucleotides and Expression

Polynucleotides encoding a disclosed immunogen are also of use in the disclosed methods. These polynucleotides include DNA, cDNA and RNA sequences which encode the antigen. One of skill in the art can readily use the genetic code to construct a variety of functionally equivalent nucleic acids, such as nucleic acids which differ in sequence but which encode the same protein sequence, or encode a conjugate or fusion protein including the nucleic acid sequence.

For example, in some embodiments, the polynucleotide encodes a V1 deleted HIV-1 Env sequence, for example, the polynucleotide comprises the DNA sequence set forth as:

A244 gp160 V1 deleted
(SEQ ID NO: 18)
ATGAGAGTGAAGGAGACACAGATGAATTGGCCAAACTTGTGGAAATGGG
GGACTTTGATCCTTGGGTTGGTGATAATTTGTAGTGCCTCAGACAACTT
GTGGGTTACAGTTTATTATGGGGTTCCTGTGTGGAGAGATGCAGATACC
ACCCTATTTTGTGCATCAGATGCCAAAGCACATGAGACAGAAGTGCACA
ATGTCTGGGCCACACATGCCTGTGTACCCACAGACCCCAACCCACAAGA
AATAGACCTGGAAAATGTAACAGAAAATTTTAACATGTGGAAAAATAAC
ATGGTAGAGCAGATGCAGGAGGATGTAATCAGTTTATGGGATCAAAGTC
TAAAGCCATGTGTAAAGTTAACTCCTCTCTGCGTTACTTTACATTGTAC
TAATGCTAATTTGGAAGTAAGAAACTGTTCTTTTAATATGACCACAGAA
CTAAGAGATAAGAAGCAGAAGGTCCATGCACTTTTTTATAAGCTTGATA
TAGTACCAATTGAAGATAATAACGATAATAGTAAGTATAGGTTAATAAA
TTGTAATACTTCAGTCATTAAGCAGGCTTGTCCAAAGATATCCTTTGAT
CCAATTCCTATACATTATTGTACTCCAGCTGGTTATGCGATTTTAAAGT
GTAATGATAAGAATTTCAATGGGACAGGGCCATGTAAAAACGTCAGCTC
AGTACAATGCACACATGGAATTAAGCCAGTGGTATCAACTCAATTGCTG
TTAAATGGCAGTCTAGCAGAAGAAGAGATAATAATCAGATCTGAAGATC
TCACAAACAATGCCAAAACCATAATAGTGCACCTTAATAAATCTGTAGT
AATCAATTGTACCAGACCCTCCAACAATACAAGAACAAGTATAACTATA
GGACCAGGACAAGTATTCTATAGAACAGGAGACATAATAGGAGATATAA
GAAAAGCATATTGTGAGATTAATGGAACAGAATGGAATAAAGCTTTAAA
ACAGGTAACTGAAAAGTTAAAAGAGCACTTTAATAATAAGCCAATAATC
TTTCAACCACCCTCAGGAGGAGATCTAGAAATTACAATGCATCATTTTA
ATTGTAGAGGAGAATTTTTCTATTGCAATACAACACGACTGTTTAATAA
TACTTGCATAGCAAATGGAACCATAGAGGGGTGTAATGGCAATATCACA
CTTCCATGCAAGATAAAACAAATTATAAACATGTGGCAGGGAGCAGGAC
AAGCAATGTATGCTCCTCCCATCAGTGGAACAATTAATTGTGTATCAAA
TATTACAGGAATACTATTGACAAGAGATGGTGGTGCTACTAATAATACG
AATAACGAGACCTTCAGACCTGGAGGAGGAAATATAAAGGACAATTGGA
GAAATGAATTATATAAATATAAAGTAGTACAAATTGAACCACTAGGAGC
AGCACCCACCAGGGCAAAGAGAAGAGTGGTGGAGAGAGAAAAAAGAGCA
GTGGGAATAGGAGCTATGATCTTTGGGTTCTTAGGAGCAGCAGGAAGCA
CTATGGGCGCGGCGTCAATAACGCTGACGGTACAGGCCAGACAATTATT
GTCTGGTATAGTGCAACAGCAAAGCAATTTGCTGAGGGCTATAGAGGCG
CAGCAGCATCTGTTGCAACTCACAGTCTGGGGCATTAAACAGCTCCAGG
CAAGAGTCCTGGCTGTGGAAAGATACCTAAAGGATCAAAAGTTCCTAGG
ACTTTGGGGCTGCTCTGGAAAAATCATCTGCACCACTGCAGTGCCCTGG
AACTCCACTTGGAGTAATAAATCTCTTGAAGAGATTTGGAACAACATGA
CATGGATAGAATGGGAGAGAGAAATTAGCAATTACACAAACCAAATATA
TGAGATACTTACAAAATCGCAGGACCAGCAGGACAGGAATGAAAAGGAT
TTGTTAGAATTGGATAAATGGGCAAGTCTGTGGACTTGGTTTGACATAA
CAAATTGGCTGTGGTATATAAAAATATTTATAATGATAGTGGGAGGTTT
AATAGGATTAAGAATAATTTTTGCTGTGCTTTCTATAGTGAATAGAGTT
AGGCAGGGATACTCACCTTTGTCTTTCCAGACCCCTTGCCATCATCAGA
GGGAACCCGACAGACCCGAAAGAATCGAAGAAGAAGGTGGCGAGCAAGG
CAGAGACAGATCCGTGCGATTAGTGAGCGGATTCTTAGCTCTTGCATGG
GACGATCTACGGAGCCTGTGCCTCTTCAGCTACCACCGCTTGAGAGACT
TCATCTTGATTGCAGCGAGGACTGTGGAACTTCTGGGACGCAGCAGTCT
CAAGGGACTGAGACGGGGGTGGGAAGGCCTCAAATATCTGGGGAATCTT
CTGTTATATTGGGGTCAGGAACTAAAAATTAGTGCTATTTCTTTGCTTG
ATGCTACAGCAATAGCAGTAGCGGGGTGGACAGATAGGGTTATAGAAGT
AGCACAAGGAGCTTGGAAAGCCATTCTCCACATACCTAGAAGAATCAGA
CAGGGCTTAGAAAGGGCTTTGCAATAA

In several embodiments, the nucleic acid molecule encodes a precursor of a protomer of a disclosed HIV-1 Env trimer, that, when expressed in cells under appropriate conditions, forms HIV-1 Env trimers and is processed into the mature form of the HIV-1 Env protein.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed., Cold Spring Harbor, New York, 2012) and Ausubel et al. (In Current Protocols in Molecular Biology, John Wiley & Sons, New York, 2013). Product information from manufacturers of biological reagents and experimental equipment also provide useful information. Such manufacturers include the SIGMA Chemical Company (Saint Louis, MO), R&D Systems (Minneapolis, MN), Pharmacia Amersham (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersburg, MD), Fluka Chemica-Biochemika Analytika (Fluka Chemie AG, Buchs, Switzerland), Invitrogen (Carlsbad, CA), and Applied Biosystems (Foster City, CA), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

Polynucleotides encoding an immunogen can include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding an immunogen can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the immunogen can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). One of skill in the art can readily use expression systems such as plasmids and vectors of use in producing proteins in cells including higher eukaryotic cells such as the COS, CHO, HeLa and myeloma cell lines.

In one non-limiting example, an immunogen is expressed using the pVRC8400 vector (described in Barouch et al., J. Virol, 79,8828-8834, 2005, which is incorporated by reference herein).

Modifications can be made to a nucleic acid encoding an immunogen without diminishing its biological activity. Some modifications can be made to facilitate the cloning, expression, or incorporation of the targeting molecule into a fusion protein. Such modifications are well known to those of skill in the art and include, for example, termination codons, a methionine added at the amino terminus to provide an initiation, site, additional amino acids placed on either terminus to create conveniently located restriction sites, or additional amino acids (such as poly His) to aid in purification steps.

### C. Viral Vectors

A nucleic acid molecule encoding an immunogen (e.g., a recombinant gp120 protein or a HIV-1 Env ectodomain trimer comprising the recombinant gp120 protein) can be included in a viral vector, for example, for expression of the immunogen in a host cell, or for immunization of a subject as disclosed herein. In some embodiments, the viral vectors are administered to a subject as part of a prime-boost vaccination. In several embodiments, the viral vectors are included in a vaccine, such as a primer vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

In several embodiments, the viral vector can include an adenoviral vector that expresses a disclosed recombinant HIV-1 Env ectodomain or immunogenic fragment thereof. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Replication competent and deficient adenoviral vectors (including singly and multiply replication deficient adenoviral vectors) can be used with the disclosed embodiments. Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Application Nos. WO 94/28152, WO 95/02697, WO 95/16772, WO 95/34671, WO 96/22378, WO 97/12986, WO 97/21826, and WO 03/02231 1.

### D. Virus-Like Particles

In some embodiments, a virus-like particle (VLP) that includes an immunogen (e.g., a recombinant HIV-1 Env ectodomain or immunogenic fragment thereof) is of use in the disclosed methods. VLPs lack the viral components that are required for virus replication and thus represent a highly attenuated, replication-incompetent form of a virus. However, the VLP can display a polypeptide (e.g., a recombinant HIV-1 Env protein) that is analogous to that expressed on infectious virus particles and should be equally capable of eliciting an immune response to HIV when administered to a subject. Virus like particles and methods of their production are known, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation. The virus like particle can include any recombinant gp120 proteins or recombinant HIV-1 Env ectodomain trimers or an immunogenic fragment thereof.

### E. Immunogenic Compositions

Immunogenic compositions comprising a disclosed immunogen and a pharmaceutically acceptable carrier are of use in the disclosed methods. Such compositions can be administered to subjects by a variety of administration modes, for example, intramuscular, subcutaneous, intravenous, intra-arterial, intra-articular, intraperitoneal, or parenteral routes. Methods for preparing administrable compositions are described in more detail in such publications as Remingtons Pharmaceutical Sciences, 19^{th} Ed., Mack Publishing Company, Easton, Pennsylvania, 1995.

Thus, an immunogen can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The pharmaceutical composition can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The pharmaceutical composition may optionally include an adjuvant to enhance an immune response of the host. Suitable adjuvants are, for example, toll-like receptor agonists, alum, A1PO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some embodiments, the composition can be provided as a sterile composition. The pharmaceutical composition typically contains an effective amount of the immunogen and can be prepared by conventional techniques. Typically, the amount of immunogen in each dose of the immunogenic composition is selected as an amount which elicits an immune response without significant, adverse side effects. In some embodiments, the composition can be provided in unit dosage form for use to elicit an immune response in a subject, for example, to prevent HIV-1 infection in the subject. A unit dosage form contains a suitable single preselected dosage for administration to a subject, or suitable marked or measured multiples of two or more preselected unit dosages, and/or a metering mechanism for administering the unit dose or multiples thereof. In other embodiments, the composition further includes an adjuvant.

### III. SAMT-247 Microbicide

The presently disclosed methods use an effective amount of a SAMT-247 microbicide. The compound SAMT-247 has a formula C₁₂H₁₄N₂O₃S, and the chemical structure of:

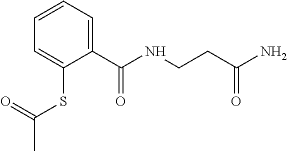 [SMILES: CC(=O)Sc1ccccc1C(=O)NCCC(N)=O]

An effective amount of a SAMT-247 microbicide can be provided as a pharmaceutically acceptable salt, and derivative, or a prodrug form of SAMT-247. A pharmaceutical composition comprising an effective amount of a SAMT-247 microbicide and a pharmaceutically acceptable carrier can be used in the disclosed methods. The pharmaceutically acceptable carrier can be any of those conventionally used and is limited only by chemico-physical considerations, such as solubility and lack of reactivity with the compound, and by the route of administration. It will be appreciated by one of skill in the art that, in addition to the following described pharmaceutical compositions can make formulations that include inclusion complexes, such as cyclodextrin inclusion complexes, or liposomes.

The pharmaceutically acceptable carriers described herein, for example, vehicles, adjuvants, excipients, or diluents, are well known to those who are skilled in the art and are readily available to the public. In some embodiments, the pharmaceutically acceptable carrier can be chemically inert to the active compound and one which has no detrimental side effects or toxicity under the conditions of use. The choice of carrier will be determined in part by the particular active agent, as well as by the particular method used to administer the composition. Accordingly, there is a wide variety of suitable formulations of the pharmaceutical composition. Formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art. Suitable doses for oral formulations include, such as for a SAMT-247 microbicide include, but are not limited to, about 100 to about 500 mg/kg, such as about 100, 200, 300, 400 or 500 mg/kg, for example about 300 mg/kg. Oral formulations can be administered daily, for example, for 1, 2, 3, 4, 5, 6, or 7 days.

An effective amount of a SAMT-247 microbicide can be used alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. An effective amount of the SAMT-247 microbicide can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

Injectable formulations can be produced that include an effective amount of the SAMT-247 microbicide for use in the disclosed methods. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topical formulations, including those that are useful for transdermal drug release, are well-known to those of skill in the art and are suitable in the context of the invention for application to skin. Generally, the effective amount of the SAMT-247 microbicide may be administered as a topical ointment applied to the lining of the vagina and/or cervix and/or rectum, which can be accomplished as a gel, cream, lotion, non-aqueous or aqueous solution used to flush the vaginal or rectal cavity, and/or a vaginal or rectal suppository. In other embodiments, the effective amount of the SAMT-247 microbicide may be administered in a spray formulation. In addition, the effective amount of the SAMT-247 microbicide may be delivered using microbicide-impregnated diaphragms and female and male condoms. In some embodiments, the composition contains at least an effective amount of the SAMT-247 microbicide and a suitable vehicle or carrier. It may also contain other components, such as an anti-irritant. An effective amount of the SAMT-247 microbicide can be delivered to the vagina of a mammal by any means known to those skilled in the art including gels, foams, intervaginal sponges and films.

In some embodiments, the composition includes a carrier. The carrier can be a liquid, solid or semi-solid. In embodiments, the composition is an aqueous solution. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. An effective amount of the SAMT-247 microbicide can be included in personal care products, such as, for example, condom lubricants, and the like. Such lubricants may comprise commonly known ingredients such as, for example: humectants, e.g., glycerin, sorbitol, mannitol, glycols and glycol ethers; buffers, e.g., glucono-d-lactone; germicides or bactericides, e.g., chlorhexidine gluconate; preservatives, e.g., methylparaben; viscosifiers, e.g., hydroxyethyl cellulose, etc.; other adjuvants, e.g., colors and fragrances; in addition to the compositions of the present disclosure. Those skilled in the art will recognize that the physical properties, e.g., viscosity, of such delivery forms may vary widely. For example, the viscosity of a gel form, e.g., about 150,000 centipoise, may be substantially higher than the viscosity of lotion form, e.g., about 100 centipoise. Further details concerning the materials, ingredients, proportions and procedures of such delivery forms can be selected in accordance with techniques well-known in the art.

The compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application can include water, buffered solutions, various alcohols, glycols such as glycerin, lipid materials such as fatty acids, mineral oils, phosphoglycerides, collagen, gelatin and silicone based materials.

In some embodiments, in addition to the effective amount of the SAMT-247 microbicide, the balance of the compositions, i.e., typically from about 0-10% weight, or from about 0.1-5% weight, or from about 0.1-3% weight, may optionally comprise one or more cosmetic ingredients. Such cosmetic ingredients can include diluents, solvents, and/or adjuvants. Typically, cosmetic ingredients include, for example; water, ethyl alcohol, isopropyl alcohol, glycerin, glycerol propylene glycol, sorbitol, and other high molecular weight alcohols. In addition, contraceptive compositions that include an effective amount of the SAMT-247 microbicide may contain minor amounts of other additives, such as, for example; stabilizers, surfactants, menthol, eucalyptus oil, other essential oils, fragrances, and the like. The selection and amounts of cosmetic ingredients, other additives, and blending procedures can be carried out in accordance with techniques well-known in the art.

An effective amount of a SAMT-247 microbicide can be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

In some embodiments, the composition is formulated for topical administration to the vagina of a female human before and/or after sexual intercourse. Other methods of topical administration are possible, such as administration to the penis (e.g., formulated as a lubricant), and may also depend on sexual practices.

In one embodiment, a contraceptive microbicide, see for example, U.S. Pat. No. 6,706,276, and PCT publication No. WO 01/66084), that is a gel that forms a matrix upon contact with ejaculate and thus entraps and inactivates spermatozoa and/or microbes. In some embodiments, the contraceptive microbicide contains (a) a matrix-forming compound, (b) a bioadhesive compound, and (c) lactic acid. Some compounds, such as chitosan, can act as both the matrix-forming compound and the bioadhesive compound. In exemplary embodiments, the contraceptive microbicide contains (1) about 1-10% of one or more matrix-forming compounds, (2) about 1-10% of one or more bioadhesive compounds, and (3) about 1-10% of lactic acid. In other embodiments, the composition contains (1) about 3-5% of one or more matrix-forming compounds, (2) about 2.5-6% of one or more bioadhesive compounds, and (3) about 1-7% of lactic acid. In other embodiments, the composition contains (1) about 3.5-4.5% of one or more matrix-forming compounds, (2) about 2.5-3.5% of one or more bioadhesive compounds, and (3) about 1-4% of lactic acid. An effective amount of SAMT-247 can be included in these compositions.

Matrix-forming compounds suitable for use in the methods of the present disclosure can be stable over a wide pH range, especially over the normal acidic pH values found in the vagina. Suitable matrix-forming compounds include, for example, alginic acid, chitosan, gellan gum, poloxamer, and the like. Alginic acid is a generally linear glycouronan polymer containing a mixture of -(1,4)-D-gulosyuronic acid and -(1,4)-D-gulosyuronic acid residues. Generally, the molecular weight of the alginic acid is the range of about 20,000 to about 300,000 g/mole, in other embodiments in the range of about 20,000 to about 250,000 g/mole, and in further embodiments about 240,000 g/mole. Alginic acid is expected to form insoluble alginates by interacting with monovalent and divalent cations (especially Na+, K+, and Ca++) in seminal plasma. Since vaginal fluids generally contain very little Ca++, the semisolid matrix is formed only when ejaculate is present. Alginates also swell in contact with water, thereby assisting in maintaining the desired gel or matrix structure within the vagina.

Bioadhesive compounds suitable for use in composition of use in the present methods include, for example, xanthan gum, hydroxypropyl cellulose, hydroxypropyl methyl cellulose, hydroxyethyl cellulose, sodium carboxymethyl cellulose, chitosan, polycarbophil, carbopol, and the like. The composition can also include lactic acid or other buffering agents that act to maintain the pH of the vagina within its normal acidic range (i.e., a pH of less than about 5 and more preferably in the range of about 3.5 to about 4.5) even in the presence of normal amounts of ejaculate. Besides lactic acid, suitable buffering agents include, but are not limited to, for example, citric acid, potassium acid tartrate, potassium bitartrate, benzoic acid, alginic acid, sorbic acid, fumaric acid, ascorbic acid, stearic acid, oleic acid, tartaric acid, edetic acid ethylenediaminetetracetic acid, acetic acid, and malic acid. The acids may be added as free acids, hydrates, or pharmaceutically acceptable salts. The free acids can be converted to the corresponding salts in the vagina. Buffering agents can also be included.

Additional optional excipients that can be included with the effective amount of the SAMT-247 microbicide include humectants. Suitable humectants include, but are not limited to, for example, glycerol (also referred to as glycerin or glycerine), polyethylene glycols, propylene glycols, sorbitol, triacetin, and the like. In one exemplary embodiment, glycerol is used to prevent the formation of a dry film on the gel when placed within the vagina. Glycerol may also act as a lubricant. Additionally, the compositions may also include a preservative. Suitable preservatives include, but are not limited to, for example, benzoic acid, sodium benzoate, methylparaben, ethylparaben, butylparaben, propylparaben, benzyalkonium chloride, phenylmercuric nitrate, chlorhexidine, and the like.

The effective amount of the SAMT-247 microbicide can be included in a vaginal ring. In one embodiment, the ring is a matrix-type ring. In another embodiment, the ring is a platinum-catalyzed ring. In another embodiment, the ring comprises a silicone polymer, an EVA polymer, or a polyurethane polymer. In another embodiment, the ring is a reservoir-type ring comprising a core and a sheath. In one embodiment, the effective amount of the SAMT-247 microbicide is present in the core of the reservoir-type ring, and the sheath is blank. In one embodiment, the core is platinum-catalyzed. In one embodiment, the core comprises a silicone polymer, an EVA polymer, or a polyurethane polymer. Several single-indication intravaginal rings are currently available, including ESTRING® and FEMRING®, for the treatment of symptoms of post-menopause, and NUVARING®, a contraceptive vaginal ring. Intravaginal rings are disclosed in U.S. Pat. No. 6,951,654, U.S. Patent Application Publication Nos. US2007/0043332 and US2009/0004246, PCT

Publication Nos. WO99/50250, WO 02/076426 and WO 03/094920, the entire contents of each of which are expressly incorporated herein by reference.

The intravaginal rings can provide controlled release of the effective amount the SAMT-247 microbicide and may have any shape and be of any dimensions compatible with intravaginal administration to a female human. Such a ring can be self-inserted into the vagina, where it is held in place due to its shape and inherent elasticity. In one embodiment, the intravaginal ring has an outer diameter of 56 mm. In another embodiment, the intravaginal ring has an outer diameter of about 50 mm, about 51 mm, about 52 mm, about 53 mm, about 54 mm, about 55 mm, about 56 mm, about 57 mm, about 58 mm, about 59 mm or about 60 mm. In another embodiment, the intravaginal ring has a cross-sectional diameter of 7.7 mm. In yet another embodiment, the intravaginal ring has a cross-sectional diameter of about 7.0 mm, about 7.1 mm, about 7.2 mm, about 7.3 mm, about 7.4 mm, about 7.5 mm, about 7.6 mm, about 7.7 mm, about 7.8 mm, about 7.9 mm, about 8.0 mm, about 8.1 mm, about 8.2 mm, about 8.3 mm, about 8.4 mm, or about 8.5 mm

In one embodiment, the intravaginal ring comprises a silicone elastomer. In yet another embodiment, the intravaginal ring comprises a silicone elastomer and a silicone dispersant. In another embodiment, the intravaginal ring comprises a polyurethane thermoplastic polymer or an EVA polymer. The intravaginal ring may include other pharmaceutically compatible agents. Such agents include pharmacologically active agents, as well as, pharmacologically inactive agents known in the art as pharmaceutical excipients. Examples of pharmacologically active agents that may be advantageous include, but are not limited to, a local anesthetic such as lidocaine or a local analgesic or a mixture thereof. Examples of pharmacologically inactive agents that may be advantageous include, but are not limited to, a buffer (or buffers), or hydrophilic compounds that enhance the rate of release of the agent from the device, such as for example, polyvinylpyrrolidone (PVP or povidone), modified cellulose ethers (e.g., hydroxyethylcellulose, hydroxypropylcellulose and hydroxypropylmethylcellulose) microcrystalline cellulose, polyacrylic acid, carbomer, alginic acid, carrageenan, cyclodextrins, dextrin, guar gum, gelatin, xanthan gum and sugars (e.g., monosaccharides such as glucose, fructose and galactose, and dissaccharides such as lactose, maltose and fructose). When employed, the release rate enhancing excipient may be, for example, an amount of about 0.5 to about 40 w/w % and preferably about 2.5 to about 15 w/w % of the device.

The dose administered to a mammal, particularly, a human, in accordance with the present methods should be sufficient to inhibit HIV. One skilled in the art will recognize that dosage will depend upon a variety of factors, including the age, condition, and body weight of the human, as well as the source, particular type of the disease, and extent of the disease in the human. The size of the dose will also be determined by the route, timing and frequency of administration as well as the existence, nature, and extent of any adverse side effects that might accompany the administration of a particular compound and the desired physiological effect. It will be appreciated by one of skill in the art that various conditions or disease states may require prolonged treatment involving multiple administrations.

The therapeutically effective amount of the SAMT-247 microbicide administered can vary depending upon the desired effects and the factors noted above. Typically, dosages will be between 0.01 mg/kg and 250 mg/kg of the subject's body weight, and more typically between about 0.05 mg/kg and 100 mg/kg, such as from about 0.2 to about 80 mg/kg, from about 5 to about 40 mg/kg or from about 10 to about 30 mg/kg of the subject's body weight. Thus, unit dosage forms can be formulated based upon the suitable ranges recited above and the subject's body weight. The term “unit dosage form” as used herein refers to a physically discrete unit of therapeutic agent appropriate for the subject to be treated.

Alternatively, dosages are calculated based on body surface area and from about 1 mg/m² to about 200 mg/m², such as from about 5 mg/m² to about 100 mg/m² will be administered to the subject per day. In particular embodiments, administration of the therapeutically effective amount of the compound involves administering to the subject from about 5 mg/m² to about 50 mg/m², such as from about 10 mg/m² to about 40 mg/m² per day. It is currently believed that a single dosage of the compound is suitable, however a therapeutically effective dosage can be supplied over an extended period of time or in multiple doses per day. Thus, unit dosage forms also can be calculated using a subject's body surface area based on the suitable ranges recited above and the desired dosing schedule. One exemplary formulation is 2 ml of 0.1% SAMT-247 formulated in hydroxyl ethyl cellulose for vaginal administration.

### IV. Methods

Methods are disclosed herein for inhibiting an HIV infection in a subject, such as an HIV-1 infection. The methods include administering to the subject an effective amount of a recombinant gp120 protein comprising a deletion of HIV-1 Envelope (Env) residues 137-152 according to the HXBc2 numbering system (a recombinant HIV-1 gp120 proteins that include a V1 domain deletion), or a nucleic acid molecule encoding the recombinant gp120 protein, wherein the recombinant gp120 protein elicits an immune response to HIV-1; and administering to the subject an effective amount of a SAMT-247 microbicide. The methods can be used to inhibit HIV-1 acquisition in seronegative subject (e.g., by inducing an immune response that protects against HIV-1 infection). In some embodiments the methods involve selecting a subject at risk for contracting HIV-1 infection, and administering to the subject an effective amount of a recombinant gp120 protein comprising a deletion of HIV-1 Envelope (Env) residues 137-152 according to the HXBc2 numbering system, or a nucleic acid molecule encoding the recombinant gp120 protein, wherein the recombinant gp120 protein elicits an immune response to HIV-1; and administering to the subject an effective amount of a SAMT-247 microbicide. The subject can be any subject, including, but not limited to, sex workers or any subject that may have unprotected intercourse. The subject can be male or female.

In some embodiments, the administration of the SAMT-247 microbicide increases zinc availability in a tissue in the subject. In further embodiments, the administration of the SAMT-247 microbicide augments vaccine-induced protective natural killer cell (NK) cytotoxicity and monocyte efferocytosis, and/or decreases T-cell activation in the subject. In further embodiments, administration of the SAMT-247 microbicide increases ADCC and IL17 production in mucosal NKp44 cells. In yet other embodiments, the SAMT-247 microbicide, alone increases IL-10 expression in CD14⁺ monocytes. In some embodiments, one or more of these parameters can be measured in a sample from the subject. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize HIV-1 infection. At risk subjects include sex workers and subjects that may have unprotected intercourse. In accordance with these methods and principles, a composition can be administered according to the teachings herein, or other conventional methods, as an independent prophylaxis or treatment program, or with other agents, such as anti-retroviral agents.

Any of the recombinant gp120 proteins comprising a deletion of HIV-1 Envelope (Env) residues 137-152 according to the HXBc2 numbering system, that elicits an immune response to HIV, as discussed in detail above, can be used in the present methods. The effective amount of the SAMT-247 microbicide can be SAMT-247, a salt or derivative thereof. The effective amount of the SAMT-247 microbicide can be a prodrug form of SAMT-247. The recombinant gp120 protein can be included in a first composition, optionally including an adjuvant, that is administered to the subject, and the SAMT-247 microbicide can be administered in a second composition that is administered to the subject. These compositions can be administered by different routes.

The recombinant HIV-1 gp120 proteins that include a V1 domain deletion can be used in coordinate (or prime-boost) immunization protocols or combinatorial formulations. In certain embodiments, combinatorial immunogenic compositions and coordinate immunization protocols employ separate immunogens or formulations, each directed toward eliciting an anti-HIV-1 immune response, such as an immune response to HIV-1 Env protein. Separate immunogenic compositions that elicit the anti-HIV-1 immune response can be combined in a polyvalent immunogenic composition administered to a subject in a single immunization step, or they can be administered separately (in monovalent immunogenic compositions) in a coordinate immunization protocol.

In one embodiment, a suitable immunization regimen includes at least two separate inoculations with one or more immunogenic compositions including a disclosed immunogen, with a second inoculation being administered more than about two, about three to eight, or about four, weeks following the first inoculation. A third inoculation can be administered several months after the second inoculation, and in specific embodiments, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of HIV-1 infection or progression to AIDS, improvement in disease state (e.g., reduction in viral load), or reduction in transmission frequency to an uninfected partner. If such monitoring indicates that vaccination is sub-optimal, the subject can be boosted with an additional dose of immunogenic composition, and the vaccination parameters can be modified in a fashion expected to potentiate the immune response. Thus, for example, a dose of a disclosed immunogen can be increased or the route of administration can be changed.

It is contemplated that there can be several boosts, and that each boost can be a different immunogen. It is also contemplated in some examples that the boost may be the same immunogen as another boost, or the prime.

The prime and the boost can be administered as a single dose or multiple doses, for example, two doses, three doses, four doses, five doses, six doses or more can be administered to a subject over days, weeks or months. Multiple boosts can also be given, such one to five, or more. Different dosages can be used in a series of sequential inoculations. For example, a relatively large dose in a primary inoculation and then a boost with relatively smaller doses. The immune response against the selected antigenic surface can be elicited by one or more inoculations of a subject.

In several embodiments, a recombinant HIV-1 gp120 protein that includes a V1 domain deletion, or a nucleic acid molecule encoding the recombinant gp120 protein, can be administered to the subject simultaneously with the administration of an adjuvant. In other embodiments, the recombinant HIV-1 gp120 protein that includes a V1 domain deletion can be administered to the subject after the administration of an adjuvant and within a sufficient amount of time to elicit the immune response. The adjuvant can be an aluminum adjuvant. The adjuvant can include monophosphoryl lipid A and/or saponin QS21.

A booster vector encoding HIV-1 envelope (env), glysosaminoglyan (gag), and polymerase (pol) can be administered to the subject. A booster vector encoding HIV-1 envelope (env) and polymerase (pol) can be administered to the subject.

Determination of effective dosages in this context is typically based on animal model studies followed up by human clinical trials and is guided by administration protocols that significantly reduce the occurrence or severity of targeted disease symptoms or conditions in the subject, or that elicit a desired response in the subject (such as a neutralizing immune response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer an effective amount of the composition (for example, amounts that are effective to elicit a desired immune response or alleviate one or more symptoms of a targeted disease). In alternative embodiments, an effective amount or effective dose of the composition may simply inhibit or enhance one or more selected biological activities correlated with a disease or condition, as set forth herein, for either therapeutic or diagnostic purposes.

Dosage of a composition can be varied by the attending clinician to maintain a desired concentration at a target site (for example, systemic circulation). Higher or lower concentrations can be selected based on the mode of delivery, for example, trans-epidermal, rectal, oral, pulmonary, or intranasal delivery versus intravenous or subcutaneous delivery. The actual dosage of the recombinant HIV-1 gp120 protein that includes a V1 domain deletion will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response. Similarly, the dosage of the SAMT-247 microbicide can be varied based on the route of administration, and clinical parameters of the subject (for example, the subject's age, size, fitness, extent of symptoms, susceptibility factors, and the like).

A non-limiting range for an effective amount of the recombinant HIV-1 gp120 protein that includes a V1 domain deletion within the methods and immunogenic compositions of the disclosure is about 0.0001 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example, 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. In some embodiments, the dosage includes a set amount of a recombinant HIV-1 gp120 protein that includes a V1 domain deletion such as from about 1-300 μg, for example, a dosage of about 1, 2, 3, 4, 5, 6, 7, 8, 9, 10, 15, 20, 30, 40, 50, 60, 70, 80, 90, 100, 150, 200, 250, or about 300 μg.

The dosage and number of doses will depend on the setting, for example, in an adult or anyone primed by prior HIV-1 infection or immunization, a single dose may be a sufficient booster. In naïve subjects, in some examples, at least two doses would be given, for example, at least three doses. In some embodiments, an annual boost is given, for example, along with an annual influenza vaccination.

HIV-1 infection does not need to be completely inhibited for the methods to be effective. For example, elicitation of an immune response to HIV-1 with one or more of the disclosed immunogens can reduce or inhibit HIV-1 infection by a desired amount, for example, by at least 10%, at least 20%, at least 50%, at least 60%, at least 70%, at least 80%, at least 90%, at least 95%, at least 98%, or even at least 100% (elimination or prevention of detectable HIV-1 infected cells), as compared to HIV-1 infection in the absence of the therapeutic agent. In additional examples, HIV-1 replication can be reduced or inhibited by the disclosed methods. HIV-1 replication does not need to be completely eliminated for the method to be effective. For example, the immune response elicited using one or more of the disclosed immunogens can reduce HIV-1 replication by a desired amount, for example, by at least 10%, at least 20%, at least 50%, at least 60%, at least 70%, at least 80%, at least 90%, at least 95%, at least 98%, or even at least 100% (elimination or prevention of detectable HIV-1 replication), as compared to HIV-1 replication in the absence of the immune response.

To successfully reproduce itself, HIV-1 must convert its RNA genome to DNA, which is then imported into the host cell's nucleus and inserted into the host genome through the action of HIV-1 integrase. Because HIV-1′s primary cellular target, CD4+ T-Cells, can function as the memory cells of the immune system, integrated HIV-1 can remain dormant for the duration of these cells' lifetime. Memory T-Cells may survive for many years and possibly for decades. This latent HIV-1 reservoir can be measured by co-culturing CD4+ T-Cells from infected patients with CD4+ T-Cells from uninfected donors and measuring HIV-1 protein or RNA (See, e.g., Archin et al., AIDS, 22:1131-1135, 2008). In some embodiments, the provided methods of treating or inhibiting HIV-1 infection include reduction or elimination of the latent reservoir of HIV-1 infected cells in a subject. For example, a reduction of at least 10%, at least 20%, at least 50%, at least 60%, at least 70%, at least 80%, at least 90%, at least 95%, at least 98%, or even at least 100% (elimination of detectable HIV-1) of the latent reservoir of HIV-1 infected cells in a subject, as compared to the latent reservoir of HIV-1 infected cells in a subject in the absence of the treatment with one or more of the provided immunogens.

Following immunization of a subject, serum can be collected from the subject at appropriate time points, frozen, and stored for neutralization testing. Methods to assay for neutralization activity, include, but are not limited to, plaque reduction neutralization (PRNT) assays, microneutralization assays, flow cytometry based assays, single-cycle infection assays (e.g., as described in Martin et al. (2003) Nature Biotechnology 21:71-76), and pseudovirus neutralization assays (e.g., as described in Georgiev et al. (Science, 340, 751-756, 2013), Seaman et al. (J. Virol., 84, 1439-1452, 2005), and Mascola et al. (J. Virol., 79, 10103-10107, 2005), each of which is incorporated by reference herein in its entirety. In some embodiments, the serum neutralization activity can be assayed using a panel of HIV-1 pseudoviruses as described in Georgiev et al., Science, 340, 751-756, 2013 or Seaman et al. J. Virol., 84, 1439-1452, 2005. Briefly, pseudovirus stocks are prepared by co-transfection of 293T cells with an HIV-1 Env-deficient backbone and an expression plasmid encoding the Env gene of interest. The serum to be assayed is diluted in Dulbecco's modified Eagle medium—10% FCS (Gibco) and mixed with pseudovirus. After 30 min, 10,000 TZM-bl cells are added, and the plates are incubated for 48 hours. Assays are developed with a luciferase assay system (Promega, Madison, WI), and the relative light units (RLU) are read on a luminometer (Perkin-Elmer, Waltham, MA). To account for background, a cutoff of ID₅₀≥40 can be used as a criterion for the presence of serum neutralization activity against a given pseudovirus.

In some embodiments, administration of an effective amount of one or more of recombinant HIV-1 gp120 proteins that includes a V1 domain deletion to a subject (e.g., by a prime-boost administration of a DNA or RNA vector encoding a recombinant HIV-1 gp120 protein that includes a V1 domain deletion followed by a protein boost) elicits a neutralizing immune response in the subject, wherein serum from the subject neutralizes, with an ID₅₀≥40, at least 10% (such as at least 15%, at least 20%, at least 30%, at least 40%, at least 50%, or at least 70%) of pseudoviruses is a panel of pseudoviruses including the HIV-1 Env proteins listed in Table S5 or Table S6 of Georgiev et al. (Science, 340, 751-756, 2013), or Table 1 of Seaman et al. (J. Virol., 84, 1439-1452, 2005). As noted above, an adjuvant can also be administered, such as an aluminum adjuvant to an adjuvant including monophosphoryl lipid A and/or saponin QS21.

One approach to administration of nucleic acids is direct immunization with plasmid DNA, such as with a mammalian expression plasmid. Immunization by nucleic acid constructs is taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In some embodiments, a plasmid DNA vaccine is used to express a recombinant HIV-1 gp120 protein that includes a V1 domain deletion in a subject. For example, a nucleic acid molecule encoding a recombinant HIV-1 gp120 protein that includes a V1 domain deletion can be administered to a subject to elicit an immune response to HIV-1 gp120. In some embodiments, the nucleic acid molecule can be included on a plasmid vector for DNA immunization, such as the pVRC8400 vector (described in Barouch et al., J. Virol, 79, 8828-8834, 2005, which is incorporated by reference herein).

RNA based vaccines are of use in the disclosed methods. In another approach to using nucleic acids for immunization, an immunogen (such as a protomer of a HIV-1 Env ectodomain trimer) can be expressed by attenuated viral hosts or vectors or bacterial vectors. Recombinant vaccinia virus, adeno-associated virus (AAV), herpes virus, retrovirus, cytomegalovirus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one embodiment, a nucleic acid encoding a recombinant HIV-1 gp120 protein that includes a V1 domain deletion is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

In some embodiments, an immunization protocol that mirrors the “RV144” trial is used with the immunogens provided herein. As discussed in Rerks-Ngarm et al. (New Eng J Med. 361 (23): 2209-2220, 2009, incorporated by reference herein) RV144 was a phase III trial of a prime-boost HIV-1 vaccine consisting of four injections of ALVAC HIV (vCP1521) followed by two injections of AIDSVAX B/E. ALVAC HIV (vCP1521) is a canarypox vector containing HIV-1 env, gag, and pol genes, and AIDSVAX B/E is a genetically engineered form of gp120. The env gene of ALVAC HIV (vCP1521) and the AIDSVAX B/E gp120 can be modified to encode or contain the V1 deletion provided herein (deletion of residues 137-152 according to HXBc2 numbering) and administered to a subject using the RV144 prime-boost protocol (or any other suitable protocol). An exemplary protocol includes, but is not limited to, a) administering to the subject an effective amount of a composition comprising a prime immunization of a DNA vector encoding HIV-1 Env with a deletion of HIV-1 Env residues 137-152 according to the HXBc2 numbering system and an adjuvant, b) administering to the subject a boost immunization of a vector encoding HIV env, HIV gag, and HIV pol and an alum adjuvant, c) administering to the subject a boost immunization of a purified gp120 protein with a deletion of HIV-1 Env residues 137-152 according to the HXBc2 numbering system formulated with an effective amount of an alum adjuvant; and d) applying intra-vaginally an effective amount of a SAMT-247 microbicide, thereby inhibiting HIV-1 acquisition by the subject. In some embodiments, the SAMT-247 microbicide can be administered intravaginally prior to the HIV-1 exposure. In other embodiments, the SAMT-247 microbicide is administered within 4 hours of an HIV-1 exposure, such as within about 5, 10, 15, or 30 minutes, or within about 1, 2, 3 or 4 hours of an HIV exposure. In an embodiment, the SAMT-247 microbicide is administered within 3 hours of an HIV exposure. The exposure can be from sexual intercourse.

In some embodiments, a modified form of the “RV144” immunization protocol can be used. For example, there can be additional (or fewer) prime or boost administrations, and the initial prime can be a DNA based immunization including a plasmid vector encoding HIV-1 Env with or without the V1 deletion as disclosed herein.

The administration of the recombinant gp120 protein comprising a deletion of HIV-1 Envelope (Env) residues 137-152 according to the HXBc2 numbering system can be preformed at any time prior to the exposure, including but not limited to, at least about 2, 3, 4, 5, 6, 7, 8, 9, 10, 11 or 12 weeks prior to the exposure. However, this administration can be performed at about 1, 2, 3, 4, 5, 6, 7, 8, 9, 10, 11 or 12 months prior to the exposure, or at about 1, 2, 3, 4, 5, 6, 7, 8, 9, or 10 years prior to the exposure, as long as the immune response is induced upon the subsequent exposure to HIV. In some embodiments, the administration is performed about 2 weeks to about 10 years prior to the exposure, such as about 2 months to about 5 years prior to the exposure, or within 1 to 5 years of the exposure.

In some embodiments, the subject is a female, and the SAMT-247 microbicide can be administered intravaginally prior to the HIV-1 exposure. In other embodiments, the SAMT-247 microbicide is administered within 4 hours of an HIV-1 exposure, such as within about 5, 10, 15, or 30 minutes, or within about 1, 2, 3 or 4 hours of an HIV exposure. In an embodiment, the SAMT-247 microbicide is administered within 4 hours of an HIV exposure. In an embodiment, the SAMT-247 microbicide is administered within 3 hours of an HIV exposure. The SAMT-247 microbicide can be administered by the vaginal route using a suppository, cream, or a gel. The SAMT-247 microbicide can be administered by the vaginal route using a vaginal ring delivery device.

In some embodiments, the subject is a male or female, and the SAMT-247 microbicide can be administered intrarectally prior to the HIV-1 exposure. In other embodiments, the SAMT-247 microbicide is administered within 4 hours of an HIV-1 exposure, such as within about 5, 10, 15, or 30 minutes, or within about 1, 2, 3 or 4 hours of an HIV exposure. In an embodiment, the SAMT-247 microbicide is administered within 4 hours of an HIV exposure. In another embodiment, the SAMT-247 microbicide is administered within 3 hours of an HIV exposure.

Without limitation, the SAMT-247 microbicide can be administered by the rectal route using a suppository, cream, or a gel. The SAMT-247 microbicide can be administered as a rectal lavage. The SAMT-247 microbicide can be administered as a rectal suppository. SAMT-247 microbicide can be administered using a condom or in the form of a lubricant. The SAMT-247 microbicide can be administered by the vaginal route using a suppository, cream, gel, or intravaginal ring. The SAMT-247 microbicide can be administered as a vaginal lavage. The SAMT-247 microbicide can be administered as a vaginal suppository. SAMT-247 microbicide can be administered in form of a vaginal lubricant.

In some embodiments, the administration of the SAMT-247 microbicide increases zinc availability in a tissue in the subject. In further embodiments, the administration of the SAMT-247 microbicide augments vaccine-induced protective natural killer cell (NK) cytotoxicity and monocyte efferocytosis, and/or decreases T-cell activation in the subject. In further embodiments, administration of the SAMT-247 microbicide increases ADCC and IL17 production in mucosal NKp44 cells. In yet other embodiments, the SAMT-247 microbicide increases IL-10 expression in CD14⁺ monocytes. In some embodiments, one or more of these parameters can be measured in a sample from the subject. Without being bound by theory, one or more of these effects augment the effect of administration of the effective amount of a recombinant gp120 protein comprising a deletion of HIV-1 Envelope (Env) residues 137-152 according to the HXBc2 numbering system (a recombinant HIV-1 gp120 proteins that include a V1 domain deletion), or a nucleic acid molecule encoding the recombinant gp120 protein, wherein the recombinant gp120 protein elicits an immune response to HIV-1.

In yet other embodiments, the methods can include administering one or more of other drugs for treating HIV, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers. In other embodiments, these agents are not administered to the subject.

### EXAMPLES

The HIV epidemic continues unabated in sub-Saharan Africa and particularly impacts adolescent women with limited access to antiretroviral therapy. It is disclosed herein that the risk of vaginal SIV_{mac251} acquisition is reduced more than 90% by combining vaccination with V1-deleted (V2 enhanced) SIV envelope immunogens and topical treatment with the zinc-finger inhibitor SAMT-247. 80% of macaques vaccinated and treated with SAMT-247 remained uninfected following 14 weekly exposures to the highly pathogenic SIV_{mac251}. The combined SAMT-247/vaccine regimen was significantly more efficacious than vaccination alone. By increasing zinc availability, SAMT-247 augments vaccine-induced protective NK cytotoxicity and monocyte efferocytosis, decreases T-cell activation, and dramatically augments the effect of vaccination.

### Example 1

### Materials and Methods

Animals. Fifty female Indian Rhesus macaques obtained from the free-range breeding colony on Morgan Island, South Carolina, were used in this study. The macaques, aged 2 to 3 years at study initiation, were negative for SIV, simian retrovirus, and STLV, and were MHC typed before being divided into 4 groups: vaccine/microbicide group (20 macaques, 3 A*01 positive, 1 B*17 positive, 1 B*08 positive); vaccine only group (18 macaques, 3 A*01 positive, 1 B*17 positive); microbicide only group (6 macaques, 1 both A*01 positive and B*17 positive) and control group (6 macaques, 1 both A*01 positive and B*17 positive). Data on 37 historical naïve controls intravaginally challenged with the same dose and lot of SIV_{mac251} were added to the control group to increase statistical power. There was no difference between the concurrent and historical controls in rate of SIV acquisition (p=0.66).

Animals were closely monitored daily for any signs of illness, and appropriate medical care was provided as needed. Animals were socially housed per the approved ACUC protocol and social compatibility except during the viral challenge phase when they were individually housed. All clinical procedures, including biopsy collection, administration of anesthetics and analgesics, and euthanasia, were carried out under the direction of a laboratory animal veterinarian. Steps were taken to ensure the welfare of the animals and minimize discomfort of all animals used in this study. Animals were fed daily with a fresh diet of primate biscuits, fruit, peanuts, and other food items to maintain body weight or normal growth. Animals were monitored for psychological well-being and provided with physical enrichment including sanitized toys, destructible enrichment (cardboard and other paper products), and audio and visual stimulation.

Additionally, rectal tissue from 9 macaques was used for an in vitro study. The age-matched macaques from this separate study (P204) included 4 animals vaccinated against HIV and 5 naïve animals, all challenged with SHIV. Rectal tissue was obtained post challenge from 7 infected and 2 uninfected animals for the P204 study analysis. Extra cells were available from that collection and were used here for the in vitro study.

Human Healthy Volunteers. Blood from eight healthy human volunteers was obtained from a blood bank.

Immunization and Challenge. Macaques in the vaccine/microbicide and vaccine only groups were immunized at weeks 0 and 4 with DNA encoding SIVgp160ΔV1 (2 mg/dose) and SIV239gag (1 mg/dose) in a total volume of 1 ml PBS. The DNA was administered in both thighs (0.5 ml to each). At 8 weeks the macaques were administered ALVAC encoding gag/pro/env (wildtype env) in the right thigh, 10⁸ pfu/dose in 1 ml PBS. At week 12 the macaques were boosted with the same ALVAC and in addition SIVgp120ΔV1 protein (400 μg/dose in 1 ml PBS plus 1 ml 2% alhydrogel). The ALVAC was administered to the right thigh; the 2 ml dose of Env protein plus alum was administered to the left thigh. Beginning at week 17 all macaques were challenged intravaginally weekly with 1 ml of a SIV_{mac251} stock containing 800 tissue culture infectious doses. Up to 14 challenges were administered until the macaques became SIV positive as assessed by droplet digital PCR. Two ml doses of the microbicide, SAMT-247, were administered as a gel intravaginally to macaques in the vaccine/microbicide group and the microbicide only group 4 hours prior to each SIV challenge. The 2 ml gel contained 0.8% SAMT-247 in hydroxyethyl cellulose (HEC) gel (2.7% Natrosol cellulose 250HX Pharma, 0.01% DMSO, and 0.9% saline). Macaques in the vaccine only group and control group received HEC gel only lacking the SAMT-247.

IgG plasma titers to gp120. gp120 total IgG antibodies were measured by ELISA. ELISA plates (Nunc Maxisorp 96 well plate) were coated with 100 μl of 500 ng/ml SIV_{mac251-M766} gp120 protein/well in 50 mM sodium bicarbonate buffer pH 9.6 and incubated overnight at 4° C. Plates were blocked with 200 μl PBS SUPERBLOCK™ (Thermo Fisher Scientific) for 1 h at RT. Plasma samples were serial-diluted with sample diluent (Avioq), and 100 μl of diluted plasma was added to the wells. Plates were covered and incubated for 1 h at 37° C., washed 6 times with PBS Tween 20 (0.05%), and incubated with 100 μl anti-human HRP diluted at 1:120,000 in sample diluent (Avioq) for 1 h covered at 37° C. The plates were washed 6 times. Plates were developed using 100 μl K-Blue Aqueous substrate (Neogen) to all wells and incubated for 30 min at RT. The reaction was stopped by the addition of 100 μl 2N Sulfuric acid to all wells and the plate was read at 450 nm on a Molecular Devices E-max plate reader.

Pepscan. Plasma samples were assayed by PEPSCAN analysis using SIV_{mac251} gp120 linear peptides as previously described (Silva de Castro, I., et al., iScience 24, 102047 (2021)). ELISA plates (Nunc Maxisorp) were coated with 100 ng of each of the 1 to 89 overlapping peptides (with 15 amino acids each encompassing the entire SIV_{mac251} gp120 sequence) in 50 mM NaHCO3, pH 9.6, per well, incubated overnight at 4° C., and blocked with 200 μl of Pierce SUPERBLOCK™ blocking buffer in PBS for 1 hour at room temperature (RT). Serum samples were diluted at 1:50 in sample diluent (Avioq), and 100 μl were added to the plate and incubated for 1 h at 37° C. Plates were washed 6 times with PBS TWEEN® 20 (0.05%) and incubated with 100 μl anti-human HRP diluted at 1:120,000 in sample diluent (Avioq) to all wells and incubated, covered, for 1 hour at 37° C. The plates were again washed 6 times and developed using 100 μl K-Blue Aqueous substrate (Neogen) to all wells and incubated 30 min at RT. The reaction was stopped by adding 100 μl 2N Sulfuric acid to all wells and read plate at 450 nm on a Molecular Devices E-max plate reader.

ADCC CEM-based assay. ADCC activity was assessed as previously described using EGFP-CEM-NKr-CCR5-SNAP cells that constitutively express GFP as targets (Rahman, M. A., et al, J Immunol 203, 2459-2471(2019)). Briefly, one million target cells were incubated with 50 μg of ΔV1 gp120 protein for 2 h at 37° C. After this coating, the target cells were washed and labeled with SNAP-SURFACE® ALEXA FLUOR® 647 (New England Biolabs, Connecticut, USA S9136S) per manufacturer recommendations for 30 min at RT. Plasma samples, heat inactivated at 56° C. for 30 min, were serially diluted (7 ten-fold dilutions starting at 1:10) and 100 μl were added to wells of a 96-well V-bottom plate (Millipore Sigma). 5000 target cells (50 μl) and 250,000 human PBMCs (50 μl) were added as effectors to each well to give an effector/target (E/T) ratio of 50:1. The plate was incubated at 37° C. for 2 h followed by two PBS washes. The cells were resuspended in 200 μl of a 2% PBS-paraformaldehyde solution and acquired on a Symphony equipped with a high throughput system (BD Biosciences, San Jose, California, USA). Specific killing was measured by loss of GFP from the SNAPAlexa647+ target cells. Target and effector cells cultured in the presence of R10 medium were used as background. Anti-SIVmac gp120 monoclonal antibody KK17 (from the NIH AIDS reagent program) was used as a positive control. Normalized percent killing was calculated as: (killing in the presence of plasma-background)/(killing in the presence of KK17-background) X100. The ADCC endpoint titer is defined as the reciprocal dilution at which the percent ADCC killing was greater than the mean percent killing of the background wells containing medium only with target and effector cells, plus three standard deviations.

Inhibition of ADCC CEM-based assay by monoclonal F(ab′)2 of NCI05 and NCI09. F(ab′)2 fragments were prepared from both NCI05 or NCI09 (as these antibodies recognize overlapping conformationally distinct V2 epitopes (Silva de Castro, I., et al., iScience 24, 102047 (2021)) mAb using Pierce f(ab′)2 Micro Preparation Kit (Cat #44688, Thermo scientific) following the manufacturer's instructions. An SDS-page gel with the recovered F(ab′)2 was run and Silver stained (Cat #LC6070, Silver Quest staining Kit, Invitrogen) according to the manufacturer's instructions, to assure the purity of the F(ab′)2 fragments. Target cells, coated with ΔV1 gp120 protein as indicated above and labeled with SNAP-SURFACE® ALEXA FLUOR® 647, were incubated for 1 h at 37° C. with 5 μg/ml of purified F(ab′)2 fragments from NCI05, or NCI09 monoclonal antibodies. Cells incubated without F(ab′)2 served as control. These target cells were subsequently used in the ADCC assay as described above.

ADCC assay using SAMT-247. Ten million human PBMC effector cells were resuspended in 1 ml R10 medium and incubated in the presence/absence of 100 nM SAMT-247 for 4 hours. Cells were washed and used as effector cells to measure ADCC activity as described above. SAMT-247 induced ADCC was measured by subtracting ADCC with no SAMT-247 treated effector cells from ADCC with SAMT-247 treated effector cells.

Efferocytosis assay. The frequency of efferocytotic CD14⁺ cells was assessed by Efferocytosis Assay kit (#601770, Cayman Chemical company, Ann Arbor, MI, USA). CD14⁺ cells were used as effector cells, whereas apoptotic neutrophils were used as target cells. The protocol was readapted in order to use CD14⁺ monocyte cells rather than differentiated macrophages due to the low cell availability. CD14⁺ cells were isolated from cryopreserved PBMCs (10×10⁶ cells) collected following pre-study and 2 weeks post last immunization (week 14) or 5 weeks post last immunization (week 17) by using non-human primate CD14 microbeads (#130-091-097, Miltenyi Biotec Inc.) and following manufacturer instructions. At the end of the separation cells were counted and stained with CYTOTELL™ Blue provided by the kit and following manufacturer instructions. One unrelated macaque was used as source of neutrophils as target cells. Neutrophils were isolated as previously described (Khandpur, R., et al., Sci Transl Med 5, 178ra140 (2013)). Briefly, following isolation of PBMCs by Ficoll Plaque (GE Healthcare), the cellular pellet was added to an equal volume of 20% dextran in water, gently mixed, and incubated for 1 min. Approximately three volumes of PBS were added, mixed again and incubated in the dark for 50-60 minutes. At the end of incubation, the clear layer at the top of the tube containing neutrophils was collected. Cells were pelleted and treated with ACK lysing buffer (Quality Biological, Gaithersburg, MD, USA) for 5 min at 37° C., washed with R10 and counted. Neutrophils were stained with CFSE provided by the kit and following manufacturer instructions. The apoptosis of neutrophils was induced by treatment with Staurosporine Apoptosis inducer provided by the kit. Briefly, isolated cells were resuspended in R10 containing Staurosporine diluted 1:1000 and incubated at 37° C. for 3 hours. At the end of the incubation cells were washed twice with R10 and used for the efferocytosis assay. Subsequently, effector and apoptotic target cells were cultured alone (as controls) or cocultured at a ratio of one effector CD14⁺ cell and three target apoptotic neutrophils. Cells were incubated at 37° C. for 12 hours. At the end of the coculture, cells were washed with PBS, fixed with 1% paraformaldehyde in PBS and acquired on a FACSYMPHONY™ A5 and examined using FACSDIVA™ software (BD Biosciences) by acquiring all stained cells. Data were further analyzed using FlowJo v10.1 (Treestar, Inc., Ashland, OR). The frequency of efferocytotic CD14⁺ cells was determined as the frequency of double-positive cells for CYTOTELL™ Blue and CFSE on the CYTOTELL™ Blue positive monocytes.

Efferocytosis assay using SAMT-247 and gp120 pooled peptides. Efferocytosis was done as described above with the exception that SAMT-247 and/or gp120 pooled peptides were added to the co-culture for 12 hours. SAMT-247 induced Efferocytosis was measured by subtracting Efferocytosis with no SAMT-247 treated effector cells from Efferocytosis with SAMT-247 treated effector cells.

Rectal mucosal NK/ILC phenotyping and cytokine expression upon gp120 peptides/PMA stimulation. The frequency and cytokine levels of NK/ILCs were measured in macaque rectal mucosa pre vaccination and 1 week post last vaccination (week 13). Freshly collected rectal biopsies were processed to the single cell level. A portion of the cells were phenotyped and the rest were cultured in R10 in the presence/or absence of gp120 peptides or PMA for 12 hours. Subsequently, cells were stained with Live/Dead Aqua Dye (cat. #L34966, 0.5 μl) from Thermofisher, followed by surface staining with the following: Alexa 700 anti-CD3 (SP34-2; cat. #557917, 5 μl), Alexa 700 anti-CD20 (2H7; cat. #560631, 5 μl), Alexa 700 anti-CD11b (ICRF44; cat. #557918, 5 μl), APC-Cy7 anti-CD16 (3G8; cat. #557758, 5 μl), PE-CF594 anti-CD56 (B159; cat. #562289, 5 μl) BV650 anti-NKp44 (P44-8; cat. #744302, 5 μl), BV786 anti-CD45 (D058-1283; cat. #563861, 5 μl) from BD Biosciences; and PE-Cy7 anti-NKG2A (Z199; cat. #B10246, 5 μl) from Beckman Coulter for 30 minutes at room temperature. This was followed by permeabilization with a FOX3-transcription buffer set (cat. #00-5523-00) from eBioscience according to the manufacture's recommendation and subsequently intracellular staining with the following: BV421 anti-IFN-γ (B27; cat. #562988, 5 μl) from BD Biosciences and PE-Cy5.5 anti-IL-17 (BL168; cat. #512314, 5 μl) from Biolegend for 30 minutes at room temperature. Flow cytometry acquisitions were performed on a LSRII and examined using FACSDIVA™ software (BD Biosciences).

Intracellular cytokines of Human blood NK cells using SAMT-247 and PMA stimulation with or without zinc chelator. The levels of NKG2A⁺ NK cells were measured in blood of healthy humans. Human PBMCs were thawed and cultured in R10 in the presence or absence of SAMT-247 and/or PMA for 12 hours. Cells were incubated with or without Zinc chelator TPEN (cat. #P4413-100 MG, 5 μM) for 12 h. Subsequently, PBMCs were surface stained with the following: BUV737 anti-CD3 (SP34-2; cat. #741872, 5 μl), Alexa700 anti-CD20 (2H7; cat. #560631, 5 μl), BV786 anti-CD45 (HI30; cat. #563716, 5 μl) from BD Biosciences; APC-H7 anti-CD11b (ICRF44; cat. #47-0118-42, 5 μl) from eBioscience and PE-Cy7 anti-NKG2A (Z199; cat. #B10246, 5 μl) from Beckman coulter for 30 minutes at room temperature. This was followed by permeabilization with FOX3-transcription buffer set (cat. #00-5523-00) from eBioscience according to the manufacture's recommendation and subsequently intracellular staining with the following: BV750 anti-TNF-α (MAB11; cat. #566359, 5 μl), BUV396 anti-IFN-γ (B27; cat. #563563, 5 μl), BV510 anti-GranB (GB11; cat. #563388, 5 μl) from BD Biosciences; and FITC anti-Perforin (pf-344; cat. #3465-7, 5 μl) from MABTECH for 30 minutes at room temperature. Flow cytometry acquisitions were performed on a FACSymphony A5 and examined using FACSDIVA™ software (BD Biosciences).

Frequencies and cytokine levels of macaque rectal mucosal NK cells and T cells following SAMT-247 and PMA stimulation. NK/ILC and T cell frequencies and cytokine levels were measured in macaque rectal mucosa. Freshly collected rectal biopsies were processed to single cells and cultured in R10 in the presence/or absence of SAMT-247 and/or PMA for 12 hours. Subsequently, cells were stained for live cells with Live/Dead Blue dye (cat. #L34962, 0.5 μl) from Thermofisher; followed by surface staining with the following: BUV737 anti-CD3 (SP34-2; cat. #741872, 5 μl), BV711 anti-CD4 (L200; cat. #563913, 5 μl), BV650 anti-NKp44 (P44-8; cat. #744302, 5 μl), Alexa700 anti-CD20 (2H7; cat. #560631, 5 μl), BV786 anti-CD45 (D058-1283; cat. #563861, 5 μl) from BD Biosciences; APC-H7 anti-CD11b (ICRF44; cat. #47-0118-42, 5 μl), PE-Cy5 anti-CD95 (ICRF44; cat. #15-0959-42, 5 μl) from eBioscience; BV570 anti-CD8 (RPA-T8; cat. #301038, 5 μl), BV605 anti-CCR6 (G034E3; cat. #353420, 5 μl), APC anti-CXCR3 (G025H7; cat. #353708, 5 μl), from Biolegend and PE-Cy7 anti-NKG2A (Z199; cat. #B10246, 5 μl) from Beckman coulter for 30 minutes at room temperature. This was followed by permeabilization with a FOX3-transcription buffer set (cat. #00-5523-00) from eBioscience according to the manufacture's recommendation and subsequently intracellular staining with the following: BV750 anti-TNF-α (MAB11; cat. #566359, 5 μl), BUV395 anti-IFN-γ (B27; cat. #563563, 5 μl), BV510 anti-GranB (GB11; cat. #563388, 5 μl), BV421 anti-IL-10 (JES3-9D7; cat. #564053, 5 μl) from BD Biosciences; PE-Cy5.5 anti-IL-17 (BL168; cat. #512314, 5 μl) from Biolegend; and FITC anti-Perforin (pf-344; cat. #3465-7, 5 μl) from MABTECH for 30 minutes at room temperature. Flow cytometry acquisitions were performed on a FACSymphony A5 and examined using FACSDiva software (BD Biosciences).

Zinc chelation: The frequencies and cytokine levels of NK cells, monocytes and T cells were measured in macaque blood. Cryopreserved PBMCs were thawed and cultured in R10 in the presence/or absence of SAMT-247 and/or gp120 peptides for 12 hours. Cells were incubated with or without Zinc chelator TPEN (cat. #P4413-100 MG, 5 μM) for 12 h. Subsequently, cells were stained for live cells with Live/Dead Blue dye (cat. #L34962, 0.5 μl) from Thermofisher; followed by surface staining with the following: Alexa 700 anti-CD3 (SP34-2; cat. #557917, 5 μl), BV711 anti-CD4 (L200; cat. #563913, 5 μl), BB700 anti-CD8 (RPA-T8; cat. #566452, 5 μl), BUV737 anti-CD20 (2H7; cat. #612848, 5 μl), PE anti-CD45 (D058-1283; cat. #552833, 5 μl), BV786 anti-CCR5 (3A9; cat. #565001, 5 μl), BV496 anti-CD16 (3G8; cat. #612944, 5 μl), BUV661 anti-HLA-DR (G46-6; cat. #612980, 5 μl), BUV805 anti-CD14 (M5E2; cat. #565779, 5 μl) from BD Biosciences; APC-H7 anti-CD11b (ICRF44; cat. #47-0118-42, 5 μl), PE-Cy5 anti-CD95 (ICRF44; cat. #15-0959-42, 5 μl) from eBioscience; APC anti-α₄β₇, provided by the NIH Nonhuman Primate Reagent Resource (R24 OD010976, and NIAID contract HHSN272201300031C); BV605 anti-CCR6 (G034E3; cat. #353420, 5 μl), BV650 anti-CXCR3 (G025H7; cat. #353730, 5 μl), from Biolegend and PE-Cy7 anti-NKG2A (Z199; cat. #B10246, 5 μl) from Beckman coulter for 30 minutes at room temperature. This was followed by permeabilization with a FOX3-transcription buffer set (cat. #00-5523-00) from eBioscience according to the manufacture's recommendation and subsequently intracellular staining with the following: BV750 anti-TNF-α (MAB11; cat. #566359, 5 μl), BUV395 anti-IFN-γ (B27; cat. #563563, 5 μl), BV510 anti-GranB (GB11; cat. #563388, 5 μl), BV421 anti-IL-10 (JES3-9D7; cat. #564053, 5 μl), PE-CF594 anti-Ki67 (B56; cat. #567120, 5 μl) from BD Biosciences and FITC anti-Perforin (pf-344; cat. #3465-7, 5 μl) from MABTECH for 30 minutes at room temperature. Flow cytometry acquisitions were performed on a FACSYMPHONY™ A5 and examined using FACSDIVA™ software (BD Biosciences).

CD4+T cell phenotypes. The levels of CD4+ T cell subsets were measured in blood at baseline and week 13 in vaccinated animals. PBMCs were stained with the following: LIVE/DEAD™ Fixable Blue Dead Cell Stain (cat #L23105 Thermo Fisher); Alexa 700 anti-CD3 (SP34-2; cat. #557917), BV785 anti-CD4 (L200; cat. #563914), PeCy5 anti-CD95 (DX2; cat. #559773), Qdot655 anti-CCR5 (3A9; cat. #564999), BUV496 anti-CD8 (RPA-T8; cat. #564804), BUV737 anti-CD28 (CD28.2; cat. #612815), BUV396 anti-ICOS (C398.4A; cat. #565884) and FITC anti-Ki67 (B56; cat. #556026) from BD Biosciences; APC Cy7 anti-CXCR3 (G025H7; cat. #353722), Qdot605 anti-CCR6 (G034E3; cat. #353420), BV510 anti-CD127 (A019D5; cat. #351332), BV750 anti-PD-1 (EH12.2H7; cat. #329965) and BV711 anti-CD25 (BC96; cat. #302636) from BioLegend (San Diego, California, USA); PE-eFluor 610 anti-CXCR5 (MUSUBEE; cat. #61-9185-42), PE anti-RoRg (AFKJS-9; cat. #12-6988-80), PeCy7 anti-T-bet (4B10; cat. #25-5825-82), Percp eFlour 710 anti-GATA-3 (TWAJ; cat. #46-9966-42), eFluor 450 anti-FoxP3 (236A/E7; cat. #48-4777-42) eBioscience (San Diego, California, USA); and APC anti-α₄β₇, provided by the NIH Nonhuman Primate Reagent Resource (R24 OD010976, and NIAID contract HHSN272201300031C). Samples were acquired on a BD FACSYMPHONY® A5 cytometer and analyzed with FlowJo software 10.6. Gating was done on live CD3+CD4+ cells and on vaccine induced Ki67+ cells. CXCR3 and CCR6 expression was used to identify Th1 or Th2 populations, as previously described (Vaccari, M., et al. Nat Med 24, 847-856 (2018)).

Zinc intensity. NKG2A⁺ NK cells were isolated from cryopreserved healthy human PBMCs. NK cells were labeled with APC anti-NKG2A (Z199, cat. #A60797) from Beckman coulter, followed by separation with APC MicroBeads (#130-090-855, Miltenyi Biotec Inc.) using manufacturer instructions. Subsequently NK cells were cultured in the presence or absence of SAMT-247 and/or PMA stimulation for 7 hours. Cells were plated on ibidi chamber slides. Cells were washed and treated with zinc chelator or remained untreated for 30 minutes according to the manufacturers' recommendation using a cell-based Zinc assay kit (cat. #ab241014, Abcam). Subsequently cells were washed and stained with Zinc probe-green (cat. #ab241014, Abcam) and DAPI (Molecular Probes, Waltham, MA, USA) was used to visualize nuclei. Signals were visualized with a confocal laser-scanning microscope (Leica SP8, Buffalo Grove, IL, USA). Image processing was performed using the Imaris 9.2.1 software.

Statistical analysis. Statistical analysis was performed using the Wilcoxon signed-rank test or Mann-Whitney test to compare continuous factors between two paired or unpaired groups, respectively. Comparisons of differences between groups in the number of challenges before viral acquisition were assessed using the log-rank (Mantel-Cox) test of the discrete-time proportional hazards model. The average per-risk challenge of viral acquisition was estimated as the total number of observed infections divided by the number of administered challenges. Correlation analyses were performed using the non-parametric Spearman rank correlation method with exact permutation and approximate two-tailed P values calculated for the number of pairs≤17 and >17, respectively.

### Example 1

### Mucosal Treatment with SAMT-247 Protects 80% of Vaccinated Female Macaques from SIV_{mac251} Mucosal Acquisition

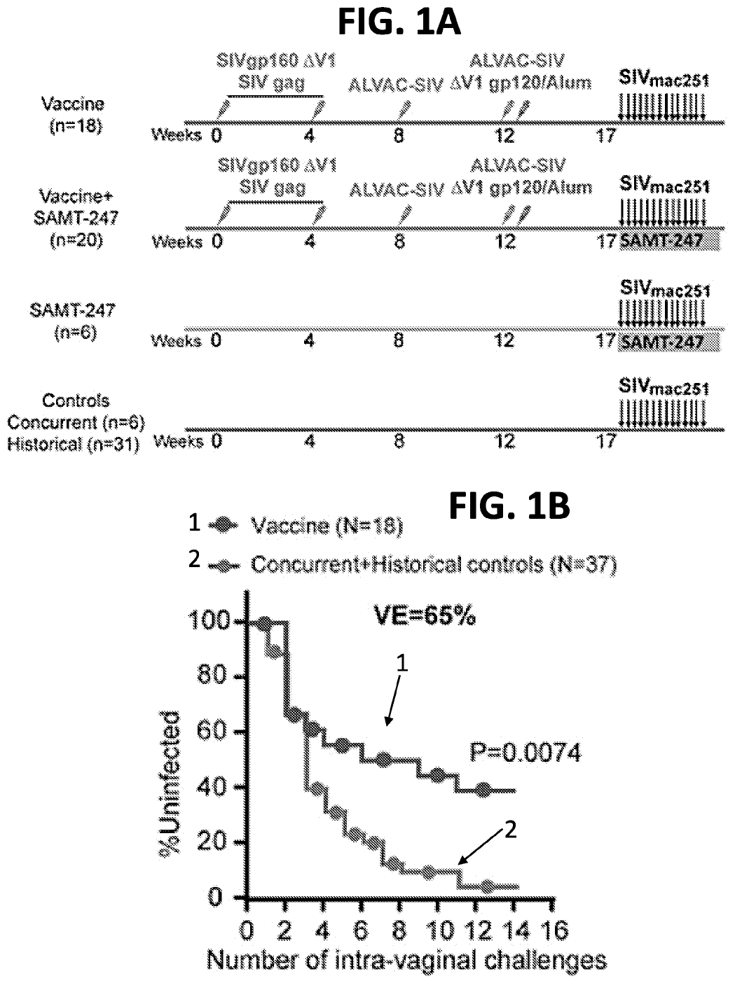

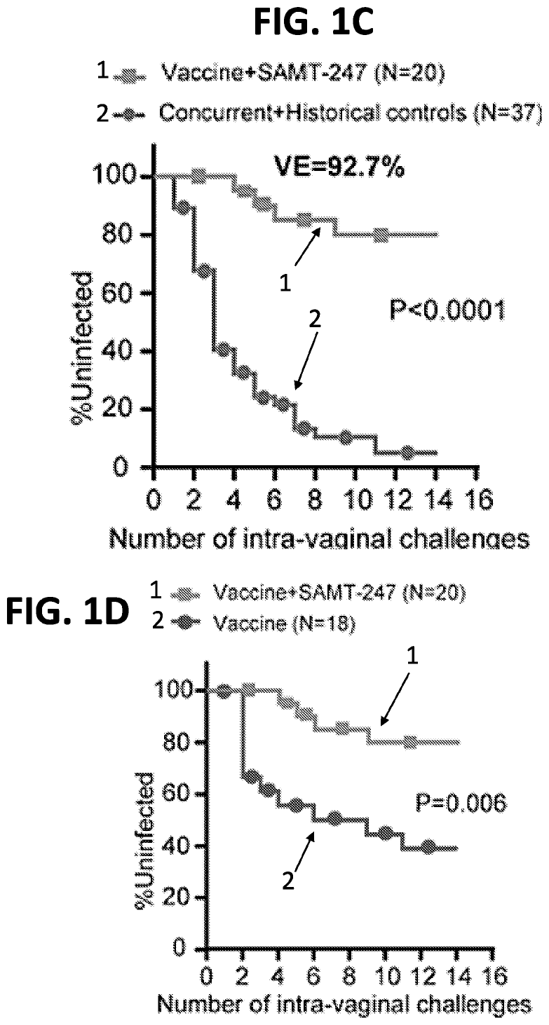

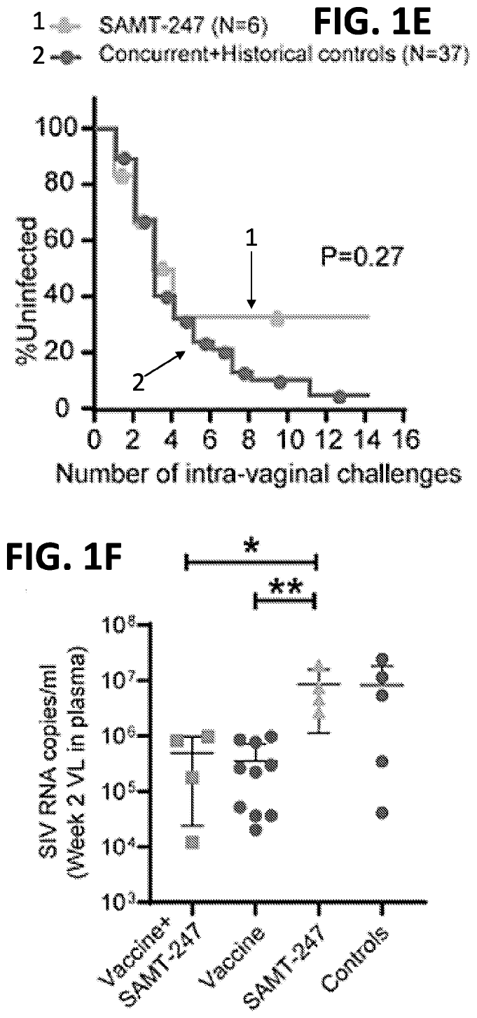

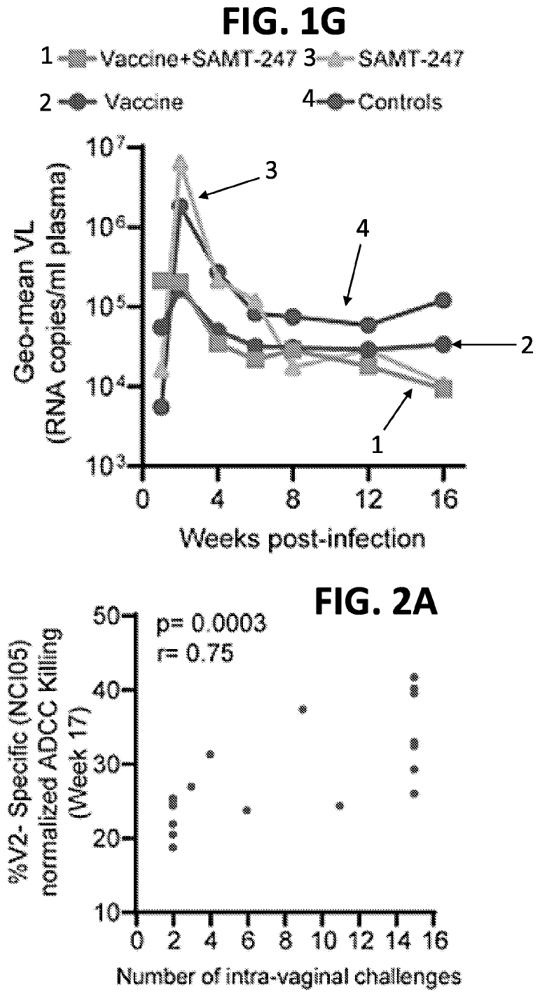

A study in macaques was designed that was powered to dissect the differences between vaccination alone and vaccination plus SAMT-247 treatment based on the premise that ΔV1 DNA/ALVAC-SIV /ΔV1gp120/alum vaccination reproducibly decreases the risk of SIV_{mac251} acquisition by 60-70% (Silva de Castro, I., et al., iScience 24, 102047 (2021). The vaccine regimen was administered to thirty-eight female macaques; the study population included the animal disclosed in U.S. Provisional Application No. 63/228,707, filed Aug. 3, 2021, which is incorporated herein by reference. Five weeks after the last immunization (week 17), all animals were exposed weekly to intravaginal SIV_{mac251} challenges for up to 14 consecutive weeks. Four hours prior to each challenge exposure, 20 vaccinated animals were treated with 0.8% SAMT-247 in HEC gel, and the remaining 18 animals with HEC gel only. Two additional groups of non-immunized animals (6 each) were treated either with SAMT-247/HEC gel or HEC gel at 4 hours prior to viral exposure as controls. All animals were challenged until infection was confirmed by nanodroplet PCR (FIG. 1A). The study was designed to include SIV acquisition data from 31 historical controls (see Methods) challenged with the same stock of virus in the same animal facility. As expected, no difference in virus acquisition was observed between concurrent and historical controls. Vaccine alone decreased the risk of virus acquisition by 65% (p=0.0074; FIG. 1B), consistent with prior data (Bissa, M.e.a. HIV vaccine candidate efficacy mediated by cyclic AMP-dependent efferocytosis and V2-specific ADCC. Submitted (2022)). Strikingly, however, the vaccine+SAMT-247 combination afforded a 92.7% reduction in the risk of virus acquisition when compared to all controls (p=0.0001; FIG. 1C), as well as concurrent and historical controls separately (p=0.0002 and p=0.0001 respectively) and differed significantly from the vaccinated only group (p=0.006; FIG. 1C). The vaccine+SAMT-247 combination protected 16 of 20 animals (80%) from infection. In contrast, treatment with SAMT-247 alone did not significantly decrease the risk of virus acquisition when compared to controls separately or combined (FIG. 1E). The vaccinated animals that became infected with or without SAMT-247 treatment had lower peak virus levels than SAMT-247 treated controls, whereas no difference in peak virus levels was observed between controls and SAMT-247 only groups (FIG. 1F). Since only four animals in the vaccine+SAMT-247 treated group became infected, the statistical power for viral load comparison over time was low. Overall, the control group maintained a comparatively higher geometric mean virus level compared to the other macaque groups, though not significantly (FIG. 1G).

### Example 2

### SAMT-247 Augments V2-Specific ADCC and Cytotoxic NKG2A⁺ Cells

Reproducible immune correlates of reduced risk elicited by the ΔVIDNA/ALVAC/ΔV1gp120/alum vaccine platform include systemic V2-specific ADCC, mucosal envelope specific NKp44⁺ cells producing IL-17, CD14⁺ monocytes mediating efferocytosis, and Th1/Th2 cells expressing no or low levels of CCR5 (Silva de Castro, I., et al., iScience 24, 102047 (2021); Vaccari, M., et al., Nat Med 22, 762-770 (2016); Vaccari, M., et al. Nat Med 24, 847-856 (2018); Bissa et al, supra, (2022)). Even though all animals were vaccinated with the identical regimen, immune correlates of risk were tested separately in the two vaccinated groups to validate equivalent vaccine immunogenicity.

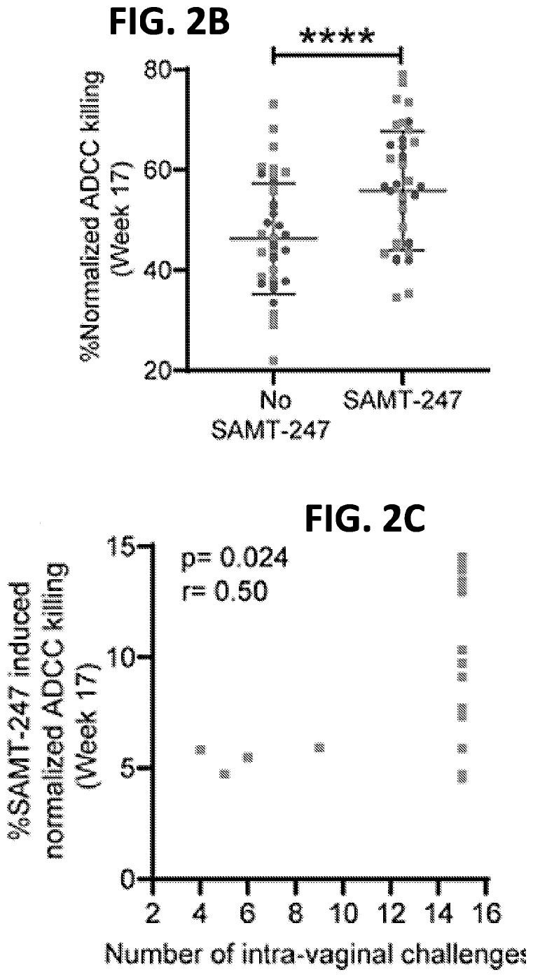

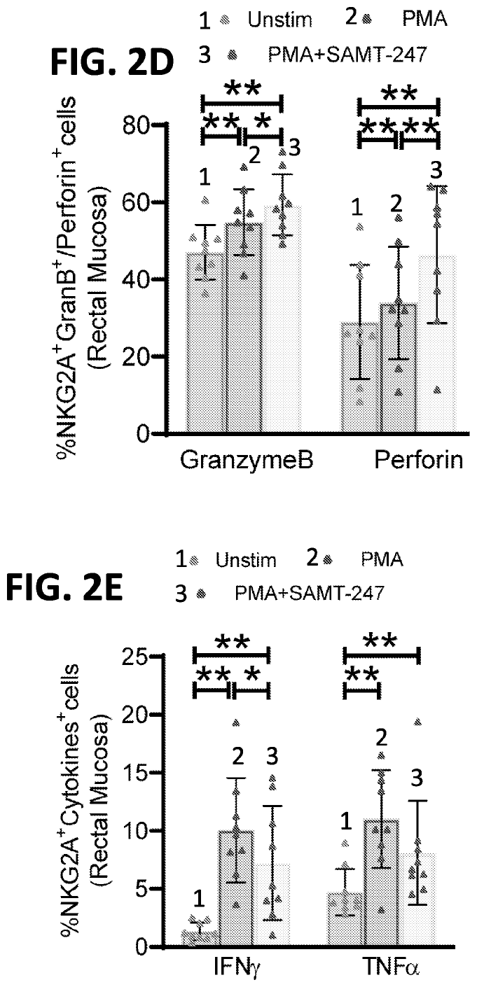

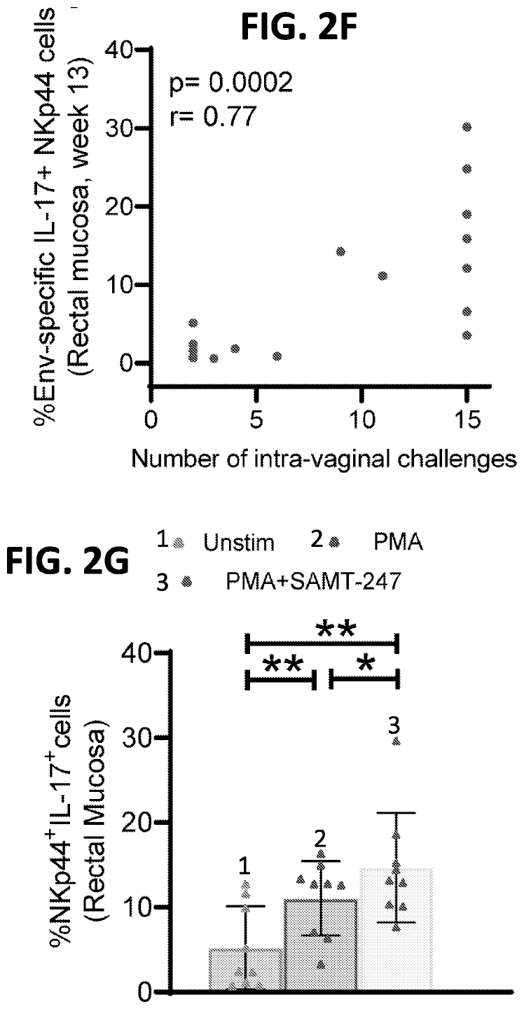

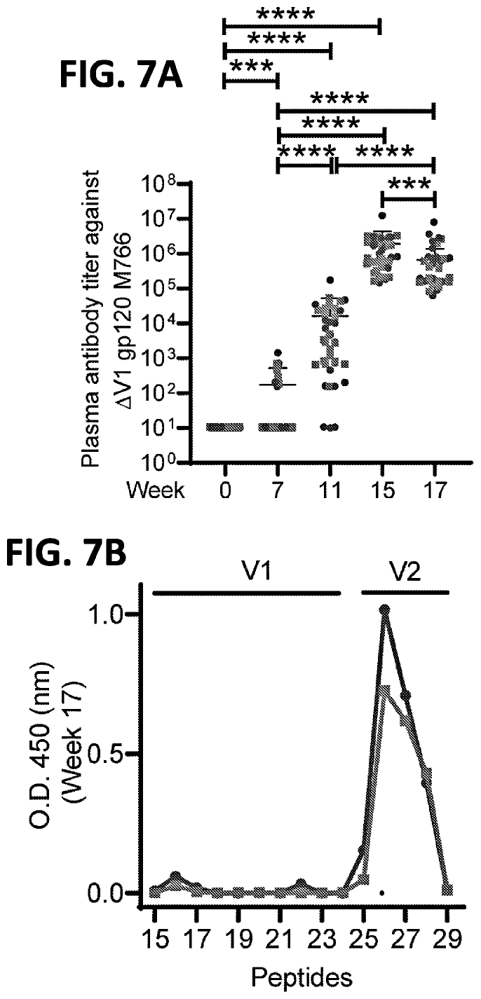

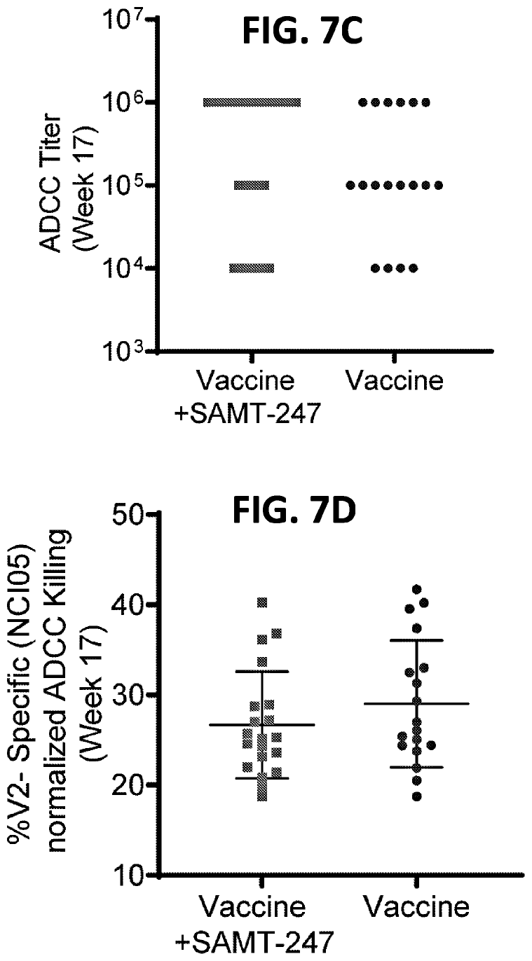

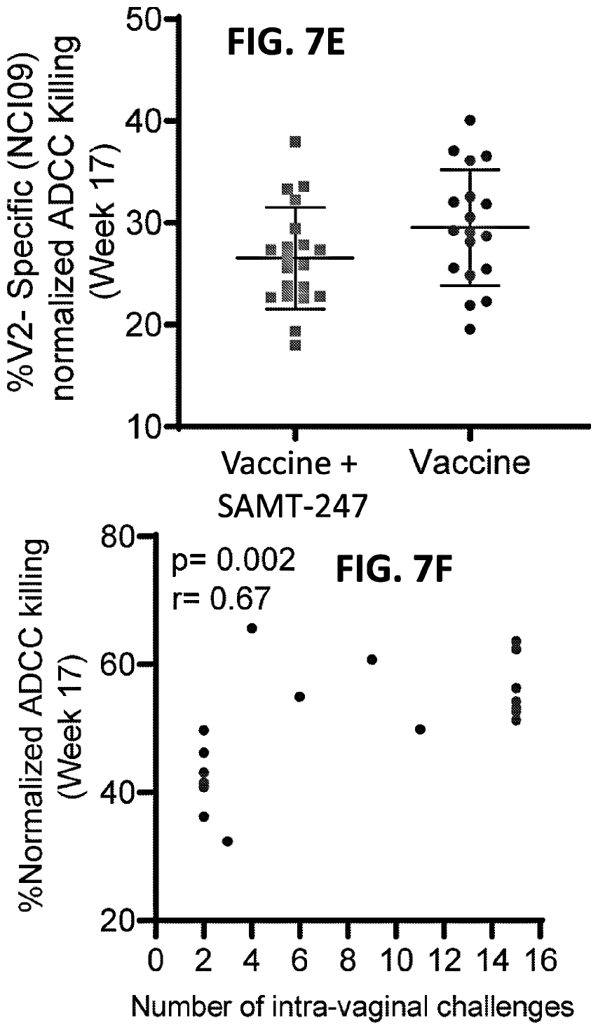

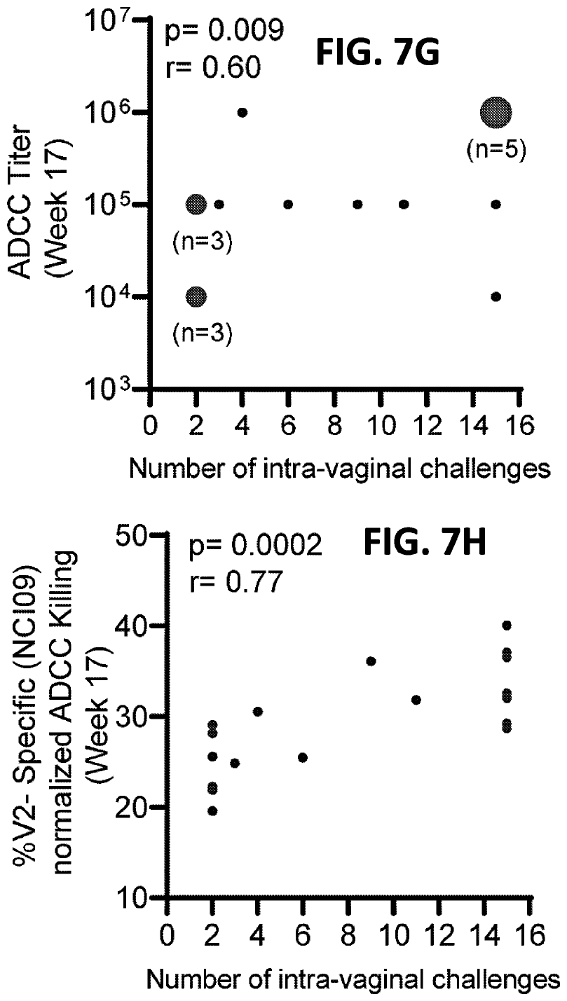

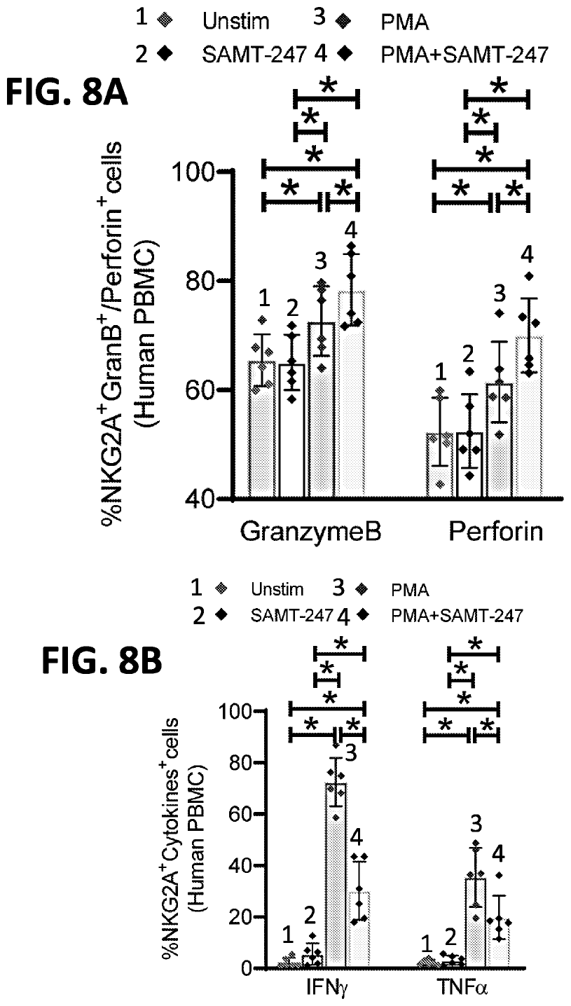

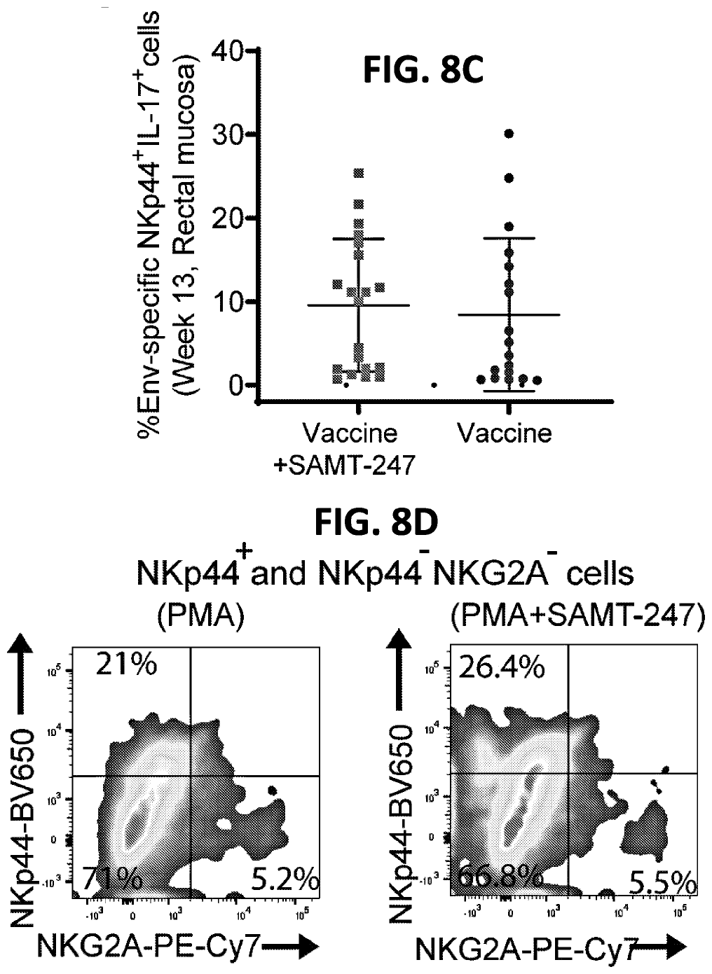

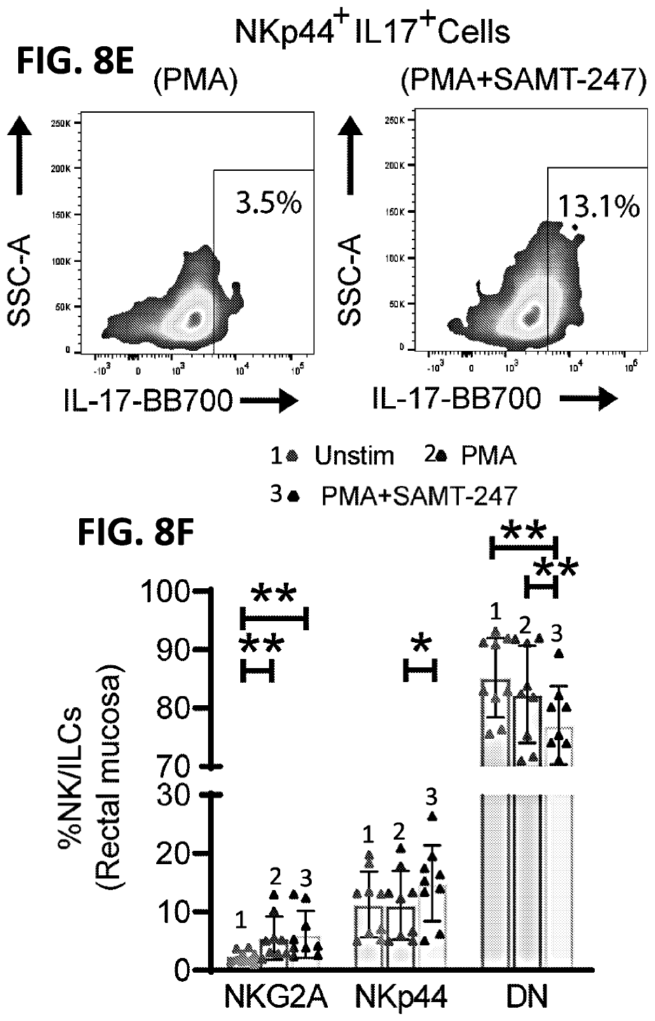

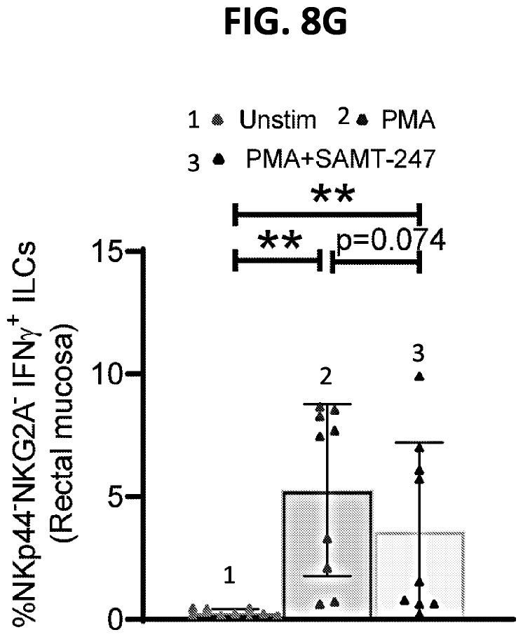

The levels of systemic antibodies to ΔV1gp120 and V2 peptides (FIG. 7A, 7B) and ADCC titer and V2-specific ADCC did not differ in the vaccinated treated or untreated groups, as expected (FIGS. 7C-7E). Surprisingly, however, ADCC activity and titers (FIG. 7F, 7G) as well as V2-specific ADCC correlated with decreased risk of SIV_{mac251} acquisition in the vaccinated only group, and not in the vaccine+SAMT-247 animals (FIG. 2A and FIG. 7H), raising the hypothesis that SAMT-247 treatment may have affected mucosal ADCC in vivo at the virus exposure mucosal site. Pretreatment of effector human PBMCs with SAMT-247 prior to the ADCC assay demonstrated that SAMT-247 significantly augments ADCC activity mediated by the plasma of all vaccinated animals (FIG. 2B). Strikingly, in the vaccinated+SAMT-247 treated animals, the difference in ADCC activity measured in the absence or presence of SAMT-247 in vitro restored the correlation of ADCC with a decreased risk of virus acquisition (FIG. 2C), suggesting that topical administration of SAMT-247 may have augmented mucosal NK effector function. The ADCC assay used in these studies was based on the effector activity of human PBMCs in the presence of antibodies from the immunized animals recognizing target cells coated with SIV ΔV1 gp120 protein. It was confirmed that in vitro treatment of human PBMCs with SAMT-247 did not affect the expression of granzyme, perforin, or cytokines in the absence of PMA stimulation; in contrast, in the presence of PMA, SAMT-247 increased the percentage of NKG2A⁺ NK cells expressing granzyme B and perforin (FIG. 8A) and interestingly decreased the percentage of IFN-γ and TNF-α producing NKG2A⁺ cells (FIG. 8B). Next, it was assessed whether SAMT-exerted the same effect on mucosal NK cells following isolation of macaque mononuclear cells from rectal mucosa. It was found that as in the case of blood NK cells in humans, SAMT-247 increased NKG2A⁺ cells expressing granzyme B and perforin and decreased both IFN-γ and TNF-α production also in macaque mucosal NK cells (FIGS. 2D, 2E).

### Example 3

### SAMT-247 Increases Mucosal NKp44⁺ IL-17⁺ Cell Frequency and Decreases NKG2A⁻ NKp44⁻ Producing IFN-γ

Mucosal NKp44⁺cells produce IL-17 and IL-22 to maintain the integrity of mucosal epithelia. In prior studies, SIV envelope specific NKp44⁺ IL-17+ cells correlated with decreased risk of virus acquisition (Vaccari, M., et al., Nat Med 22, 762-770 (2016); Rahman, M. A., et al, J Immunol 203, 2459-2471(2019)). The frequency of protective envelope-specific mucosal NKp44⁺ cells producing IL-17¹² did not differ in the vaccinated/SAMT-247 treated or untreated groups, as expected, since at this time point no SAMT-247 was administered (FIG. 8C). However, their frequency correlated significantly with a decreased risk of virus acquisition in the vaccinated only group (FIG. 2F), suggesting that SAMT-247 treatment may have modulated this response. To test this hypothesis mucosal cells isolated from 9 macaques vaccinated with the DNA/ALVAC/gp120/alum vaccine were exposed in vitro to SAMT-247. It was found that SAMT-247 treatment augmented the percentage of NKp44⁺ cells producing IL-17 (FIG. 2G, FIGS. 8D-8E), supporting the hypothesis that in vivo mucosal treatment with SAMT-247 may have locally augmented the function of NKp44⁺ cells, thereby augmenting vaccine efficacy.

In prior studies it was found that the NKG2A⁻NKp44⁻ population of mucosal cells producing IFN-γ was associated with an increased risk of virus acquisition (Vaccari, M., et al., Nat Med 22, 762-770 (2016); Rahman, M. A., et al, J Immunol 203, 2459-2471(2019)). In the present studies, it was determined that in vitro SAMT-247 treatment of mucosal cells decreased the frequency of the NKG2A⁻NKp44⁻ population and tended to decrease their ability to produce IFN-γ (FIGS. 8F, 8G).

Collectively, these data demonstrate that SAMT-247 synergizes with vaccination by increasing protective NKp44⁺/IL-17⁺ cell responses and by decreasing the frequency of NKG2A⁻ NKp44⁻ producing IFN-γ (Table 1).

TABLE 1
Effect of SAMT-247 alone on NK cell and monocyte function.

| | Cytokine | Function |
|---|---|---|
| NKG2A⁺ | >Granzyme/Perforin | >ADCC |
| Blood and | <IFN-γ * | |
| mucosa PMA | <TNF-α * | |
| NKp44⁺ | >IL-17 | >Epithelium |
| Mucosa | | homeostasis |
| PMA | | |
| Monocytes | >IL-10 * | >Efferocytosis |
| unstimulated | | |
| or +gp120 peptides | | |

* Zinc dependence

### Example 4

### SAMT-247 increases CD14⁺ Efferocytosis

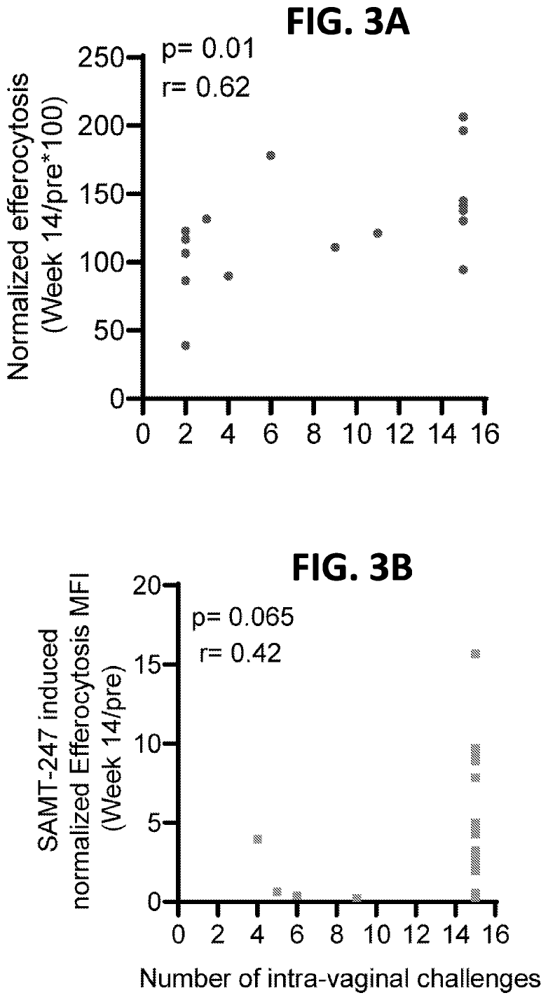

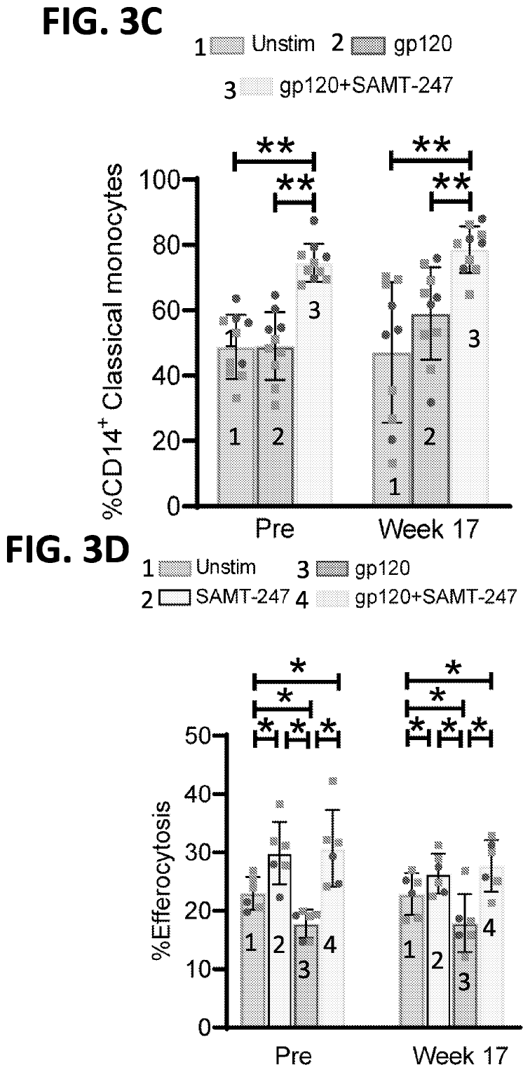

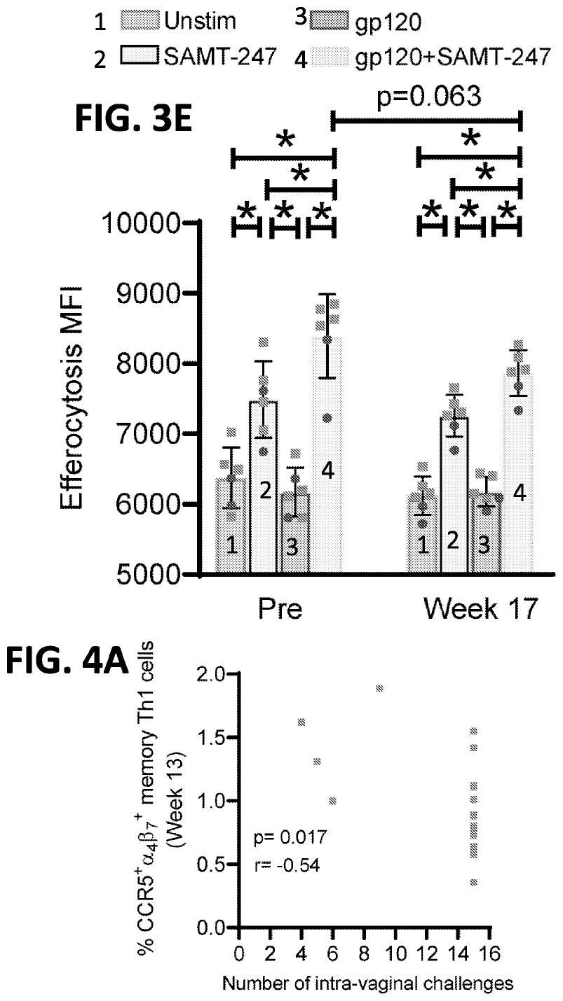

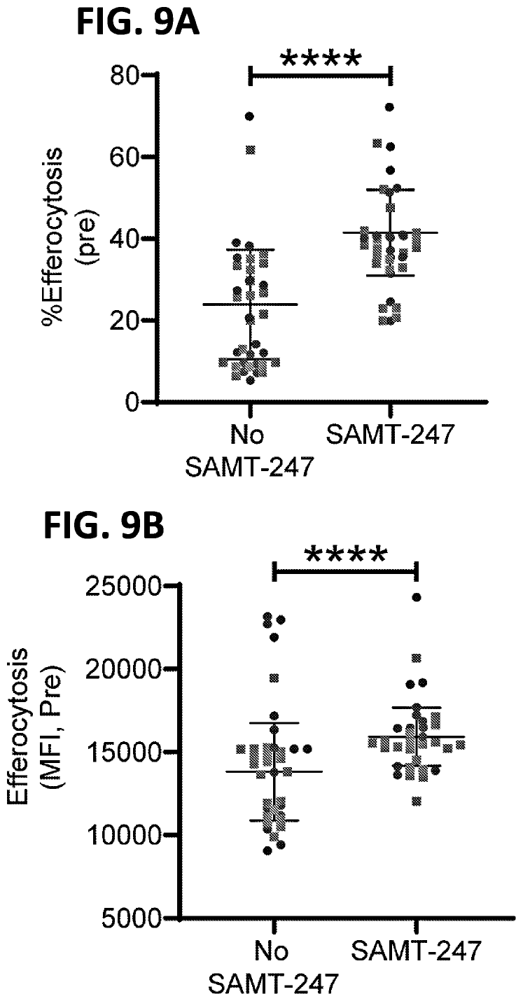

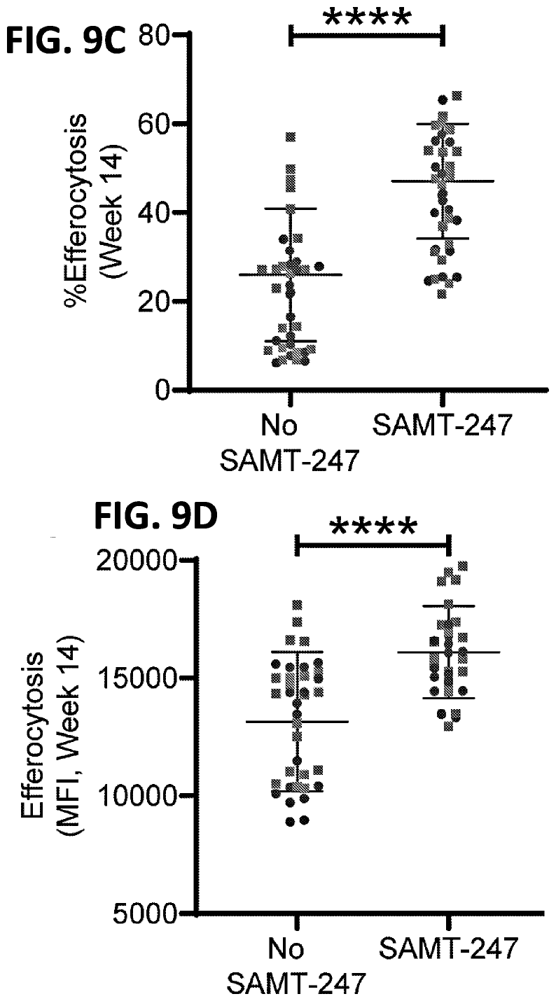

Efferocytosis is an innate CD14⁺ monocyte response essential for the clearance of apoptotic cells, maintenance of tissue homeostasis and eradication of pathogens (Henson, P. M., Annu Rev Cell Dev Biol 33, 127-144 (2017)). CD14⁺ cell-associated efferocytosis, like ADCC, is a reproducible immune correlate of reduced risk of SIV_{mac251} acquisition following vaccination with the DNA/ALVAC/gp120/alum regimen. Vaccine induced CD14⁺ cell-mediated efferocytosis correlated with a reduced risk of virus acquisition (R=0.62; p=0.01) in the vaccinated group as expected (FIG. 3A), but not in the vaccinated/SAMT-247 treated group (data not shown). This finding raised the hypothesis that SAMT-247 may have affected the function of macrophages at mucosal site. To investigate indirectly this hypothesis, an efferocytosis assay was performed with CD14+cells from blood of vaccinated animals in the presence and absence of SAMT-247. It was found that SAMT-247 augmented both the percentage of CD14⁺ cells engulfing apoptotic cells, as well as the per-cell amount of engulfed apoptotic cells (measured as Mean Fluorescent Intensity, MFI) within CD14⁺ cells collected before (pre) and at two weeks following the last immunization (FIGS. 9A-9D). Differences in the ratio of the ability of CD14⁺ cells to engulf apoptotic cells before and following vaccination revealed by SAMT-247 treatment in vitro trended with a reduced risk of viral infection in the animals treated in vivo with SAMT-247 (R=0.42; p=0.065; FIG. 3b). To further investigate the basis of SAMT-247's effect on CD14⁺ cell function and its relation to the protection from infection in the macaque model, PBMCs from animals prior to and following vaccination (week 17) were stimulated with overlapping peptides encompassing the entire gp120 envelope protein to mimic an in vitro host response at the time of virus encounter. Stimulation of PBMCs with gp120 peptides+SAMT-247 for 12 hours resulted in an increased percentage of CD14⁺ cells at both time points, whereas gp120 peptides alone had no significant effect (FIG. 3C). Sorted monocytes from PBMCs cultured in the different conditions were next tested in efferocytosis assays. Strikingly, while stimulation with gp120 peptides significantly decreased the percentage of CD14⁺ cells engulfing apoptotic neutrophils, SAMT-247 treatment reconstituted the percentage of efferocytotic cells and the per-cell amount of cells engulfed (MFI) (FIGS. 3D, 3E).

The data demonstrate that SAMT-247 synergizes with vaccine induced responses at multiple levels: by increasing NKG2A⁺ cytotoxic function and V2 specific protective ADCC; by increasing the functionality of protective NKp44⁺ cells and CD14⁺ cells, both responses essential to maintain tissue homeostasis and curb inflammation; and by decreasing the frequency of mucosal inflammatory NKG2A⁻NKp44⁻ cells producing IFN-γ, associated with increased risk of virus acquisition (Table 1).

### Example 5

### SAMT-247 Modulates CCR5 and TNF-α Expression in CD4⁺ Cells

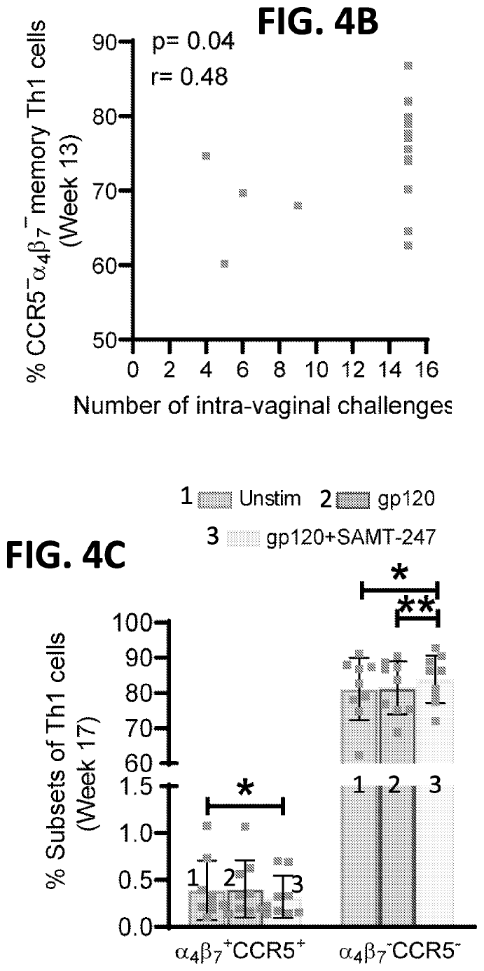

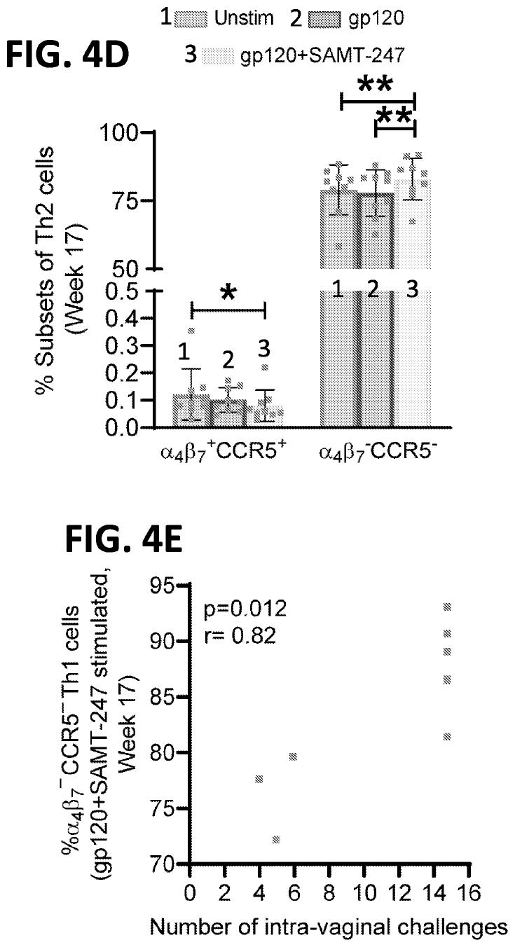

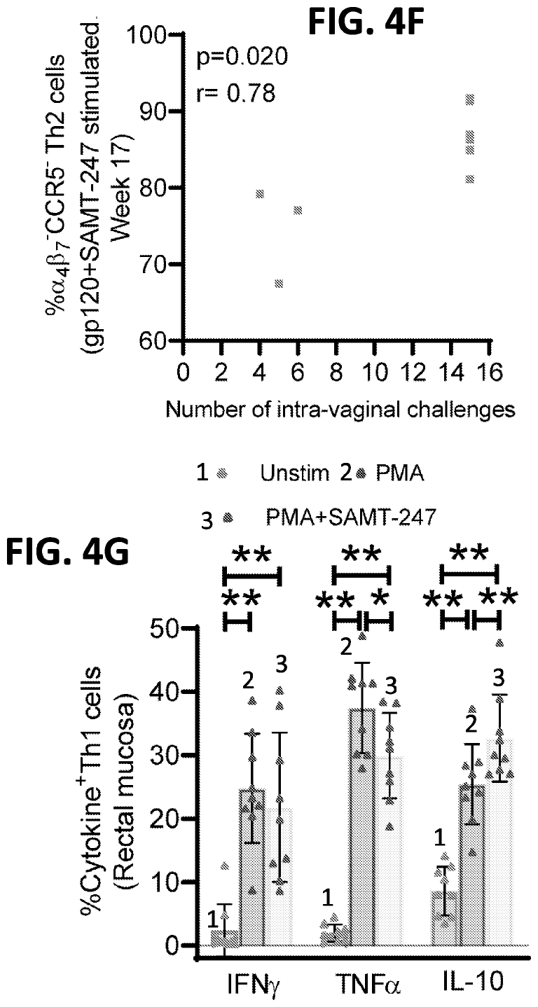

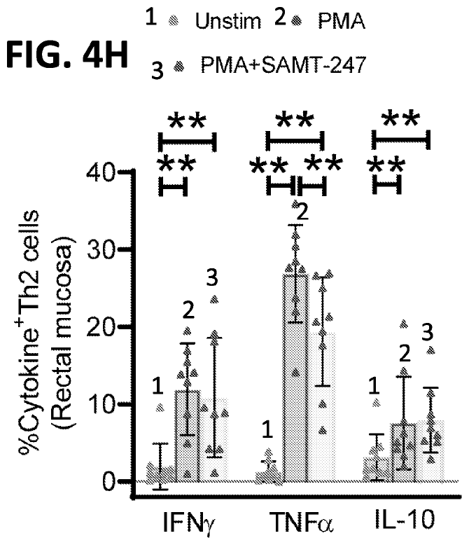

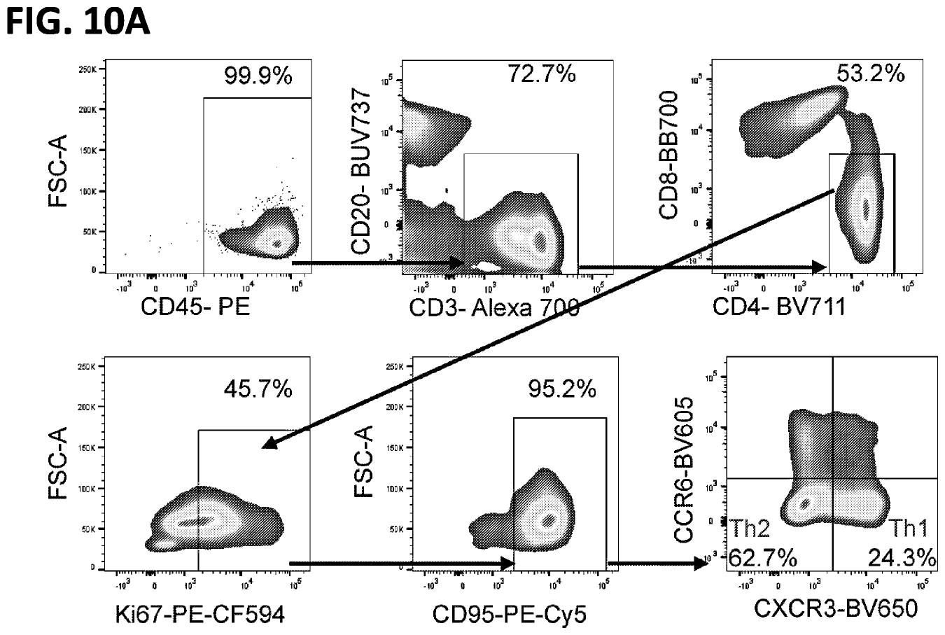

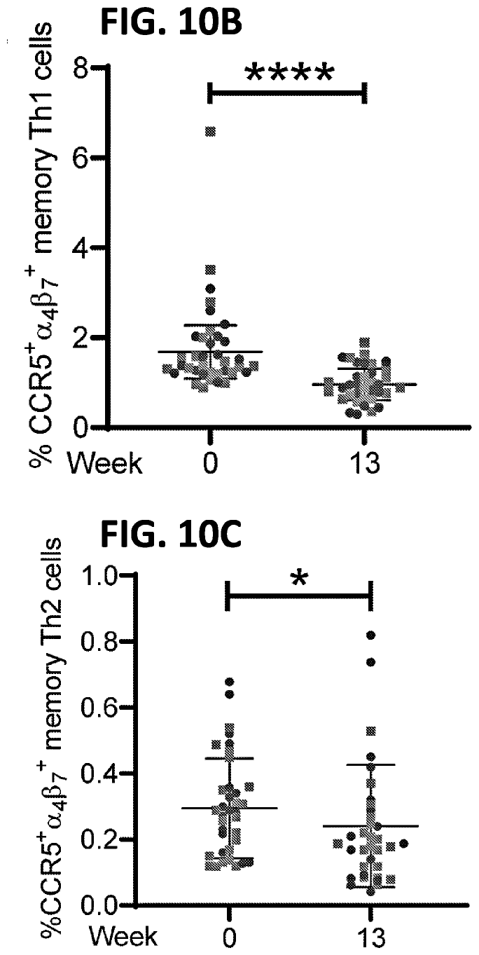

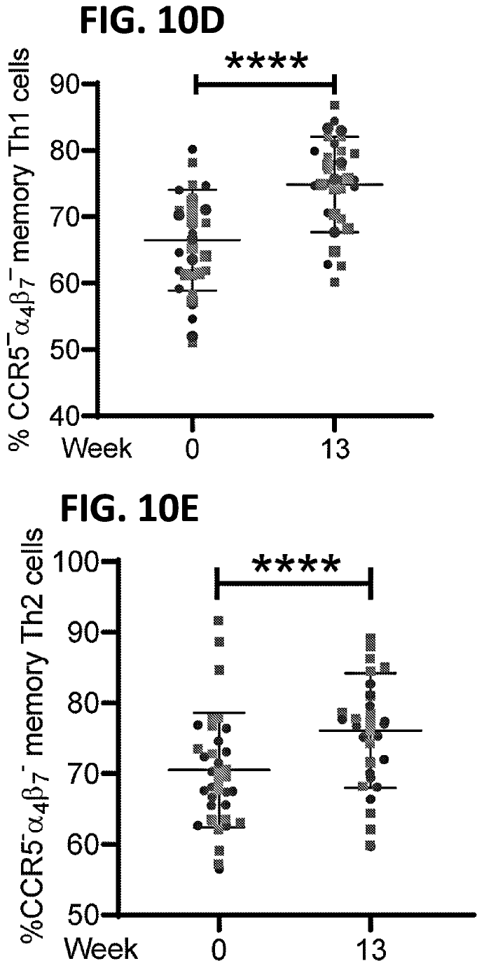

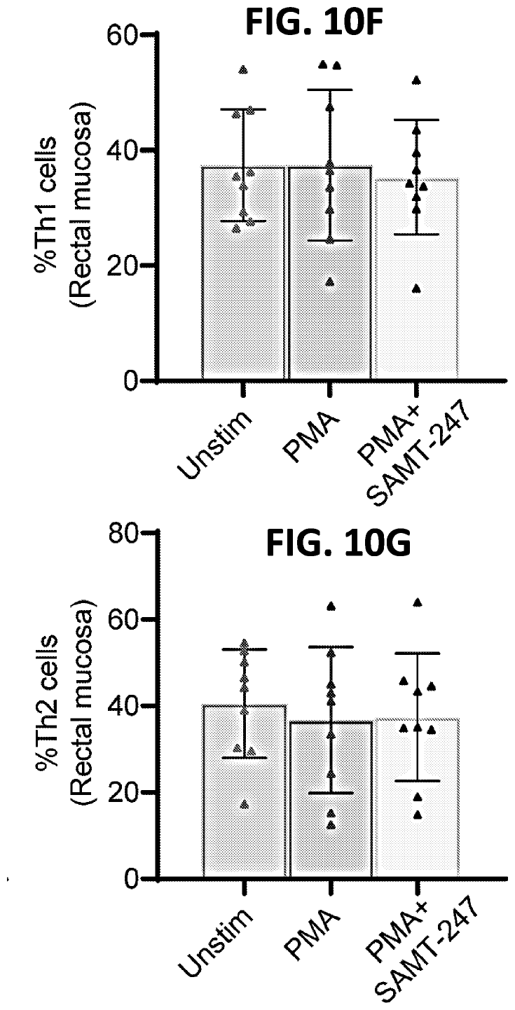

Vaccine-induced gut homing activated α₄β₇⁺ CD4 cells expressing the SIV/HIV co-receptor CCR5 (α₄β₇⁺CCR5⁺ CD4⁺ cells) have been associated with an increased risk of virus acquisition (Cicala, C., et al., Proc Natl Acad Sci U S A 106, 20877-20882 (2009); Kader, M. et al., J Med Primatol 38 Suppl 1, 24-31 (2009)). Vaccination with the ΔVIDNA/ALVAC/ΔV1gp120/alum regimen significantly decreased the frequency of vaccine-induced (Ki67⁺) α₄β₇⁺CCR5+ memory Th1 (CD4⁺α₄β₇⁺CCR5⁺CCR6⁻CXCR3⁺Ki67⁺CD95⁺) and Th2 (CD4⁺α₄β₇⁺CCR5⁺CCR6⁻CXCR3⁻Ki67⁺CD95⁺) cell phenotypes (p<0.0001 and p=0.014, respectively) and increased significantly the frequency of α₄β₇ ⁻CCR5⁻CD4⁺memory Th1 and Th2 cells (p<0.0001 and p<0.0001, respectively; FIGS. 10A-10E). In the vaccinated+SAMT-247 treated group (but not in the vaccine only group), the frequency of α₄β₇⁺CCR5⁺CD4⁺ Th1 memory cells one week after the last immunization (week 13) correlated with increased risk of acquisition (r=−0.54; p=0.017), whereas Ki67⁺CD4⁺α₄β₇ CCR5⁻ at the same timepoint correlated with decreased risk (R=0.48; p=0.04; FIGS. 4A, 4B). Interestingly, the correlation with α₄β₇⁻CCR5⁻CD4+ memory Th1 cells found at week 13 was lost at week 17, the time of challenge exposure. It was hypothesized that mucosal treatment with SAMT-247 may have decreased local mucosal T-cell activation. To investigate this, PBMCs from vaccinated animals (week 17) were stimulated with overlapping peptides encompassing the entire gp120 envelope protein to mimic host response at the time of virus encounter. SAMT-247+gp120 peptides decreased the frequency of both vaccine-induced (Ki67⁺) Th1 and Th2 α₄β₇⁺CCR5⁺ cells and increased Th1 and Th2 α₄β₇⁻CCR5⁻ cells (FIGS. 4C, 4D). Strikingly, the percentage of change of α₄β₇⁻CCR5⁻ subsets within Th1 and Th2 cells correlated with delayed virus acquisition in vivo (FIGS. 4E, 4F). To relate this finding of CD4⁺ cells in the blood of the vaccinated animals to mucosal compartments, the effect of SAMT-247 treatment on mucosal Th1 and Th2 cells following PMA stimulation was investigated. The overall mucosal Th1 and Th2 cell frequencies did not change upon PMA stimulation (FIGS. 10F, 10G). However, SAMT-247 treatment was associated with a significant decrease in TNF-α production in both Th1 and Th2 cells and with an increase in IL-10 production in Th1 cells (FIGS. 4G, 4H and Table 2).

TABLE 2
Effect of PMA or PMA + SAMT-
247 on mucosal Th1 or Th2 cytokines.

| | | Unstim vs PMA + | PMA vs PMA + |
|---|---|---|---|
| | Unstim vs PMA | SAMT-247 | SAMT-247 |
| | Stimulation | Stimulation | Stimulation |
| | Cytokines | Cytokines | Cytokines |
| Th1 | >IFN-γ | >IFN-γ | |
| | >TNF-α | >TNF-α | <TNF-α |
| | >IL-10 | >IL-10 | >IL-10 |
| Th2 | >IFN-γ | >IFN-γ | |
| | >TNF-α | >TNF-α | <TNF-α |
| | >IL-10 | >IL-10 | |

### Example 6

### Effects of Chelation of Free Divalent Zinc on NK, Monocytes, and T-Cells

As administered in the study, SAMT-247 alone did not significantly decrease the risk of virus acquisition, suggesting additional drug activity since its combination with vaccination protected nearly all animals from infection. It was hypothesized that SAMT-247 may have augmented vaccine-induced immunity by affecting zinc distribution in immune cells, since zinc is a master regulator of immunity (Read, S. A., et al., Adv Nutr 10, 696-710 (2019)).

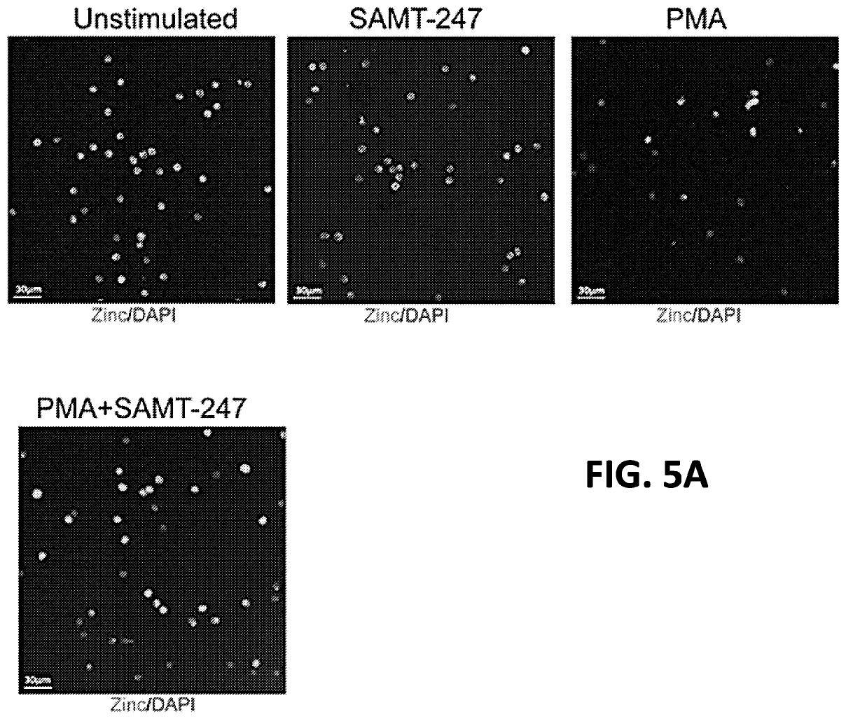

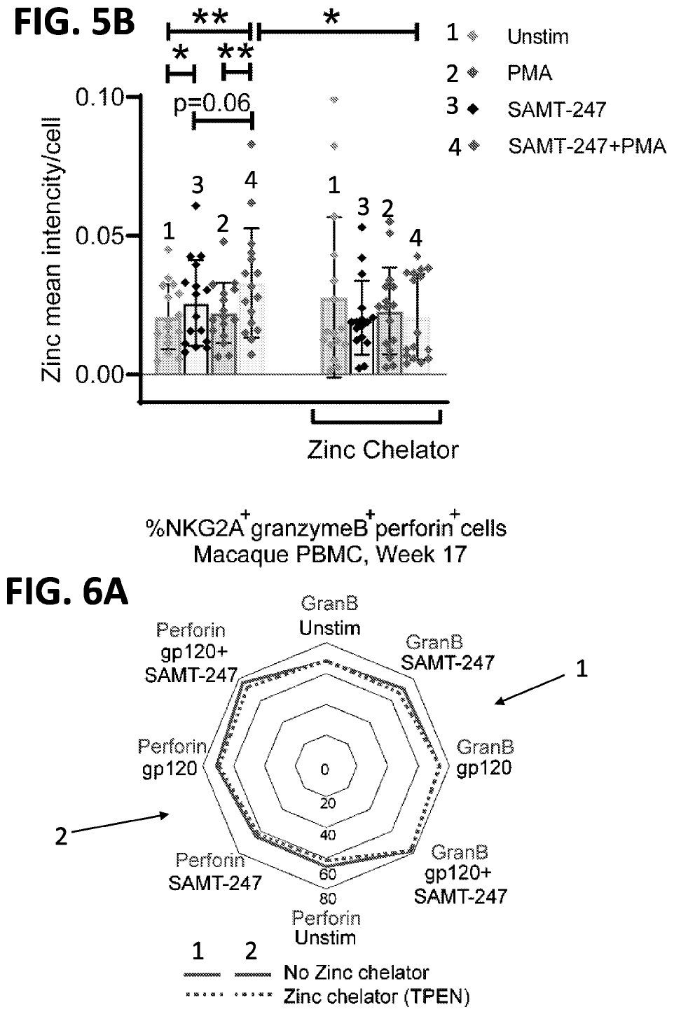

The spectrum of SAMT-247 effects on NK, monocytes, and T-cell functions observed above suggested that SAMT-247 may affect these immune responses by ejection of zinc (Zn), a master regulator of immunity. Confocal microscopy was used to measure the effect of SAMT-247 on cellular zinc using human NK cells stimulated with PMA in the presence or absence of SAMT-247, and with or without a zinc chelator. PMA+SAMT-247 treated cells had significantly brighter zinc staining compared to unstimulated and PMA stimulated cells, and also showed a trend of higher intensity compared to SAMT-247 stimulated cells (FIGS. 5A, 5B). In the presence of zinc chelator, PMA+SAMT-247 stimulated cells showed significantly lower zinc intensity compared to those not treated with chelator (FIG. 5B), demonstrating that SAMT-247 co-stimulatory activity is dependent on the availability of divalent free zinc following PMA stimulation.

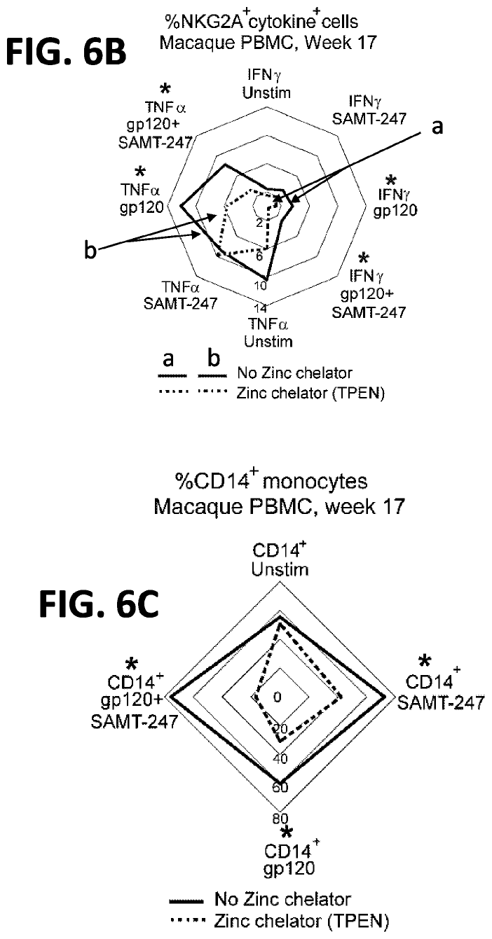

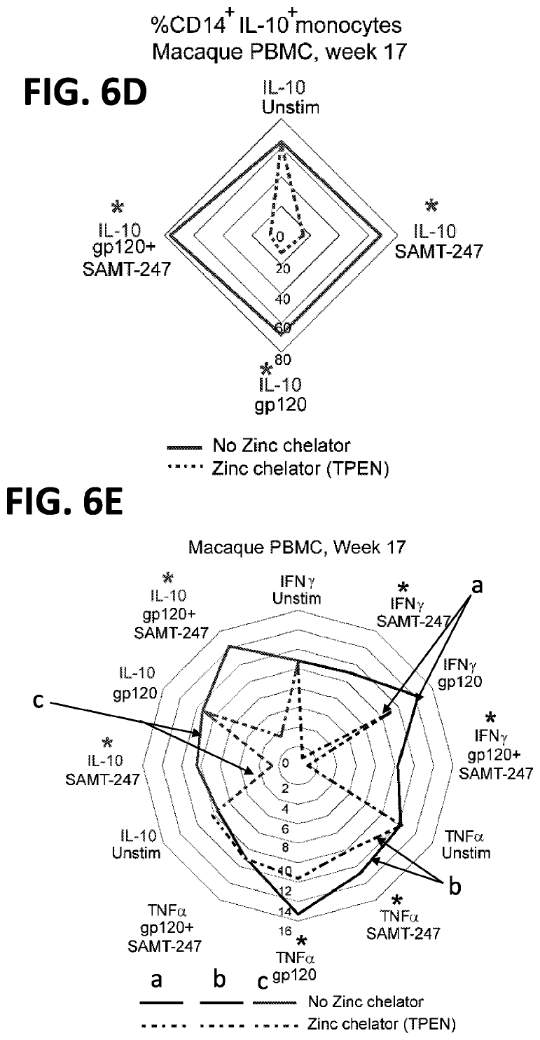

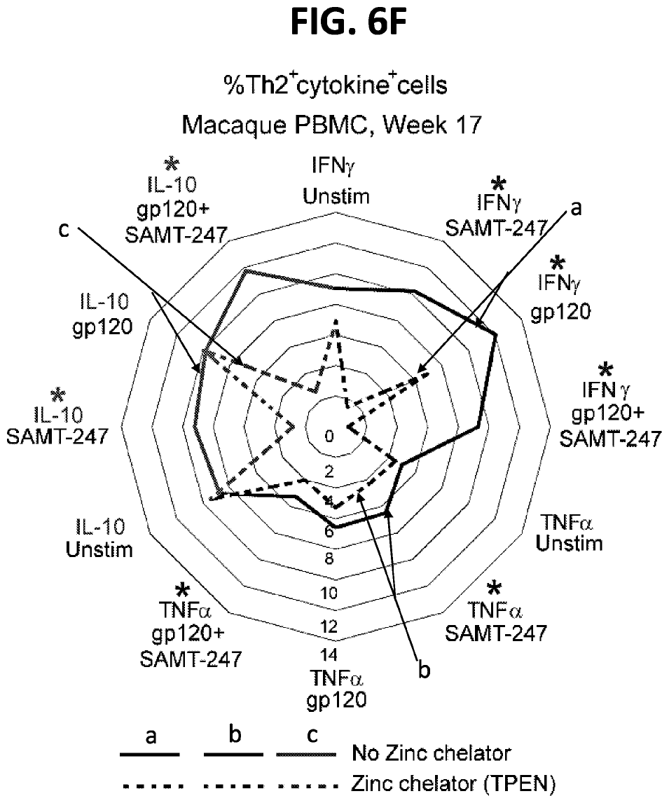

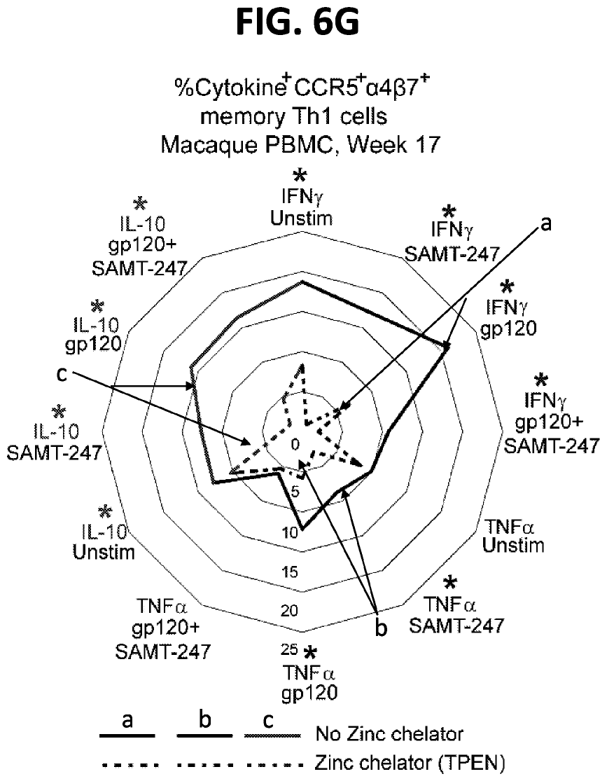

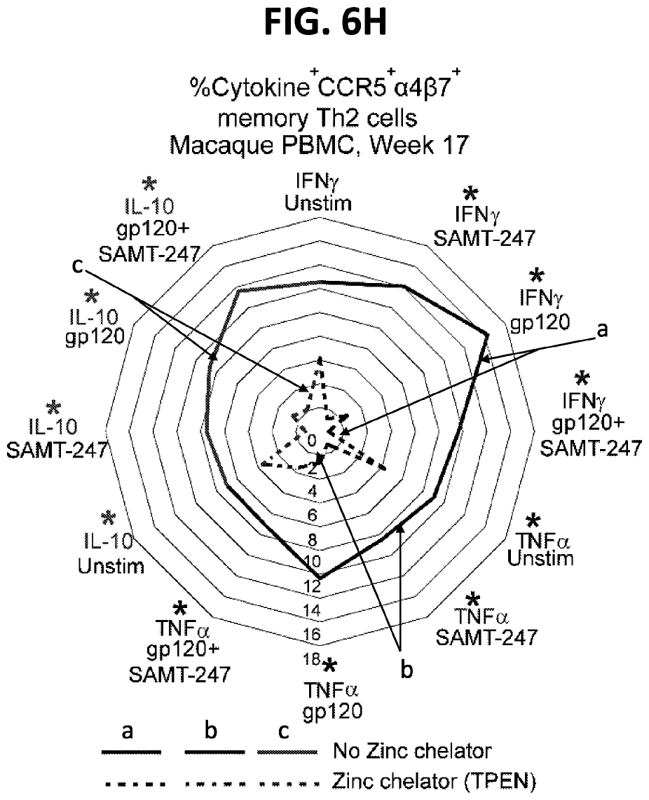

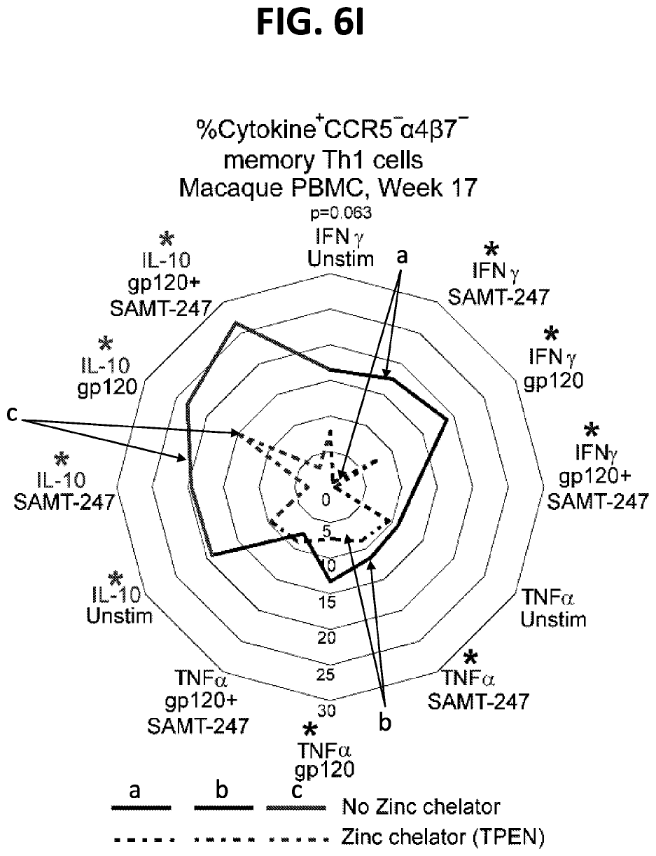

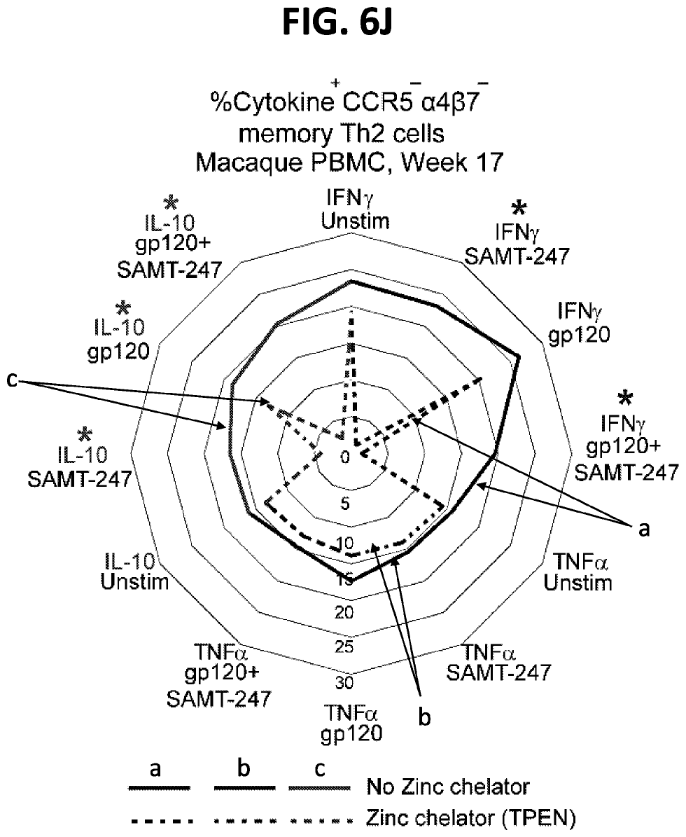

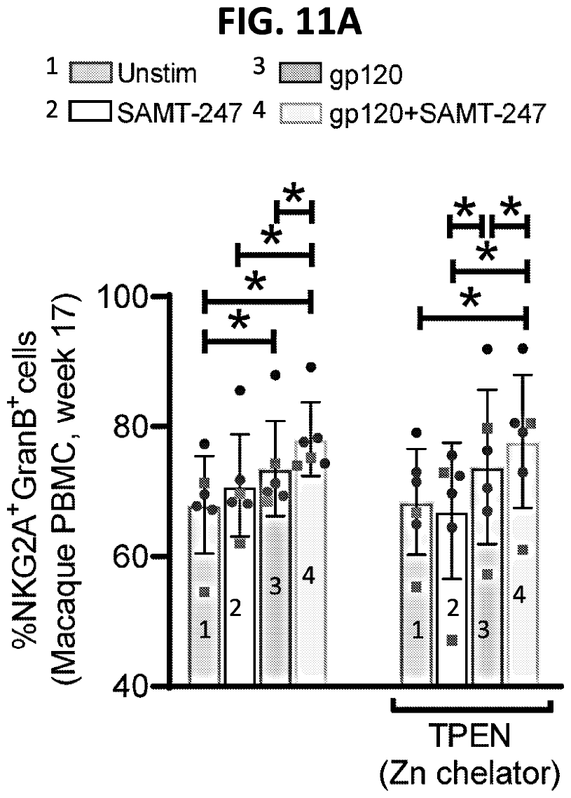

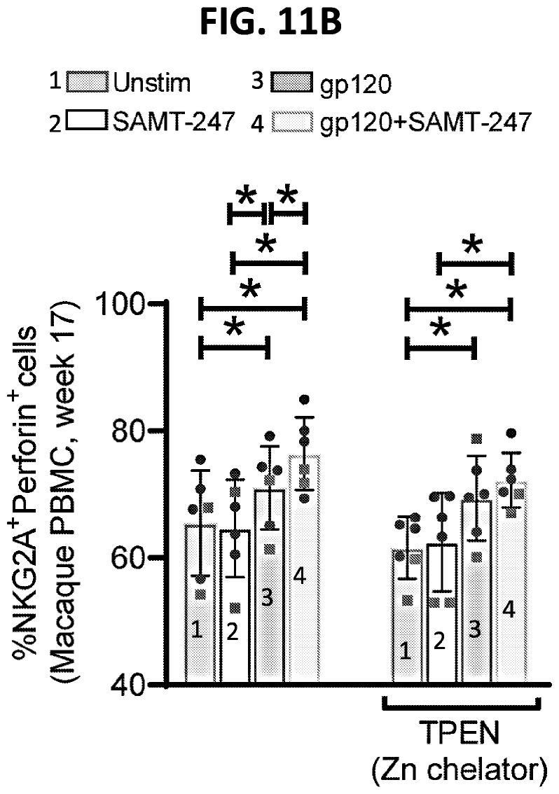

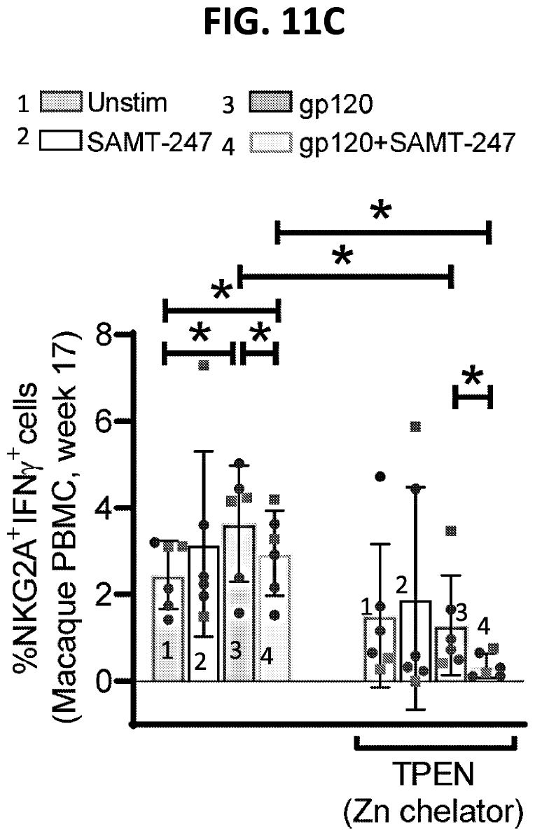

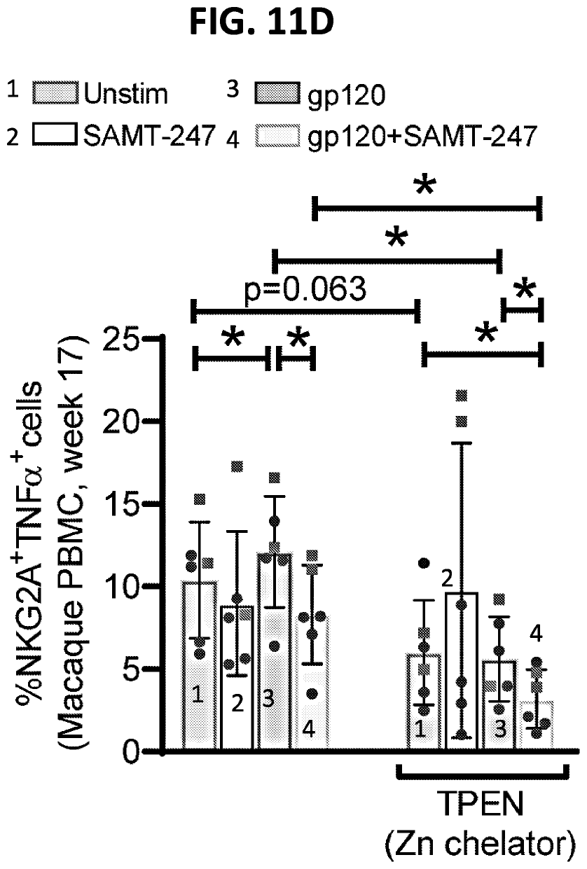

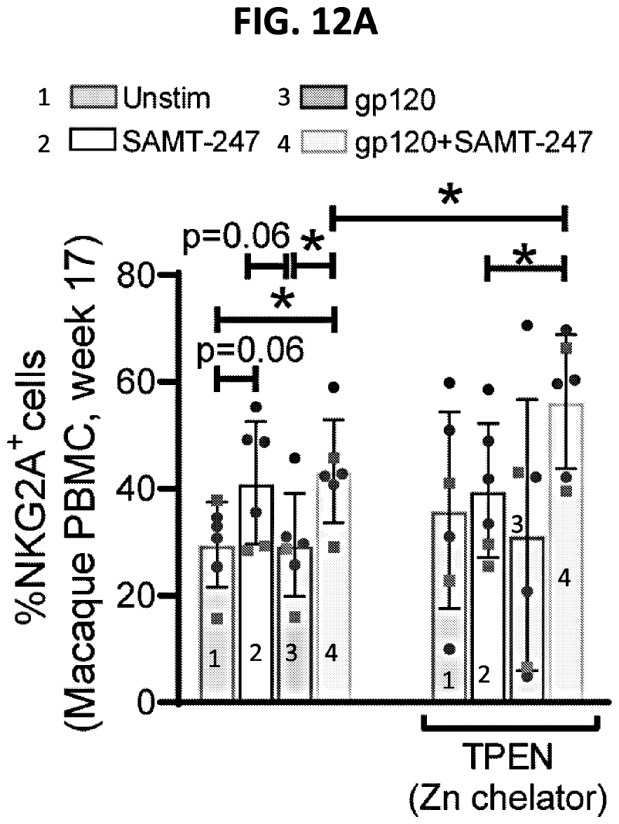

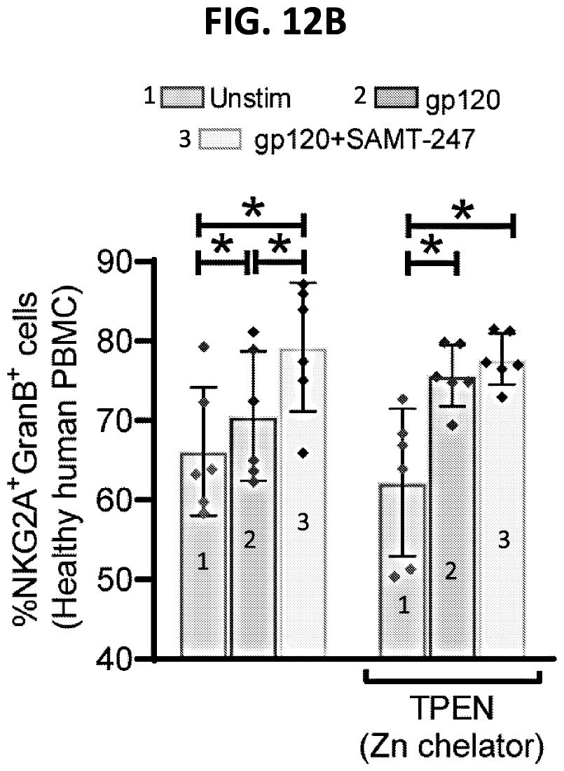

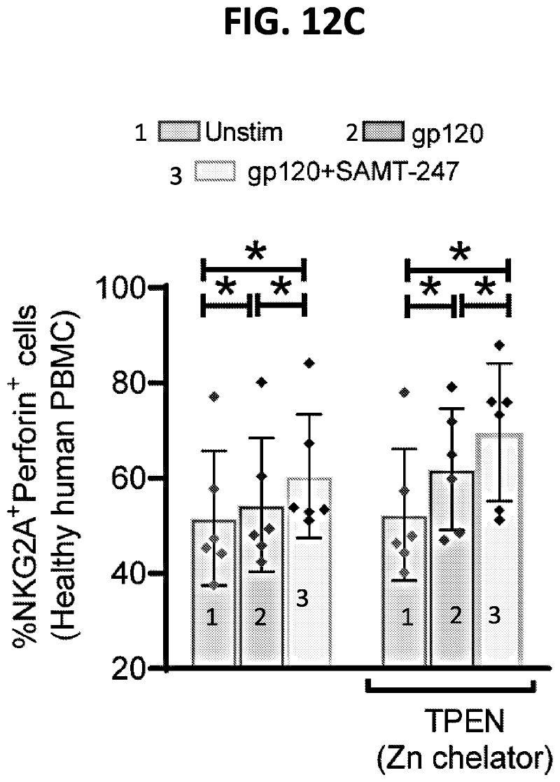

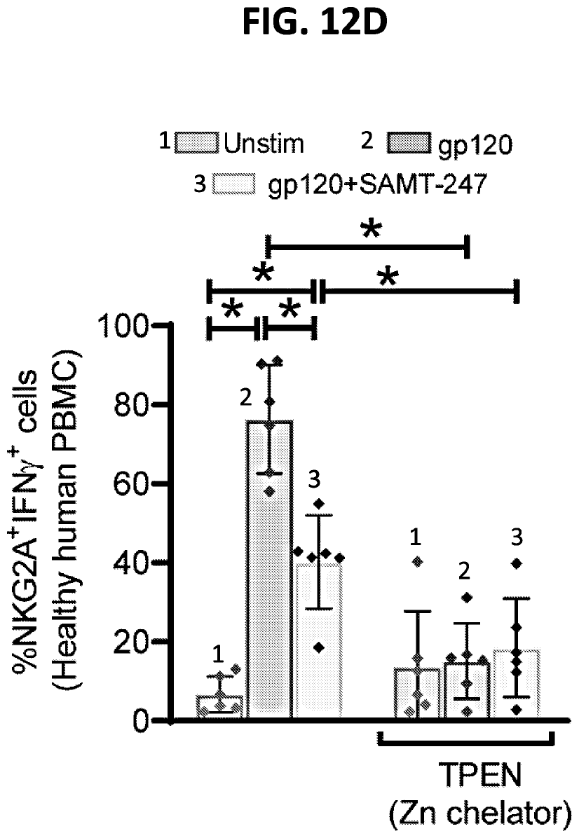

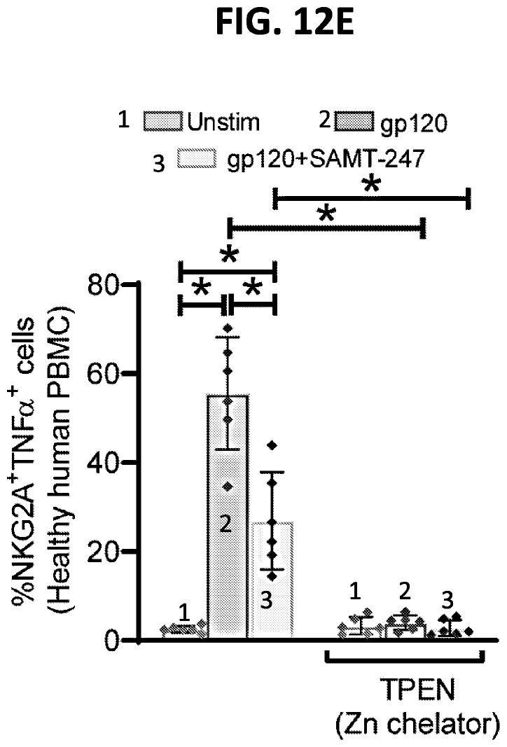

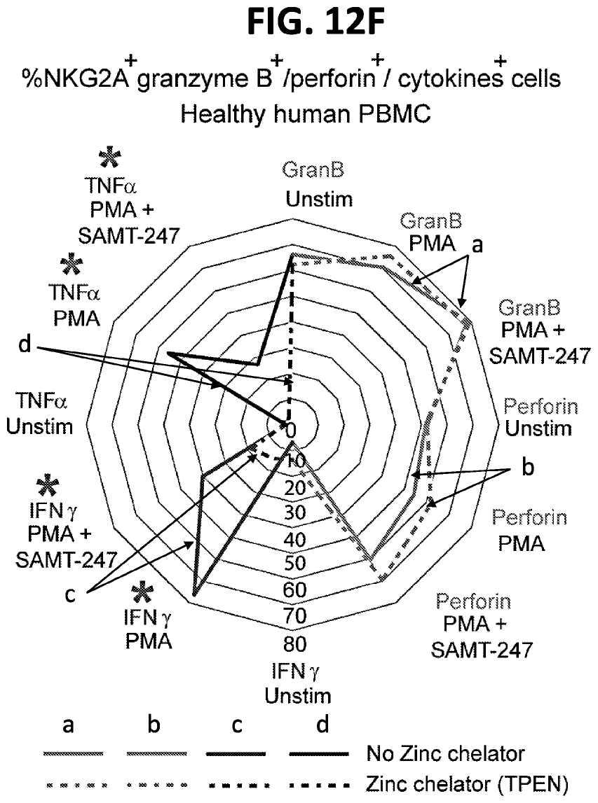

Next, the contribution of divalent free zinc to the immune responses correlating with protection was investigated using cryopreserved PBMCs obtained at the end of immunization (week 17) from vaccinated macaques. Macaque NK cells were stimulated with gp120 peptides in the presence or absence of SAMT-247 and of the membrane-permeable intracellular Zn chelator N, N,N′,N′ tetrakis-(2-pyridyl-methyl) ethylendiamine (TPEN). No significant changes were observed in SAMT-247's ability to increase granzyme B and perforin in NKG2A⁺ cells following gp120 peptide stimulation in the presence of TPEN, suggesting that this activity of SAMT-247 is not dependent on zinc (FIG. 6A and FIGS. 11A, 11B). In contrast, TPEN treatment significantly decreased both IFN-γ and TNF-α expression in gp120 peptide as well as gp120 peptide/SAMT-247 treated cells (FIG. 6B and FIGS. 11C, 11D). Interestingly, SAMT-247 treatment in the presence or absence of gp120 peptide increased the frequency of NKG2A⁺ cells, suggesting SAMT-247 increases the viability of NKG2A⁺ cells (FIG. 12A). As in macaque NKG2A⁺ cells, IFN-γ and TNF-α expression but not granzyme B or perforin were significantly reduced in human NKG2A⁺ cells following PMA stimulation with or without SAMT-247 treatment in the presence of TPEN (FIGS. 12B-12F). These results suggest that SAMT-247, by ejecting Zn, may affect the structural stability of transcription factors for cytokines, and that TPEN treatment further exacerbates this effect by sequestering intracellular free divalent Zn.

Several monocyte functions such as monocyte and macrophage phagocytosis are dependent on zinc, and can be restored by Zn supplements. Recent studies in humans also demonstrate that the level of intracellular zinc correlates with efferocytosis, in turn induced by pro-resolution IL-10 via an IL-10-mediated endocrine mechanism (Tone, K., et al., Kitasato Arch Exp Med 64, 263-269 (1991); Wirth, , et al., Immunology 68, 114-119 (1989); Hamon, R., et al. PLOS One 9, e1 10056 (2014)).

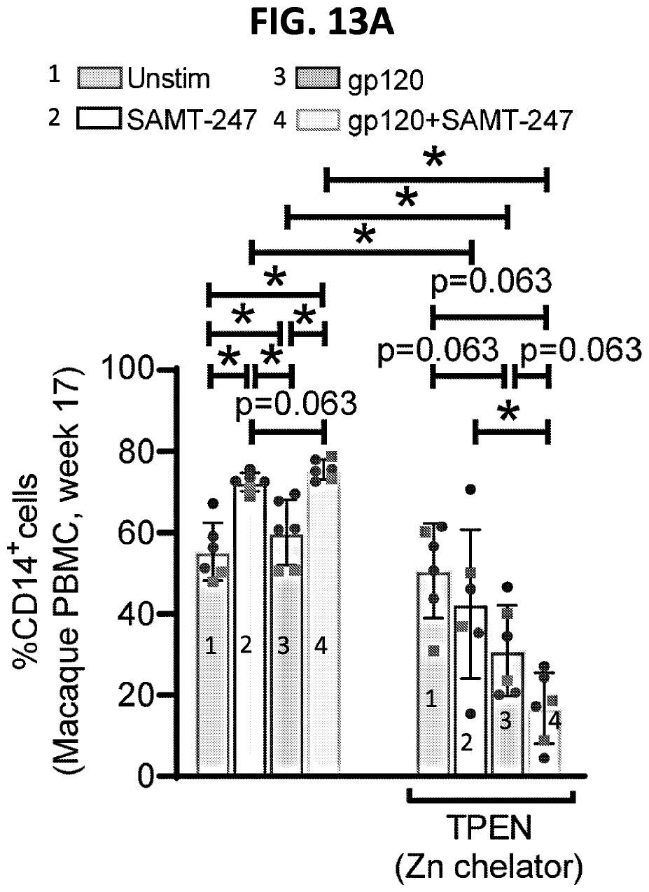

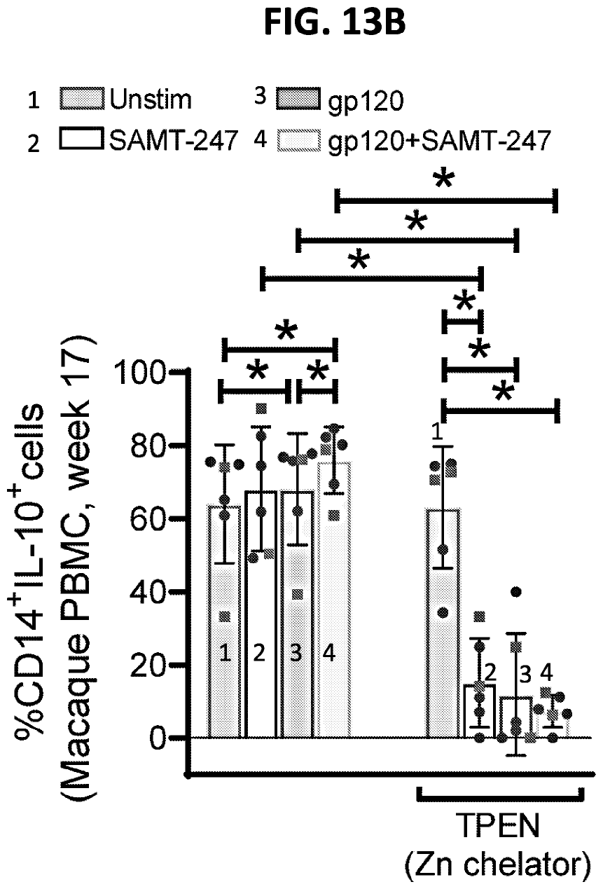

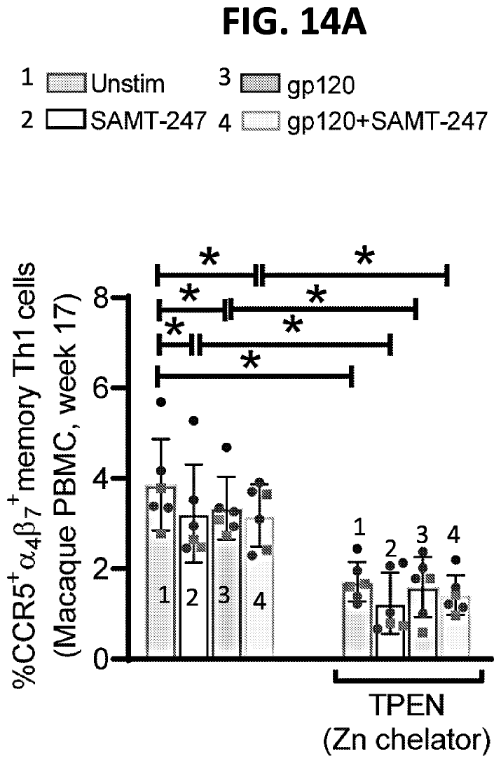

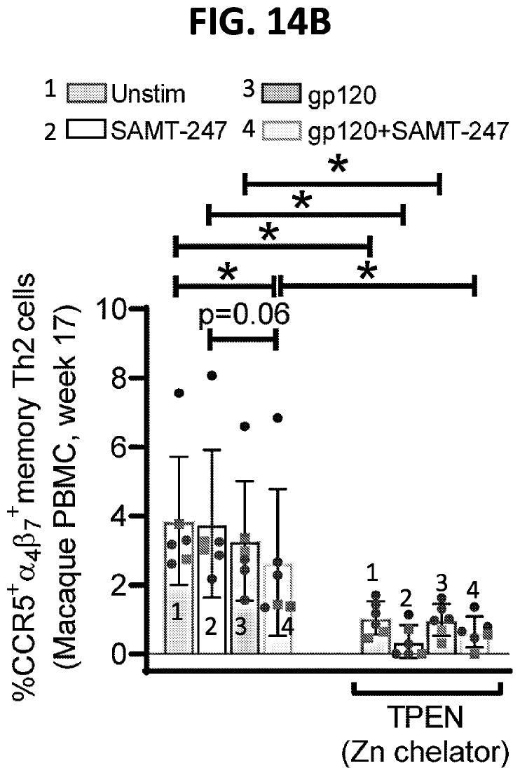

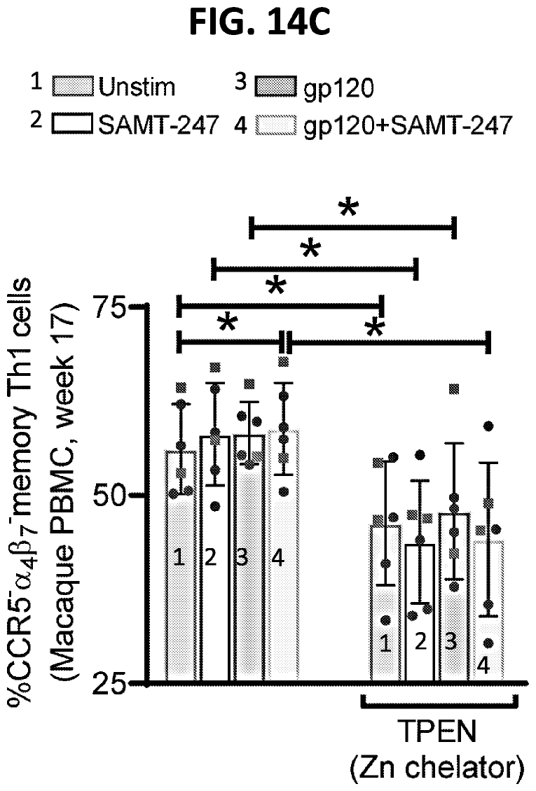

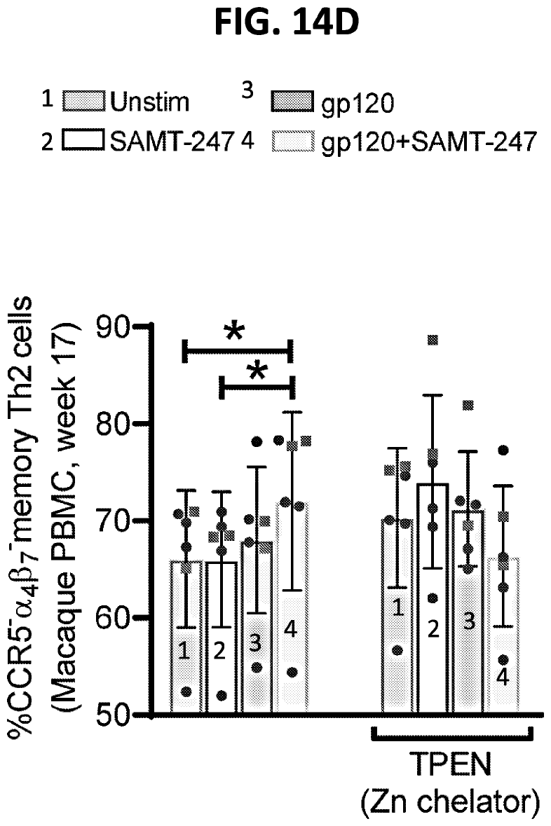

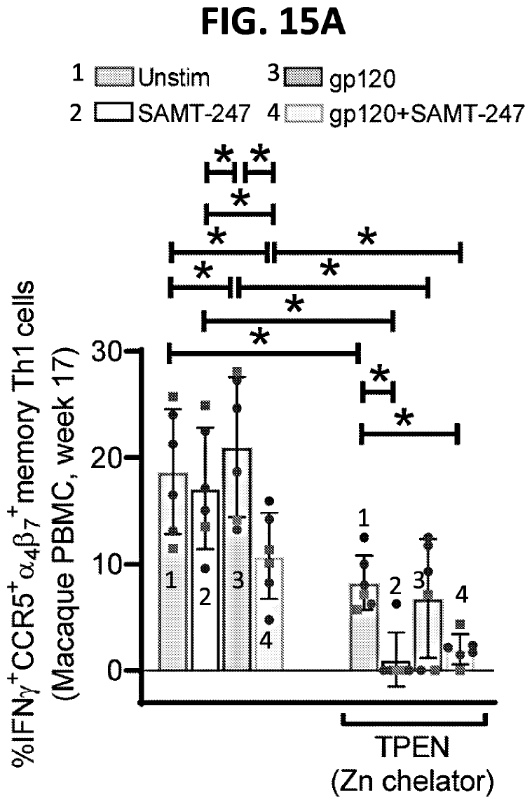

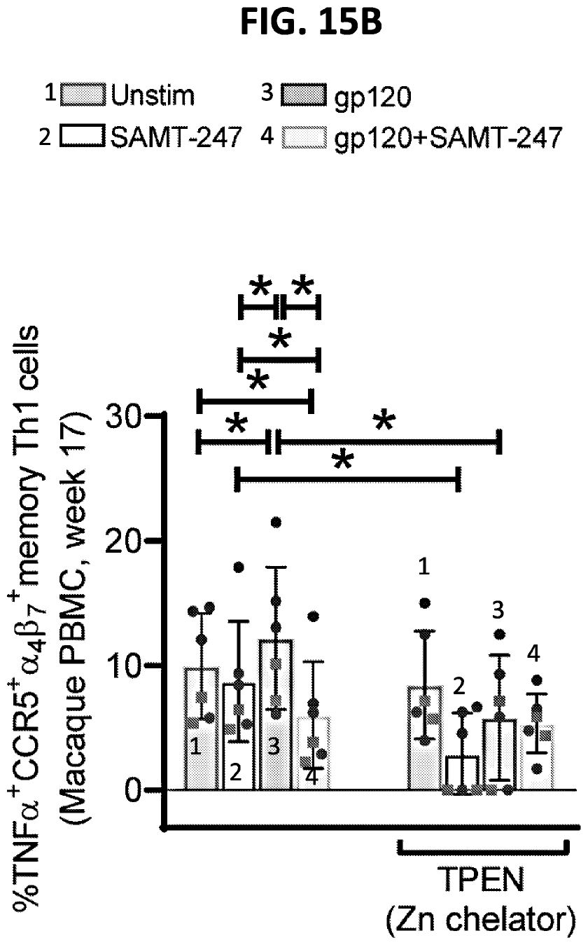

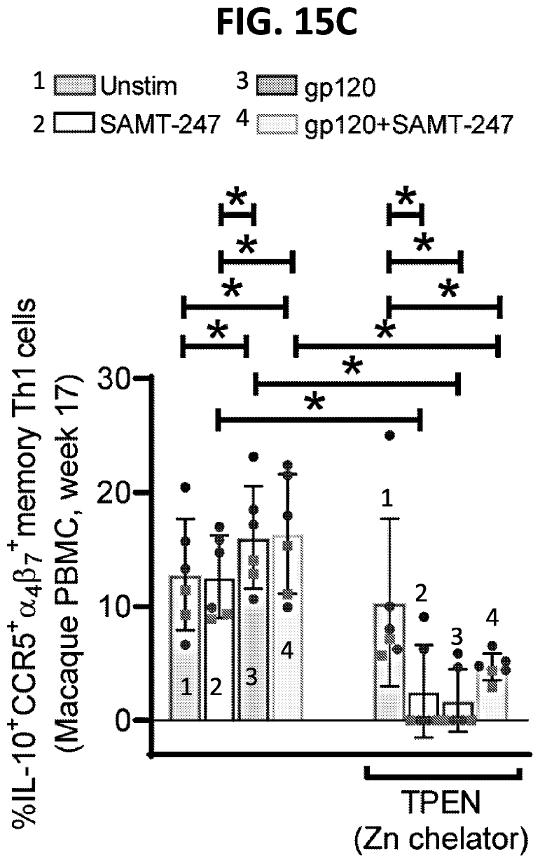

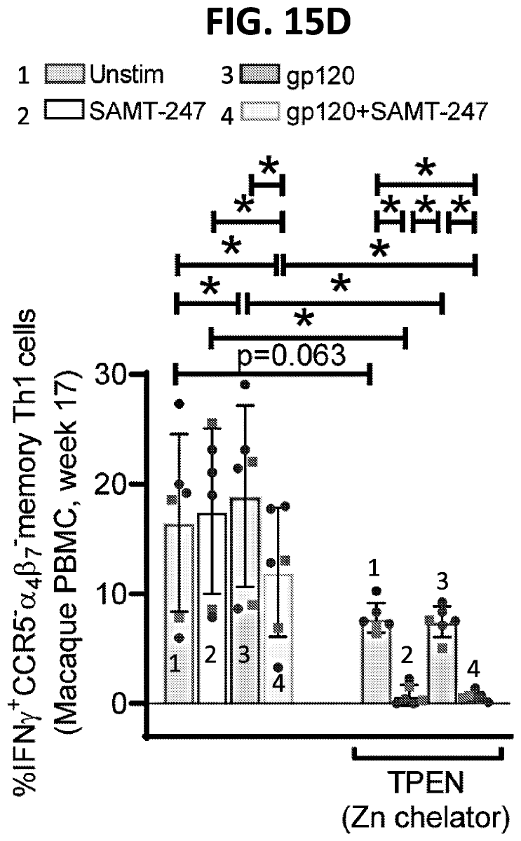

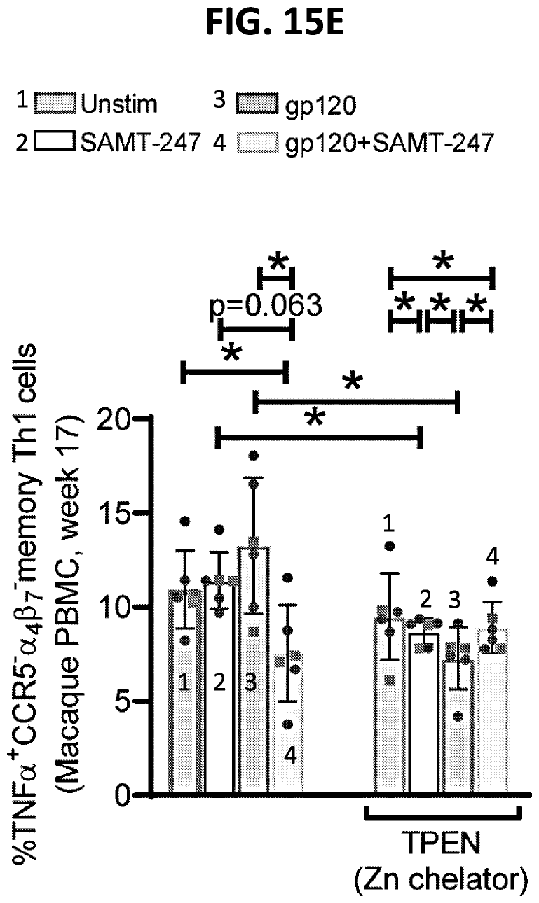

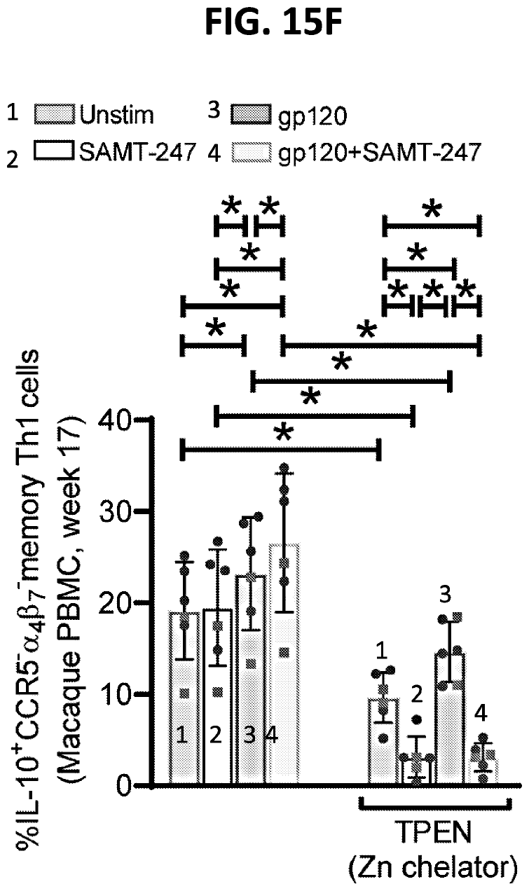

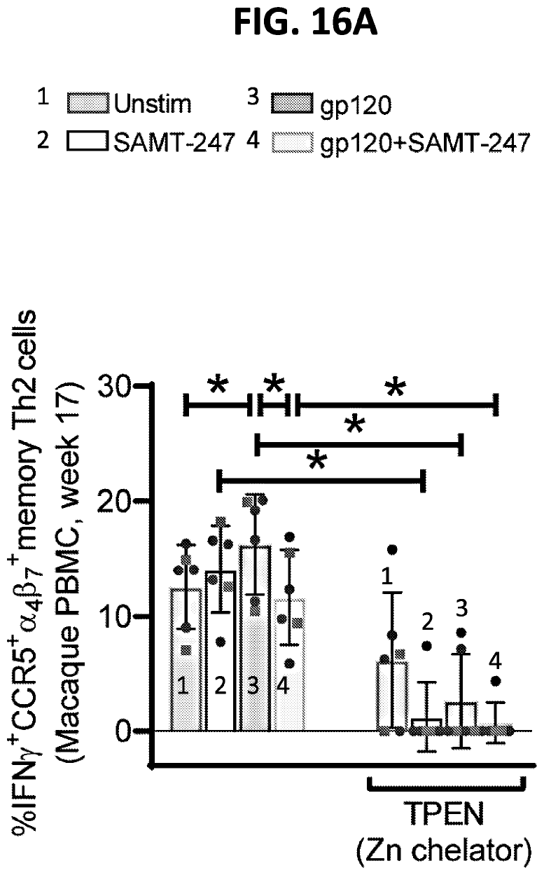

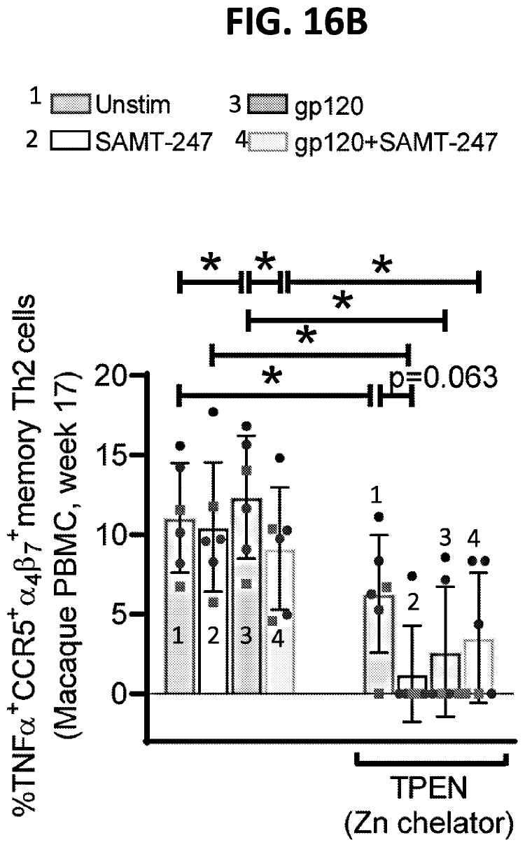

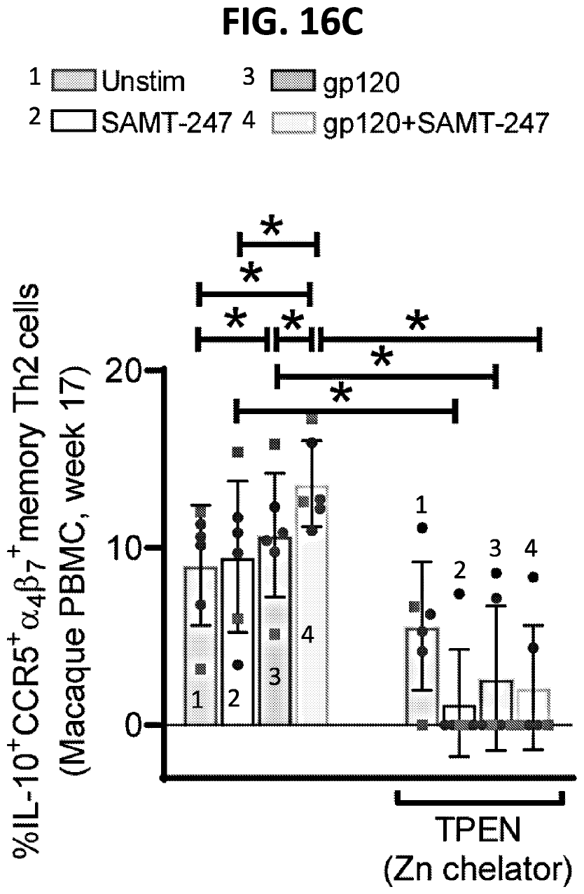

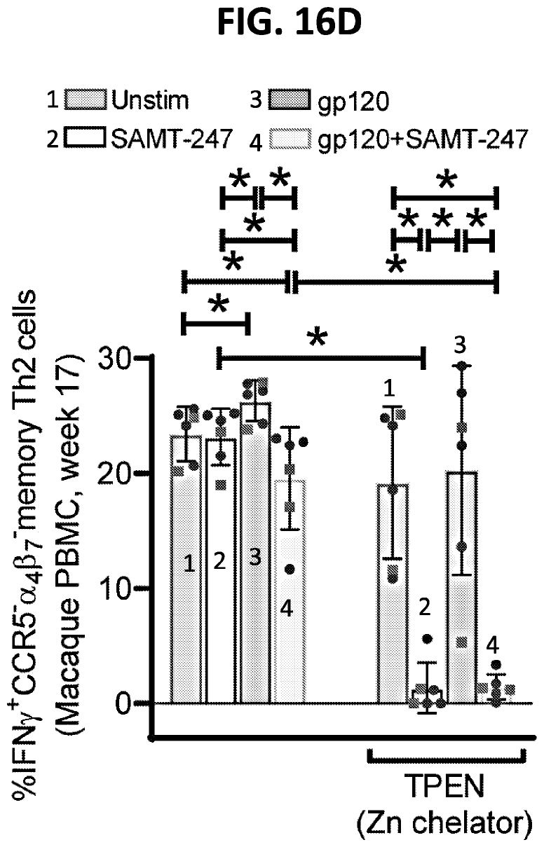

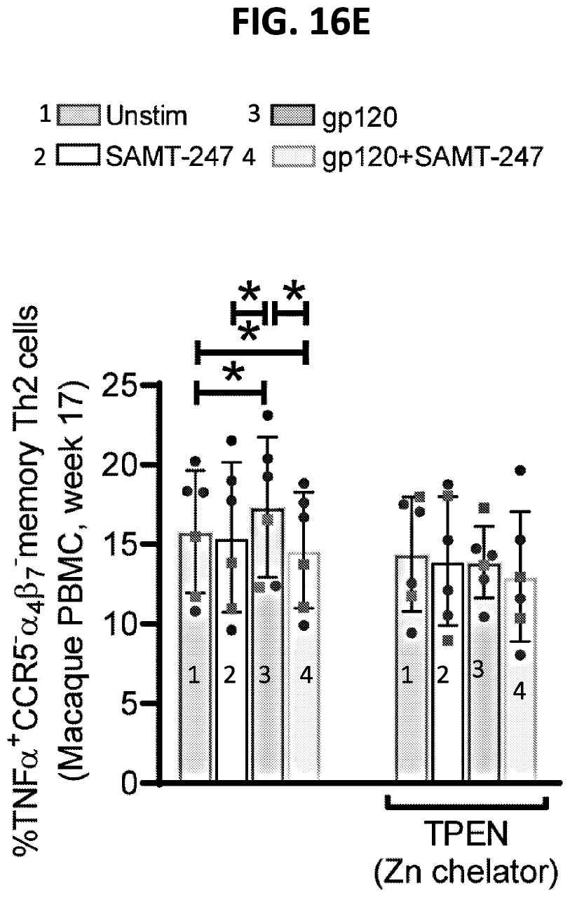

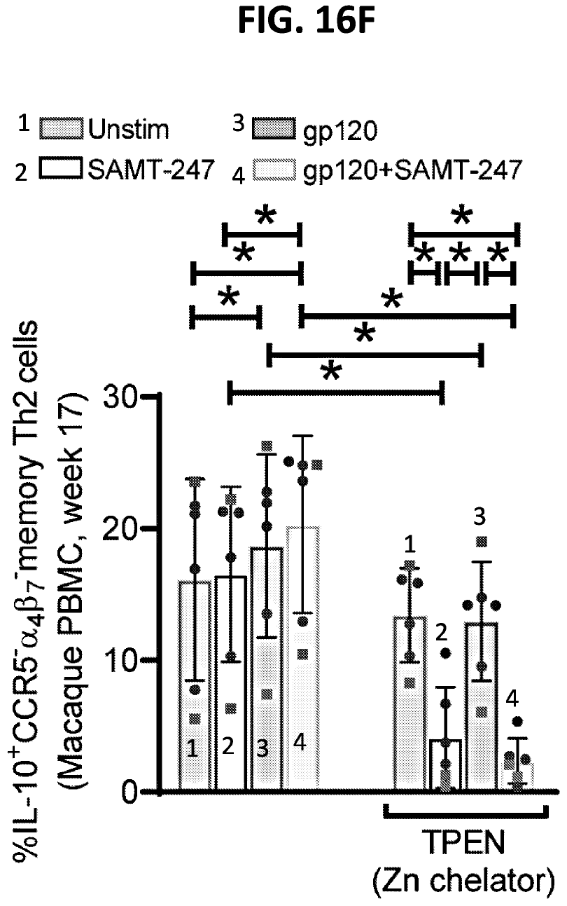

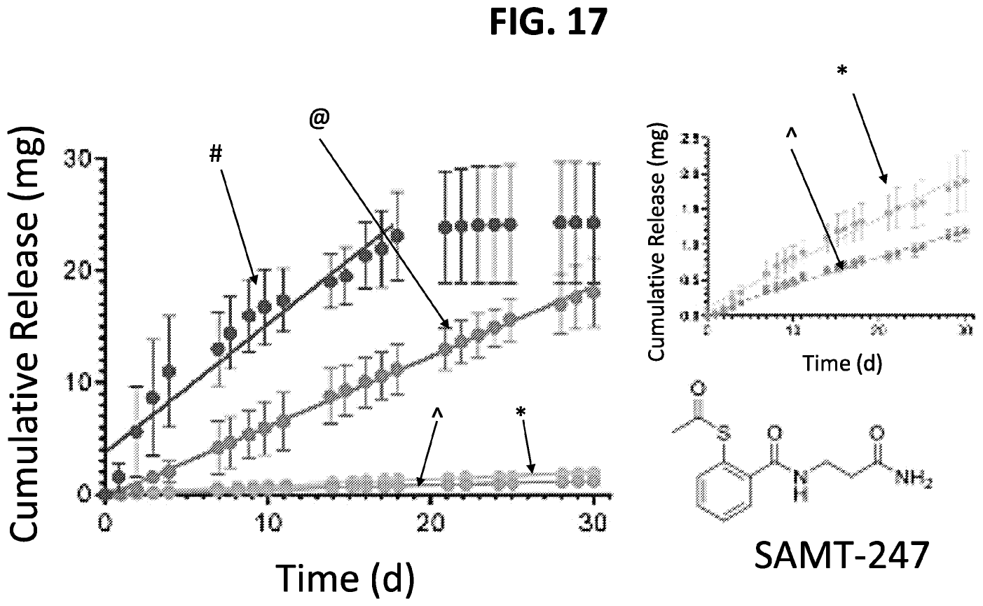

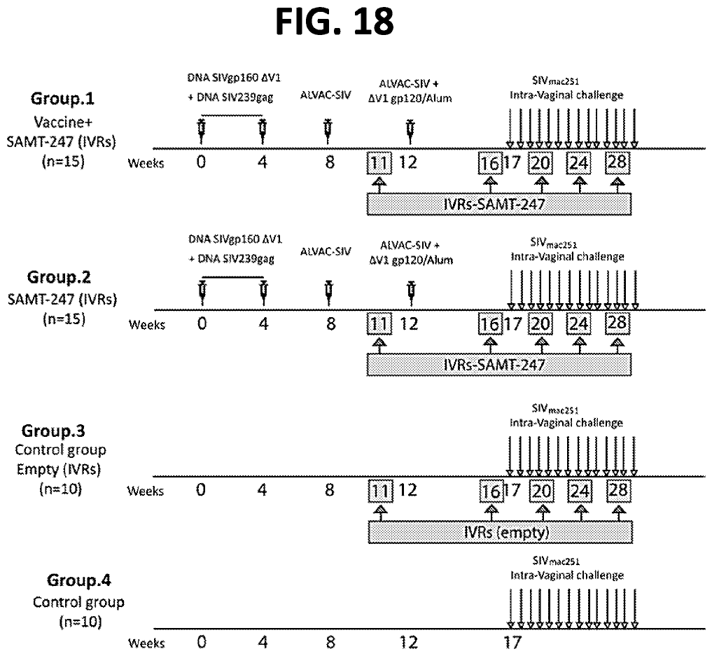

To assess the effect of chelation of free zinc on SAMT-247-associated CD14⁺ cell function, CD14⁺ cells were stimulated with gp120 pooled peptides in the presence or absence of SAMT-247 and TPEN. The SAMT-247 associated increase in CD14⁺ cells as well as the production of IL-10 were dependent on zinc, as demonstrated by the decreased frequency of CD14⁺ cells and IL-10 production by TPEN treatment (FIGS. 6C, 6D and FIGS. 13A-13B). These data are consistent with the notion that free Zn increases the survival and function of CD14⁺ cells.

Lastly, the effect of zinc was assessed on vaccine-induced (Ki67⁺) Th1 and Th2 CCR5^{+α}₄β₇⁺ or CCR5⁻α₄β₇⁻cells obtained at the end of immunization (week 17), following stimulation with gp120 pooled peptides, in the presence or absence of SAMT-247 and TPEN. Chelation of intracellular free zinc similarly affected the total percentage of both Th1 and Th2 cells (FIGS. 6E, 6F). In contrast, zinc chelation had a different outcome on CD4+ Th1 and Th2 subsets expressing (or not) CCR5. TPEN treatment resulted in a decreased percentage of both CCR5⁺ α₄β₇⁺Th1 and Th2 cells (FIGS. 14A, 14B), whereas Zn chelation decreased the number of Th1 CCR5⁻ α₄β₇⁻ cells, but not Th2 (FIGS. 14C, 14D). Analysis of cytokine production demonstrated that Zn chelation resulted in a decrease of IFN-γ and IL-10 in all conditions in both CCR5⁺α₄β₇⁺ Th1 and Th2 populations. TNF-α production was affected by zinc chelation in all conditions in CCR5⁺α₄β₇⁺ Th2 but only following stimulation with gp120 pooled peptides or SAMT-247 alone in CCR5⁺α₄β₇⁺ Th1 cells (FIG. 6G, 6H and FIGS. 15A-15F, FIGS. 16A-16F). In CCR5⁻α₄β₇⁻ Th1 cells, Zn chelation decreased IFN-γ, IL-10, and TNF-α in the same conditions as for CCR5⁺α₄β₇⁺ cells (FIG. 6I and FIGS. 16A-16F). In contrast, TPEN in Th2 CCR5⁻α₄β₇⁻ cells decreased INF-γ and IL-10, but not TNF-α production (FIG. 6J, FIGS. 16A-16F and Table 3).

TABLE 3
Effect of Zinc chelator on frequency of cellular marker expression

| | | | | | gp120 |
|---|---|---|---|---|---|
| | | | | gp120 | pooled |
| | | | SAMT- | pooled | peptide + |
| Cytokines | Cells | Unstim | 247 | peptide | SAMT-247 |
| GranB⁺ | NKG2A⁺ | — | — | — | — |
| Perforin⁺ | NKG2A⁺ | — | — | — | — |
| | CD14⁺ | — | ↓ | ↓ | ↓ |
| IFN-γ⁺ | NKG2A⁺ | — | — | ↓ | ↓ |
| | Th1⁺ | — | ↓ | — | ↓ |
| | Th1⁺CCR5⁺α₄β₇⁺ | ↓ | ↓ | ↓ | ↓ |
| | Th1⁺CCR5⁻α₄β₇⁻ | — | ↓ | ↓ | ↓ |
| | Th2⁺ | — | ↓ | ↓ | ↓ |
| | Th2⁺CCR5⁺α₄β₇⁺ | — | ↓ | ↓ | ↓ |
| | Th2⁺CCR5⁻α₄β₇⁻ | — | ↓ | — | ↓ |
| TNF-α⁺ | NKG2A⁺ | — | — | ↓ | ↓ |
| | Th1⁺ | — | ↓ | ↓ | — |
| | Th1⁺CCR5⁺α₄β₇⁺ | — | ↓ | ↓ | — |
| | Th1⁺CCR5⁻α₄β₇⁻ | — | ↓ | — | ↓ |
| | Th2⁺ | — | ↓ | — | ↓ |
| | Th2⁺CCR5⁺α₄β₇⁺ | ↓ | ↓ | ↓ | ↓ |
| | Th2⁺CCR5⁻α₄β₇⁻ | — | — | — | — |
| IL-10⁺ | CD14⁺ | — | ↓ | ↓ | ↓ |
| | Th1⁺ | — | ↓ | — | ↓ |
| | Th1⁺CCR5⁺α₄β₇⁺ | ↓ | ↓ | ↓ | ↓ |
| | Th1⁺CCR5⁻α₄β₇⁻ | ↓ | ↓ | ↓ | ↓ |
| | Th2⁺ | — | ↓ | — | ↓ |
| | Th2⁺CCR5⁺α₄β₇⁺ | ↓ | ↓ | ↓ | ↓ |
| | Th2⁺CCR5⁻α₄β₇⁻ | — | ↓ | ↓ | ↓ |

The macaque model has convincingly demonstrated the potential of the ALVAC/SIV vaccine modality boosted with gp120 (formulated in alum or MF59) (Vaccari, M., et al., Nat Med 22, 762-770 (2016)) by reproducing the efficacy of the successful RV144 HIV vaccine trial⁷ and accurately predicting the failure of the HVTN-702 trial in South Africa (Gray, G. E., et al., N Engl J Med 384, 1089-1100 (2021)). Prior work in the macaque model has moreover demonstrated that the efficacy of ALVAC-based HIV vaccine candidates can be improved by a DNA prime (Vaccari, M., et al. Nat Med 24, 847-856 (2018)), simplifying the vaccine regimen (Bissa et al., supra, 2022), and by exposing the Variable region 2 (V2) in an α-helix conformation via the deletion of V1 (Silva de Castro, I., et al., iScience 24, 102047 (2021)). While the main immune correlates of risk are well understood, the efficacy of the DNA/ALVAC/gp120/alum regimen remains suboptimal in both female and male macaques, reaching approximately a 70% decrease in the per/exposure risk of virus acquisition, and protecting only half of vaccinated animals from SIV_{mac251} acquisition (Bissa et al., supra, 2022).

To further decrease the risk of virus acquisition in vaccinated animals, it was investigated whether topical administration of the S-acyl-2 mercaptobenzamide thioester SAMT-247, which inhibits HIV maturation and infectivity (Jenkins, J. L. & Urban, L. Nat Chem Biol 6, 172-173 (2010)), would increase vaccine efficacy. Mechanistically, SAMT-247 causes unfolding and cross-linking of Gag polyprotein and ejects zinc, via selective acetylation of cysteine residues in the highly conserved zinc finger motif Cys-X₂-Cys-X₄-His-X₄-Cys of the HIV p7 nucleocapsid protein³⁸.

It was tested whether SAMT-247 could synergize with DNA/ALVAC/gp120/alum vaccination using a dose and regimen of the compound formulated in a gel preparation which, when given four hours before challenge exposure, was unable to decrease the risk of SIV_{mac251} acquisition (Helmold Hait, S., et al. J Immunol 204, 3315-3328 (2020)). Vaccination alone did decrease the risk of SIV_{mac251} acquisition by 65%, and SAMT-247 treatment alone had no effect. However, the combination of SAMT-247 gel with vaccination reduced the risk of vaginal SIV_{mac251} acquisition by 92.7% following 14 weekly vaginal exposures to SIV_{mac251} and protected 80% of female macaques from infection. The high level of synergy between vaccination and SAMT-247 suggested that the ability of SAMT-247 to eject zinc from transcription factors or enzymes may have affected local mucosal immunity and contributed to protection.

Zinc is an essential micronutrient and is a structural constituent for approximately 800 zinc-finger transcription factors (Lambert, S. A., et al., Cell 172, 650-665 (2018)) and 2000 enzymes (Andreini, C. & Bertini, I., J Inorg Biochem 111, 150-156 (2012)). Zinc transporters play a role in efferocytosis (Hamon, R., et al. PLOS One 9, e110056 (2014)) and intracellular zinc mobilization is triggered by the activation of the c-AMP pathway in a human pathogen (Kjellerup, L., et al., Front Microbiol 9, 502 (2018)). In addition, a dysfunction of Th2 responses linked to defective reprogramming of monocytes to anti-inflammatory M2 macrophages has been demonstrated in mice fed a zinc deficient diet (Kido, T., et al., H. Immunology 165, 445-459 (2022)). In humans, granulocyte and monocyte phagocytic function is augmented by zinc supplementation (Sheikh, A., et al. J Nutr 140, 1049-1056 (2010)).

In the presently disclosed study, it was observed that both ADCC and efferocytosis measured in blood, the main correlates of decreased risk afforded by the vaccine regimen, correlated with a decreased risk in vaccinated only animals, but not in those that received vaccine+SAMT-247. Thus, SAMT-247 may have augmented these protective responses at the mucosal site. Indeed, in vitro exposure to SAMT-247 of NK cells and monocytes obtained from vaccinated/SAMT-247 treated animals before challenge augmented both of these responses and restored both ADCC and efferocytosis correlations with decreased risk of virus acquisition following in vivo challenge. Based on these findings, NK, CD14⁺, Th1, and Th2 cells were stimulated in vitro with SAMT-247 alone or with PMA or SIV gp120 overlapping peptides. SAMT-247 following PMA stimulation increased granzyme and perforin in NKG2A⁺ cells, consistent with the observed increase in ADCC and IL17 production in mucosal NKp44 cells. Consistent with the observed increase of monocyte efferocytosis, SAMT-247 alone or together with gp120 peptides also increased IL-10 expression in CD14⁺ monocytes (Table 1). Chelation of zinc by TPEN demonstrated that the expression of IFN-γ and TNF-α, but not of granzyme and perforin in NK cells, and of IL-10 in monocytes is partly zinc-dependent. SAMT-247 in combination with gp120 peptides in T-cells increased the frequency of both Th1 and Th2 vaccine induced (Ki67⁺) CCR5^{-α}₄β₇⁻ cells, thereby decreasing target cells for the virus. In addition, the co-stimulation of SAMT-247 and gp120 peptides resulted in increased expression of IL-10 but decreased expression of both IFN-γ and TNF-α. All of these responses in T-cells were affected by chelation of zinc (Table 2). Therefore, it was concluded that SAMT-247 synergizes with the DNA/ALVAC/gp120/alum vaccine regimen by augmenting the immunological function of its effector cells. No effect was observed by SAMT-247 alone in naïve animals. It must be noted that the protective effect reported previously in the SIV_{mac251} model of SAMT-247 alone administered 3 hours prior to each challenge exposure was obtained in animals mock immunized with empty Ad vector and the alum adjuvant (Helmold Hait, S., et al. J Immunol 204, 3315-3328 (2020)), a key contributor to the immunological space created by the DNA/ALVAC/gp120/alum vaccine.

In summary, the data presented herein provide evidence that the combination of the DNA/ALVAC/gp120/alum vaccine and SAMT-247 is highly efficacious in preventing infection by a neutralization-resistant, highly pathogenic virus. The current data indicate that ADCC and efferocytosis are effectors of protection and point to a previously underestimated role of monocytes and NK cells in protection from HIV infection. Lastly, the data underscore the importance of pro-resolution anti-inflammatory responses able to maintain low levels of T-cell activation.

### Example 7

### Delivery of SAMT-247 from Intravaginal Rings

Intravaginal rings (IVRs) were developed that delivered the zinc finger inhibitor SAMT-247 over a wide range of in vitro release rates (FIG. 17). These data show that the rate of SAMT-247 delivery can be controlled over a wide range (0.039-1.15 mg d⁻¹) and with zero order kinetics. The pharmacokinetics (PKs) and preliminary safety of SAMT-247 IVRs have been evaluated in a rhesus macaque model in two 90-day studies. The devices were well-tolerated and did not lead to any local safety concerns.

### Example 8

### SIV_{mac251} Acquisition Rate in Macaques Treated with IVRs/SAM247, with or without Prior Vaccination

Twenty vaccinated female macaques and 18 unimmunized animals are vaginally inserted with IVRs/SAMT-247. Twelve animals will be kept naïve as control and receive IVRs without SAMT-247. All animals are exposed to 14 intravaginal challenges with SIV_{mac251} and the rate of virus acquisition is evaluated concurrently in the 12 naïve control animals. Data from 39 historical controls exposed by the same operators in the same animal facility to the same SIV_{mac251} dose are added to the data obtained from the concurrent 12 naïve controls in the present study.

The study is depicted in FIG. 18. Fifty juvenile cycling female macaques are used as follows: 20 macaques receive the vaccine-microbicide combination (group 1); 18 macaques receive the IVRs-SAMT-247 microbicide only (group 2); 12 naïve macaques receive empty IVRs as control (group 3).

Group sizes were determined based on previous vaccine studies comparing vaccinated macaques and controls. These historical control macaques and those administered microbicide were also included in the statistical analysis. Infection rates were assumed to equal those previously observed in the groups that received gel only or no treatment (0.333). Individual comparisons of the vaccine and combined groups against the control are predicted to have 82% and 99% power, respectively.

Study design: Macaques in group 1 are vaccinated intramuscularly at weeks 0 and 4 with DNA encoding SIVgp160_{ΔV1} (2 mg/macaque) and SIVgag (1 mg/macaque). At week 8, they receive ALVAC-SIV_{M766}gag-pro plus gp120-TM intramuscularly (10⁸ pfu in 1 ml PBS). At week 12, they again receive an intramuscular immunization with the ALVAC recombinant together with 400 μg of SIVgp120_{ΔV1} formulated in alum and administered in the opposite thigh. The macaques in group 3 remain naïve until challenge. The appropriate formulation of IRVs-SAMT-247 is used in the efficacy study.

IVRs-SAMT-247 are inserted in groups 1 and 2 one week before the final vaccination (week 11), and newly loaded rings are substituted at weeks 16, 20, 24 and 28. Animals in group 3 receive empty IVRs on the same schedule as groups 1 and 2 as control. Vaginal secretion obtained for three consecutive weeks from all animals to test for the concentration of SAMT-247 and SIV-specific antibodies. Five weeks following the final immunization of macaques in group 1, all macaques are weekly exposed intravaginally to SIV_{mac251} at a 1:25 dilution (800 TCID₅₀) as done in all historical controls and the risk of virus acquisition in treated and control animals assessed as done previously (Vaccari, M., et al., Nat Med, 2016. 22(7): p. 762-70; Silva de Castro, I., et al., Anti-V2 antibodies virus vulnerability revealed by envelope V1 deletion in HIV vaccine candidates. iScience, 2021(In Press); Vaccari, M., et al., Nat Med, 2018. 24(6): p. 847-856).

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method of inhibiting a human immunodeficiency virus (HIV) acquisition by a subject, comprising:
administering to the subject an effective amount of a recombinant gp120 protein comprising a deletion of HIV-1 Envelope (Env) residues 137-152 according to the HXBc2 numbering system, or a nucleic acid molecule encoding the recombinant gp120 protein, wherein the recombinant gp120 protein elicits an immune response to HIV-1; and
administering to the subject an effective amount of a SAMT-247 microbicide,
thereby inhibiting HIV acquisition by the subject.

2. The method of claim 1, wherein the recombinant gp120 protein comprises or consists of HIV-1 Env residues 31-507, with the deletion of residues 137-152, according to the HXBc2 numbering system.

3. The method of claim 1, wherein the recombinant gp120 protein comprises or consists of any one of SEQ ID NOs: 1-3.

4. The method of claim 1, comprising administering to the subject a first composition comprising the effective amount of the recombinant gp120 protein and a pharmaceutically acceptable carrier, and a second composition comprising the effective amount of the SAMT-247 microbicide.

5. The method of claim 4, wherein the first composition and the second composition are administered to the subject by different routes.

6. The method of claim 4, wherein the second composition is administered to the subject by a vaginal or rectal route.

7. The method of claim 6, wherein the second composition is a suppository, a cream or a gel.

8. The method of claim 6, wherein the second composition is administered by the vaginal route using a vaginal ring delivery device.

9. The method of claim 4, wherein the second composition is formulated for oral administration and is administered orally to the subject.

10. The method of claim 4, wherein the first composition further comprises an adjuvant.

11. The method of claim 10, wherein the adjuvant comprises an aluminum adjuvant.

12. The method of claim 10, wherein the adjuvant comprises monophosphoryl lipid A and/or saponin QS21.

13. The method of claim 1, comprising administering to the subject the effective amount of the nucleic acid molecule encoding the recombinant gp120.

14. The method of claim 13, comprising administering to the subject an effective amount of a vector encoding the recombinant gp120.

15. The method of claim 13, comprising administering to the subject an effective amount of an RNA encoding the recombinant gp120.

16. The method of claim 13, comprising administering to the subject an effective amount of a DNA encoding the recombinant gp120.

17. The method of claim 13, wherein the nucleic acid molecule encoding the recombinant gp120 encodes a gp160.

18. The method of claim 1, wherein the subject is a female human, and wherein the effective amount of the SAMT-247 microbicide is administered intravaginally.

19. The method of claim 1, wherein the effective amount of the SAMT-247 microbicide is administered within four hours of a potential exposure to HIV.

20. The method of claim 1, further comprising administering to the subject an effective amount of a booster vector encoding HIV-1 envelope (env) and polymerase (pol).

21. A method of inhibiting HIV-1 acquisition in a subject, comprising:
administering to the subject an effective amount of a composition comprising a prime immunization of a DNA vector encoding HIV-1 Env with a deletion of HIV-1 Env residues 137-152 according to the HXBc2 numbering system and an adjuvant,
administering to the subject a boost immunization of a vector encoding HIV env, HIV gag, and HIV pol and an alum adjuvant,
administering to the subject a boost immunization of a purified gp120 protein with a deletion of HIV-1 Env residues 137-152 according to the HXBc2 numbering system formulated with an effective amount of an alum adjuvant; and
applying intra-vaginally an effective amount of a SAMT-247 microbicide, thereby inhibiting the HIV-1 acquisition the subject.

22. The method of claim 21, wherein the SAMT-247 microbicide is administered within four hours of a potential HIV-1 exposure.

23. The method of claim 21, wherein the SAMT-247 microbicide is administered in an intravaginal ring prior to an HIV-1 exposure.


SEQUENCE LISTING (18 sequences)

SEQ ID NO: 1 - protein, 856 aa, Homo sapiens
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 476 aa, synthetic construct
NLWKWGTLILGLVIICSASDNLWVTVYYGVPVWRDADTTLFCASDAKAQETEAHNVWATHACVPTDPNPQELHLENVTENFNMWKNNMVEQMQEDVISLWDQSLKPCVKLTPLCVTLNCTNANLEVRNCSFNMTTELRDKKQKVHALFYKLDIVPIEDNTSSSKYRLINCNTSVIKQACPKISFDPIPIHYCTPAGYAILKCNDKNFNGTGPCKNVSSVQCTHGIKPAVSTQLLLNGSLAEEEIIIRSENLTNNAKTIIVHLNKSVEINCTRPSNNTRTSINIGPGQVFYRTGDIIGDIRKAYCEINGAKWNEVLKKVTEKLKEHFNNKTIIFQPPSGGDLEITMHHFNCRGEFFYCNTTRLFNNTCMENETMEGCNGTIILPCKIKQIINMWQRAGQAMYAPPISGRINCVSNITGILLTRDGGLNNTNETFRPGGGNIKDNWRSELYKYKVVQIEPLGIAPTRAKRRVVEREKR

SEQ ID NO: 3 - protein, 480 aa, synthetic construct
HWWGWGTMLLGLLMICSATEKLWVTVYYGVPVWKEATTTLFCASDAKAYDTEVHNVWATHACVPTDPNPQEVQLVNVTEDFNMWKNNMVEQMHEDIISLWDQSLKPCVKLTPLCVTLNCTDLRNEMKNCSFNITTSIRDKMQKEYALLYKLDIVAIDKDNTSYRLISCNTSVITQACPKVSFEPIPIHYCAPAGFAILKCNDKNFTGKGPCKNVSTVQCTHGIRPVVSTQLLLNGSLAEEEVVIRSENFTNNAKTIIVHLNESVQINCTRPYNNRRTRIHIGPGRAFYTTKNIKGTIRQAHCTISSAKWNDTLRQIVSKLKEQFKNKTIVFKQSSGGDPEIVMHSFNCGGEFFYCNTSSLFNSTWNGNNTWNNTTGSNSNITLQCKIKQIINMWQEVGKAMYAPPIEGQIRCSSNITGLLLTRDGGNDTDTNNTEIFRPGGGDMRDNWRSELYKYKVVTIEPLGVAPTKAKRRVVQREKR

SEQ ID NO: 4 - protein, 484 aa, synthetic construct
RWWTWGILGFWMLMICNVWGNLWVTVYYGVPVWKEAKTTLFCASDAKSYEKEVHNVWATHACVPTDPNPQEIVLGNVTENFNMWKNDMVDQMHEDIISLWDQSLKPCVKLTPLCVTLNCTEVNVDMKNCSFNITTELKDKKKNVYALFYKLDIVSLNETDDSETGNSSKYYRLINCNTSALTQACPKVSFDPIPIHYCAPAGYAILKCNNKTFNGTGPCHNVSTVQCTHGIKPVVSTQLLLNGSLAEEGIIIRSENLTNNVKTIIVHLNRSIEIVCVRPNNNTRQSIRIGPGQTFYATGDIIGDIRQAHCNISRTNWTKTLREVRNKLREHFPNKNITFKPSSGGDLEITTHSFNCRGEFFYCNTSGLFSINYTENNTDGTPITLPCRIRQIINMWQEVGRAMYAPPIEGNIACKSDITGLLLVRDGGSTNDSTNNNTEIFRPAGGDMRDNWRSELYKYKVVEIKPLGIAPTEAKRRVVEREKR

SEQ ID NO: 5 - protein, 828 aa, synthetic construct
NLWKWGTLILGLVIICSASDNLWVTVYYGVPVWRDADTTLFCASDAKAQETEAHNVWATHACVPTDPNPQELHLENVTENFNMWKNNMVEQMQEDVISLWDQSLKPCVKLTPLCVTLNCTNANLEVRNCSFNMTTELRDKKQKVHALFYKLDIVPIEDNTSSSKYRLINCNTSVIKQACPKISFDPIPIHYCTPAGYAILKCNDKNFNGTGPCKNVSSVQCTHGIKPAVSTQLLLNGSLAEEEIIIRSENLTNNAKTIIVHLNKSVEINCTRPSNNTRTSINIGPGQVFYRTGDIIGDIRKAYCEINGAKWNEVLKKVTEKLKEHFNNKTIIFQPPSGGDLEITMHHFNCRGEFFYCNTTRLFNNTCMENETMEGCNGTIILPCKIKQIINMWQRAGQAMYAPPISGRINCVSNITGILLTRDGGLNNTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWNSTWSNRSFEEIWNNMTWIEWEREISNYTNQIYEILTQSQNQQDRNEKDLLELDKWASLWKWFDITNWLWYIKIFIMIVGGLIGLRIIFAVLSIVNRVRQGYSPLSLQIPTHHQREPDRPERIEEGGGEQGRDKSVRLVSGFLALTWDDLRSLCLFSYHRLRDFISIAARTVELLGHSSLKGLRRGWEGLKYLGNLILYWGQELKISAISLLNATAIAVAGWTDRVIEVAQGAWRAILHIPRRIRQGLERTLL

SEQ ID NO: 6 - protein, 824 aa, synthetic construct
HWWGWGTMLLGLLMICSATEKLWVTVYYGVPVWKEATTTLFCASDAKAYDTEVHNVWATHACVPTDPNPQEVQLVNVTEDFNMWKNNMVEQMHEDIISLWDQSLKPCVKLTPLCVTLNCTDLRNEMKNCSFNITTSIRDKMQKEYALLYKLDIVAIDKDNTSYRLISCNTSVITQACPKVSFEPIPIHYCAPAGFAILKCNDKNFTGKGPCKNVSTVQCTHGIRPVVSTQLLLNGSLAEEEVVIRSENFTNNAKTIIVHLNESVQINCTRPYNNRRTRIHIGPGRAFYTTKNIKGTIRQAHCTISSAKWNDTLRQIVSKLKEQFKNKTIVFKQSSGGDPEIVMHSFNCGGEFFYCNTSSLFNSTWNGNNTWNNTTGSNSNITLQCKIKQIINMWQEVGKAMYAPPIEGQIRCSSNITGLLLTRDGGNDTDTNNTEIFRPGGGDMRDNWRSELYKYKVVTIEPLGVAPTKAKRRVVQREKRAAIGALFLGFLGAAGSTMGAASMMLTVQARQLLSGIVQQQNNLLRAIEAQQHMLQLTVWGIKQLQARVLAVERYLRDQQLLGIWGCSGKLICTTTVPWNASWSNKSQEDIWNNMTWMQWEREIDNYTSTIYELLEKSQNQQEKNEQELLELDKWASLWNWFDITNWLWYIKIFIMIVGGLIGLRIVFAVLSIVNRVRQGYSPLSLQTRPPVPRGPDRPEGTEEEGGERDRDTSGRLVDGFLAIIWVDLRSLLLFSYHRLRDLLLIAARIVELLGRRGWEILKYWWNLLQYWSQELKNSAVSLLNATAVAVAEGTDRVIEVLQRAGRAILHIPTRIRQGLERALL

SEQ ID NO: 7 - protein, 836 aa, synthetic construct
RWWTWGILGFWMLMICNVWGNLWVTVYYGVPVWKEAKTTLFCASDAKSYEKEVHNVWATHACVPTDPNPQEIVLGNVTENFNMWKNDMVDQMHEDIISLWDQSLKPCVKLTPLCVTLNCTEVNVDMKNCSFNITTELKDKKKNVYALFYKLDIVSLNETDDSETGNSSKYYRLINCNTSALTQACPKVSFDPIPIHYCAPAGYAILKCNNKTFNGTGPCHNVSTVQCTHGIKPVVSTQLLLNGSLAEEGIIIRSENLTNNVKTIIVHLNRSIEIVCVRPNNNTRQSIRIGPGQTFYATGDIIGDIRQAHCNISRTNWTKTLREVRNKLREHFPNKNITFKPSSGGDLEITTHSFNCRGEFFYCNTSGLFSINYTENNTDGTPITLPCRIRQIINMWQEVGRAMYAPPIEGNIACKSDITGLLLVRDGGSTNDSTNNNTEIFRPAGGDMRDNWRSELYKYKVVEIKPLGIAPTEAKRRVVEREKRAVGIGAVFLGFLGAAGSTMGAASITLTAQARQVLSGIVQQQSNLLRAIEAQQHLLQLTVWGIKQLQTRVLAIERYLKDQQLLGLWGCSGKLICTTAVPWNISWSNKSKTDIWDNMTWMQWDREISNYTNTIYRLLEDSQSQQEQNEKDLLALDSWNNLWNWFDITKWLWYIKIFIMIVGGLIGLRIIFAVLSIVNRVRQGYSPLSFQTLIPNPREPDRPGRIEEEGGEQDKERSVRLVSGFLALAWDDLRSLCLFSYHRLRDFILVTARAVELLRRSSLKGLQRGWEALKYLGSLVQYWGLELKKSAISLLDTIAIAVAEGTDRIIELIQGICRAIRNVPRRIRQGFETALL

SEQ ID NO: 8 - protein, 839 aa, synthetic construct
MRVKETQMTWPNLWKWGTLILGLVIICSASDNLWVTVYYGVPVWRDADTTLFCASDAKAQETEAHNVWATHACVPTDPNPQELHLENVTENFNMWKNNMVEQMQEDVISLWDQSLKPCVKLTPLCVTLNCTNANLEVRNCSFNMTTELRDKKQKVHALFYKLDIVPIEDNTSSSKYRLINCNTSVIKQACPKISFDPIPIHYCTPAGYAILKCNDKNFNGTGPCKNVSSVQCTHGIKPAVSTQLLLNGSLAEEEIIIRSENLTNNAKTIIVHLNKSVEINCTRPSNNTRTSINIGPGQVFYRTGDIIGDIRKAYCEINGAKWNEVLKKVTEKLKEHFNNKTIIFQPPSGGDLEITMHHFNCRGEFFYCNTTRLFNNTCMENETMEGCNGTIILPCKIKQIINMWQRAGQAMYAPPISGRINCVSNITGILLTRDGGLNNTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWNSTWSNRSFEEIWNNMTWIEWEREISNYTNQIYEILTQSQNQQDRNEKDLLELDKWASLWKWFDITNWLWYIKIFIMIVGGLIGLRIIFAVLSIVNRVRQGYSPLSLQIPTHHQREPDRPERIEEGGGEQGRDKSVRLVSGFLALTWDDLRSLCLFSYHRLRDFISIAARTVELLGHSSLKGLRRGWEGLKYLGNLILYWGQELKISAISLLNATAIAVAGWTDRVIEVAQGAWRAILHIPRRIRQGLERTLL

SEQ ID NO: 9 - protein, 835 aa, synthetic construct
MRVKGIRRNYQHWWGWGTMLLGLLMICSATEKLWVTVYYGVPVWKEATTTLFCASDAKAYDTEVHNVWATHACVPTDPNPQEVQLVNVTEDFNMWKNNMVEQMHEDIISLWDQSLKPCVKLTPLCVTLNCTDLRNEMKNCSFNITTSIRDKMQKEYALLYKLDIVAIDKDNTSYRLISCNTSVITQACPKVSFEPIPIHYCAPAGFAILKCNDKNFTGKGPCKNVSTVQCTHGIRPVVSTQLLLNGSLAEEEVVIRSENFTNNAKTIIVHLNESVQINCTRPYNNRRTRIHIGPGRAFYTTKNIKGTIRQAHCTISSAKWNDTLRQIVSKLKEQFKNKTIVFKQSSGGDPEIVMHSFNCGGEFFYCNTSSLFNSTWNGNNTWNNTTGSNSNITLQCKIKQIINMWQEVGKAMYAPPIEGQIRCSSNITGLLLTRDGGNDTDTNNTEIFRPGGGDMRDNWRSELYKYKVVTIEPLGVAPTKAKRRVVQREKRAAIGALFLGFLGAAGSTMGAASMMLTVQARQLLSGIVQQQNNLLRAIEAQQHMLQLTVWGIKQLQARVLAVERYLRDQQLLGIWGCSGKLICTTTVPWNASWSNKSQEDIWNNMTWMQWEREIDNYTSTIYELLEKSQNQQEKNEQELLELDKWASLWNWFDITNWLWYIKIFIMIVGGLIGLRIVFAVLSIVNRVRQGYSPLSLQTRPPVPRGPDRPEGTEEEGGERDRDTSGRLVDGFLAIIWVDLRSLLLFSYHRLRDLLLIAARIVELLGRRGWEILKYWWNLLQYWSQELKNSAVSLLNATAVAVAEGTDRVIEVLQRAGRAILHIPTRIRQGLERALL

SEQ ID NO: 10 - protein, 847 aa, synthetic construct
MRVREILRNWQRWWTWGILGFWMLMICNVWGNLWVTVYYGVPVWKEAKTTLFCASDAKSYEKEVHNVWATHACVPTDPNPQEIVLGNVTENFNMWKNDMVDQMHEDIISLWDQSLKPCVKLTPLCVTLNCTEVNVDMKNCSFNITTELKDKKKNVYALFYKLDIVSLNETDDSETGNSSKYYRLINCNTSALTQACPKVSFDPIPIHYCAPAGYAILKCNNKTFNGTGPCHNVSTVQCTHGIKPVVSTQLLLNGSLAEEGIIIRSENLTNNVKTIIVHLNRSIEIVCVRPNNNTRQSIRIGPGQTFYATGDIIGDIRQAHCNISRTNWTKTLREVRNKLREHFPNKNITFKPSSGGDLEITTHSFNCRGEFFYCNTSGLFSINYTENNTDGTPITLPCRIRQIINMWQEVGRAMYAPPIEGNIACKSDITGLLLVRDGGSTNDSTNNNTEIFRPAGGDMRDNWRSELYKYKVVEIKPLGIAPTEAKRRVVEREKRAVGIGAVFLGFLGAAGSTMGAASITLTAQARQVLSGIVQQQSNLLRAIEAQQHLLQLTVWGIKQLQTRVLAIERYLKDQQLLGLWGCSGKLICTTAVPWNISWSNKSKTDIWDNMTWMQWDREISNYTNTIYRLLEDSQSQQEQNEKDLLALDSWNNLWNWFDITKWLWYIKIFIMIVGGLIGLRIIFAVLSIVNRVRQGYSPLSFQTLIPNPREPDRPGRIEEEGGEQDKERSVRLVSGFLALAWDDLRSLCLFSYHRLRDFILVTARAVELLRRSSLKGLQRGWEALKYLGSLVQYWGLELKKSAISLLDTIAIAVAEGTDRIIELIQGICRAIRNVPRRIRQGFETALL

SEQ ID NO: 11 - protein, 4 aa, synthetic construct
REKR

SEQ ID NO: 12 - protein, 6 aa, synthetic construct
RRRRRR

SEQ ID NO: 13 - protein, 10 aa, synthetic construct
GGSGGGGSGG

SEQ ID NO: 14 - protein, 27 aa, synthetic construct
KIFIMIVGGLIGLRIVFAVLSVIHRVR

SEQ ID NO: 15 - protein, 23 aa, Influenza A virus
ILAIYSTVASSLVLLVSLGAISF

SEQ ID NO: 16 - protein, 29 aa, Influenza A virus
IITIGSICMVVGIISLILQIGNIISIWVS

SEQ ID NO: 17 - protein, 26 aa, synthetic construct
GYIPEAPRDGQAYVRKDGEWVLLSTF

SEQ ID NO: 18 - DNA, 2526 bp, synthetic construct
  source 1..2526: mol_type = other DNA
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacagaagtgcacaatgtctgggccacacatgcctgtgtacccacagaccccaacccacaagaaatagacctggaaaatgtaacagaaaattttaacatgtggaaaaataacatggtagagcagatgcaggaggatgtaatcagtttatgggatcaaagtctaaagccatgtgtaaagttaactcctctctgcgttactttacattgtactaatgctaatttggaagtaagaaactgttcttttaatatgaccacagaactaagagataagaagcagaaggtccatgcacttttttataagcttgatatagtaccaattgaagataataacgataatagtaagtataggttaataaattgtaatacttcagtcattaagcaggcttgtccaaagatatcctttgatccaattcctatacattattgtactccagctggttatgcgattttaaagtgtaatgataagaatttcaatgggacagggccatgtaaaaacgtcagctcagtacaatgcacacatggaattaagccagtggtatcaactcaattgctgttaaatggcagtctagcagaagaagagataataatcagatctgaagatctcacaaacaatgccaaaaccataatagtgcaccttaataaatctgtagtaatcaattgtaccagaccctccaacaatacaagaacaagtataactataggaccaggacaagtattctatagaacaggagacataataggagatataagaaaagcatattgtgagattaatggaacagaatggaataaagctttaaaacaggtaactgaaaagttaaaagagcactttaataataagccaataatctttcaaccaccctcaggaggagatctagaaattacaatgcatcattttaattgtagaggagaatttttctattgcaatacaacacgactgtttaataatacttgcatagcaaatggaaccatagaggggtgtaatggcaatatcacacttccatgcaagataaaacaaattataaacatgtggcagggagcaggacaagcaatgtatgctcctcccatcagtggaacaattaattgtgtatcaaatattacaggaatactattgacaagagatggtggtgctactaataatacgaataacgagaccttcagacctggaggaggaaatataaaggacaattggagaaatgaattatataaatataaagtagtacaaattgaaccactaggagcagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaaagcaatttgctgagggctatagaggcgcagcagcatctgttgcaactcacagtctggggcattaaacagctccaggcaagagtcctggctgtggaaagatacctaaaggatcaaaagttcctaggactttggggctgctctggaaaaatcatctgcaccactgcagtgccctggaactccacttggagtaataaatctcttgaagagatttggaacaacatgacatggatagaatgggagagagaaattagcaattacacaaaccaaatatatgagatacttacaaaatcgcaggaccagcaggacaggaatgaaaaggatttgttagaattggataaatgggcaagtctgtggacttggtttgacataacaaattggctgtggtatataaaaatatttataatgatagtgggaggtttaataggattaagaataatttttgctgtgctttctatagtgaatagagttaggcagggatactcacctttgtctttccagaccccttgccatcatcagagggaacccgacagacccgaaagaatcgaagaagaaggtggcgagcaaggcagagacagatccgtgcgattagtgagcggattcttagctcttgcatgggacgatctacggagcctgtgcctcttcagctaccaccgcttgagagacttcatcttgattgcagcgaggactgtggaacttctgggacgcagcagtctcaagggactgagacgggggtgggaaggcctcaaatatctggggaatcttctgttatattggggtcaggaactaaaaattagtgctatttctttgcttgatgctacagcaatagcagtagcggggtggacagatagggttatagaagtagcacaaggagcttggaaagccattctccacatacctagaagaatcagacagggcttagaaagggctttgcaataa